(19) United States
(12) Patent Application Publication
(10) Pub. No.: US 2024/0316318 A1
(43) Pub. Date: Sep. 26, 2024

(54) STEERABLE CATHETER

(71) Applicant: Edwards Lifesciences Innovation (Israel) Ltd., Caesarea (IL)

(72) Inventors: Eyal Shaolian, Newport Beach, CA (US); Mauricio Guerrero, Hawthorne, CA (US); Khalid Jamal, Irvine, CA (US)

(21) Appl. No.: 18/737,750

(22) Filed: Jun. 7, 2024

Related U.S. Application Data
(63) Continuation of application No. PCT/IB2022/061743, filed on Dec. 3, 2022.
(60) Provisional application No. 63/288,506, filed on Dec. 10, 2021.

(51) Int. Cl. A61M 25/01; A61M 25/00

(52) U.S. Cl. CPC A61M 25/0136; A61M 25/0097

(57) ABSTRACT

A catheter assembly includes a catheter and at least one actuation element. The catheter has a shaft extending from a proximal end to a distal end. In some implementations, the catheter has a telescopic articulation device at a distal end of the catheter. The telescopic articulation device is movable between a compressed configuration and an extended configuration. The compressed configuration can reduce the length of the telescopic articulation device or steer the telescopic articulation device. In some implementations, the catheter has a first steering system and a second steering system. The first steering system steers the distal portion of the shaft in a first direction and a second direction. The second steering system steers the distal portion of the shaft in a third direction that is different from the first direction and the second direction. Tension is applied to the one or more actuation elements to manipulate the catheter.

### CROSS REFERENCE TO RELATED APPLICATIONS

This application is a continuation of International Application No. PCT/IB2022/061743, filed on Dec. 3, 2022, which claims the benefit of U.S. Patent Application No. 63/288,506, filed on Dec. 10, 2022, the entire disclosures all of which are incorporated by reference for all purposes.

### BACKGROUND

Endovascular delivery systems may be used in various procedures to deliver medical devices or instruments to a target location inside a patient's body that are not readily accessible by surgery or where access without surgery is desirable. The systems described herein may be used to deliver medical devices (stents, heart valve, grafts, clips, repair devices, valve treatment devices, etc.) to a location in a patient's body.

Access to a target location inside the patient's body may be achieved by inserting and guiding the delivery system through a pathway or lumen in the body, including, but not limited to, a blood vessel, an esophagus, a trachea, any portion of the gastrointestinal tract, a lymphatic vessel, to name a few. Catheters are known in the art and have been commonly used to reach target locations inside a patient's body.

In some procedures, a catheter is used to deliver a device for replacing, repairing and/or remodeling a native heart valve. The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, by congenital malformations, inflammatory processes, infectious conditions, or disease. Such damage to the valves may result in serious cardiovascular compromise or death. For many years the definitive treatment for such damaged valves was surgical repair or replacement of the valve during open heart surgery. However, open heart surgeries are highly invasive and are prone to many complications. More recently, transvascular techniques have been developed for introducing and implanting prosthetic devices in a manner that is much less invasive than open heart surgery. Transvascular techniques may be used for accessing the native mitral, aortic, tricuspid, and pulmonary valves.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The native tricuspid valve of the human heart connects the right atrium to the right ventricle. When operating properly, the leaflets of each heart valve function together as a one-way valve.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Disclosed herein are systems, devices, apparatuses, delivery systems, steerable catheters, and related methods which can be used to deliver a medical device, tools, agents, or other therapy to a location within a body of a subject. In some implementations, systems, apparatuses, devices, etc. (e.g., delivery systems or steerable catheter devices) herein can be used to deliver a medical device through the vasculature (e.g., transvascularly, transluminally, etc.), such as to a heart of the subject. For example, a flexible delivery catheter can be used to operate and/or deploy valve repair and/or replacement devices at a site for the repair or replacement of poorly functioning native heart valves.

In some implementations, a system or delivery system for delivering a medical device (such as a replacement valve, a valve repair device, a valve remodeling device, repair device, etc.) to a desired location is configured to be steered in a desired direction. The delivery system includes a catheter assembly.

In some implementations, the catheter assembly includes a catheter, a telescopic articulation device, and at least one actuation element. In some implementations, the catheter has a shaft extending from a proximal end to a distal end. In some implementations, the telescopic articulation device is disposed at the distal end of the catheter. In some implementations, the telescoping articulation device is movable between a compressed configuration and an extended configuration. In some implementations, the telescopic articulation device is steerable.

In some implementations, a catheter assembly for delivering a medical device to a desired location includes a catheter, a telescopic articulation device and at least one actuation element. The catheter comprises a shaft extending from a proximal end to a distal end. The telescopic articulation device is disposed at the distal end of the shaft. The telescoping articulation device is movable between a compressed configuration and an extended configuration. The at least one actuation element extends through the catheter. The at least one actuation element has a distal end that is attached to the telescopic articulation device. Tension applied to the at least one actuation element steers the telescopic articulation device.

In some implementations, the telescopic articulation device includes a first rigid ring, a second rigid ring, and a flexible hinge. The first rigid ring fits concentrically within the second rigid ring. The flexible hinge portion extends from a distal end of the first rigid ring to a proximal end of the second rigid ring.

In some implementations, the flexible hinge portion fits concentrically between the first rigid ring and the second rigid ring when the telescopic articulation device is in the compressed configuration.

In some implementations, the telescopic articulation device further comprises a third rigid ring and a second flexible hinge portion. In some implementations, the first rigid ring and the second rigid ring fit concentrically within the third rigid ring. In some implementations, the second flexible hinge portion extends from a distal end of the second rigid ring to a proximal end of the third rigid ring.

In some implementations, the second flexible hinge portion fits concentrically between the second rigid ring and the third rigid ring when the telescopic articulation device is in the compressed configuration.

In some implementations, the telescopic articulation device comprises a plurality of rigid rings and a plurality of flexible hinge portions. In some implementations, each of the plurality of rigid rings fits concentrically within a subsequent rigid ring of the plurality of rigid rings. In some implementations, the plurality of flexible hinge portions extend between adjacent rigid rings of the plurality of rigid rings.

In some implementations, each flexible hinge portion fits concentrically between the adjacent rigid rings when the telescopic articulation device is in the compressed configuration.

In some implementations a proximal connector is attached to a distal end of the catheter.

In some implementations, the proximal connector is attached to and arranged concentrically within the first rigid ring.

In some implementations, a fitting secures the proximal connector to the catheter.

In some implementations, the telescopic articulation device further comprises a distal connector for attaching to a second telescopic articulation device.

In some implementations, a pushing member extends the telescopic articulation device from the compressed configuration to the extended configuration.

In some implementations, the pushing member extends through a proximal end of the telescopic articulation device and is attached to a distal end of the telescopic articulation device.

In some implementations, a biasing member for extends the telescopic articulation device from the compressed configuration to an extended condition.

In some implementations, the biasing member extends between a proximal end of the telescopic articulation device to a distal end of the telescopic articulation device.

In some implementations, a system or delivery system for delivering a medical device to a desired location includes a handle, a catheter, a telescopic articulation device, a steering system, a first actuation element, and/or a second actuation element. In some implementations, the catheter comprises a shaft extending from a proximal end that is attached to the handle to a distal end.

In some implementations, the telescopic articulation device is disposed at the distal end of the shaft. In some implementations, the telescoping articulation device is movable between a compressed configuration and an extended configuration. In some implementations, the steering system is attached to the handle for steering the telescoping articulation device. In some implementations, the first actuation element extends from a proximal end that is attached to the handle to a distal end that is attached to the telescopic articulation device.

In some implementations, a second actuation element extends from a proximal end that is attached to the handle to a distal end that is attached to the telescopic articulation device.

In some implementations, tension applied to one or more of the first and second actuation elements steers the telescopic articulation device.

In some implementations, a system or delivery system for delivering a medical device to a desired location includes a handle, a catheter, a first steering system, and a second steering system. The handle comprises a housing. The catheter comprises a shaft extending from a proximal end that is attached to the handle to a distal end. In some implementations, a distal fitting is attached to the distal end. In some implementations, the first steering system is attached to the handle for steering the distal end of the shaft in a first direction and a second direction.

In some implementations, the first steering system comprises a first control member for rotating a hub and an actuation element. The actuation element extends from a first end that is attached to the hub, through the shaft, through the distal fitting, through the shaft, and to a second end that is attached to the hub. In some implementations, the second steering system is attached to the handle for steering the distal end of the shaft in a third direction that is different from the first direction and the second direction. In some implementations, the second steering system comprises a second control member for moving an actuation slider to engage the proximal end of the shaft.

In some implementations, rotating the first control member in a first control direction steers the distal end of the shaft in the first direction and rotating the first control member in a second control direction steers the distal end of the shaft in the second direction. In some implementations, actuating the second control member steers the distal end of the shaft in the third direction.

In some implementations, the second direction is opposite the first direction.

In some implementations, the shaft comprises a central lumen and a wall. In some implementations, the wall comprises a first lumen and a second lumen for the actuation element. In some implementations, the distal fitting comprises a connecting groove that connects the first lumen to the second lumen.

In some implementations, the connecting groove extends around an outer diameter of the distal fitting.

In some implementations, a first grasping element attaches a first end of the actuation element to the hub and a second grasping element attaches a second end of the actuation element to the hub.

In some implementations, the hub comprises a moveable locking portion and the housing comprises a fixed locking portion.

In some implementations, a biasing member, such as a spring, biases the hub toward the housing to cause the moveable locking portion to engage the fixed locking portion.

In some implementations, actuating the first control member in a non-rotating direction disengages the moveable locking portion from the fixed locking portion.

In some implementations, actuating the second control member causes the actuation slider to compress the shaft of the catheter against the distal fitting.

In some implementations, the third direction is oriented away from a center of the shaft toward the connecting groove of the distal fitting.

In some implementations, the second control member is a nut that is threaded onto a threaded neck extending from the housing.

In some implementations, the second control member engages a driving pin extending through the actuation slider.

In some implementations, the driving pin extends through a slot in the threaded neck.

In some implementations, moving the second control member in a proximal direction releases pressure against the proximal end of the shaft of the catheter.

In some implementations, the actuation slider is moved in the proximal direction by the shaft of the catheter.

In some implementations, the actuation element has a fixed length between the first end and the second end.

In some implementations, the connecting groove of the distal fitting comprises a securing portion and a ferrule is attached to the actuation element at the securing portion of the connecting groove when the shaft is in a straight condition.

In some implementations, a system or delivery system (e.g., a system for delivering a medical device to a desired location) comprises a handle comprising a housing, a catheter shaft extending from a proximal portion that is attached to the handle to a distal portion, wherein a distal fitting is attached to the distal portion of the catheter shaft, and a first steering system attached to the handle and for steering the distal portion of the shaft in a first direction and a second direction.

In some implementations, the system or delivery system also includes a second steering system attached to the handle and configured for steering the distal portion of the shaft in a third direction that is different from the first direction and the second direction.

In some implementations, the first steering system is configured such that actuating the first steering system in a first control direction steers the distal portion of the shaft in the first direction and actuating the first steering system in a second control direction steers the distal portion of the shaft in the second direction; and

In some implementations, the second steering system is configured such that actuating the second steering system steers the distal portion of the shaft in the third direction.

In some implementations, the first steering system comprises a first control member for actuating an actuation element extending from a first portion, which is operatively coupled to the control member, through the shaft to a second portion, which is coupled to the distal fitting.

In some implementations, a first grasping element helps operatively couple the first end of the actuation element to the control member.

In some implementations, the first steering system comprises a first control member for rotating a hub and an actuation element extending from a first portion that is attached to the hub, through the shaft, through the distal fitting, back through the shaft, and to a second portion that is attached to the hub.

In some implementations, a first grasping element attaches a first end of the actuation element to the hub and a second grasping element attaches a second end of the actuation element to the hub.

In some implementations, the hub comprises a moveable locking portion and the housing comprises a fixed locking portion. In some implementations, a biasing member biases the hub toward the housing to cause the moveable locking portion to engage the fixed locking portion.

In some implementations, the first steering system is configured such that actuating the first control member in a non-rotating direction disengages the moveable locking portion from the fixed locking portion.

In some implementations, the second steering system comprises a second control member for moving an actuation element to move the distal portion of the shaft in the third direction. In some implementations, the actuation element is an actuation slider.

In some implementations, the second steering system comprises a second control member for moving an actuation slider to move the distal portion of the shaft in the third direction.

In some implementations, the second control member is operatively coupled to the actuation element and the actuation element is coupled to the distal fitting.

In some implementations, the second control member comprises a nut that is threaded onto a threaded neck extending from the housing.

In some implementations, the second control member engages a driving pin extending through the actuation element and/or an actuation slider.

In some implementations, the driving pin extends through a slot in the threaded neck.

In some implementations, the second direction is opposite the first direction.

In some implementations, the shaft comprises a central lumen and a wall, the wall comprising a first lumen and a second lumen for the actuation element, and wherein the distal fitting comprises a connecting groove that connects the first lumen to the second lumen.

In some implementations, the connecting groove extends around an outer diameter of the distal fitting.

In some implementations, a first grasping element helps operatively couple the first end of an actuation element to the control member, and a second grasping element helps operatively couple a second end of the actuation element to the control member.

In some implementations, the second steering system is configured such that actuating the second steering system causes an actuation element to compress the shaft of the catheter against the distal fitting.

In some implementations, the third direction is oriented away from a center of the shaft toward a connecting groove of the distal fitting.

In some implementations, the second steering system is configured such that actuating the second steering system in a proximal direction releases pressure against the proximal portion of the shaft of the catheter.

In some implementations, an actuation slider is moved in the proximal direction by the shaft of the catheter.

In some implementations, the first steering system comprises a first control member for actuating an actuation element, wherein the actuation element extends from a first portion thereof, which is operatively coupled to the control member, through the shaft, to the distal fitting, then back through the shaft, and to a second portion of the actuation element that is also operatively coupled to the first control member.

In some implementations, the actuation element that has a fixed length between the first portion and the second portion.

In some implementations, a connecting groove of the distal fitting comprises a securing portion and a ferrule is attached to the actuation element at the securing portion of the connecting groove when the shaft is in a straight condition.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 is another cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIG. 8 is a perspective view of an example of a hypotube that provides structure and control of a flex section at a distal portion of a steerable catheter;

FIG. 9 is a schematic perspective cross-sectional view of the hypotube of FIG. 8;

FIG. 10 is a schematic front view of an example of a hypotube that forms a backbone of at least a part of a steerable catheter or catheter shaft;

FIG. 11 is an end view of the hypotube of FIG. 10;

FIG. 12 is a schematic cross-sectional view of the hypotube of FIG. 10;

FIG. 13 illustrates 3-dimensional steering of an example of a catheter including the example hypotube of FIGS. 10-11;

FIG. 14 is a front view of an example of a distal end portion of a steerable catheter;

FIG. 15 is a side view of the distal end portion of the steerable catheter of FIG. 14;

FIG. 16 is a cross-sectional view of the distal end portion of the steerable catheter of FIG. 14 taken along plane 29 of FIG. 15;

FIG. 17 is an enlarged view of the area 30 of FIG. 16;

FIG. 18 is a perspective view of the distal end portion of the steerable catheter of FIG. 14 with most of the springs and actuation elements removed;

FIG. 19 shows a side view of an example steering system for an example steerable catheter or delivery system;

FIG. 20 shows a side view of the example steering system for the example steerable catheter or delivery system of FIG. 19;

FIG. 21 shows a side view of the example steering system for the example steerable catheter or delivery system of FIG. 19;

FIG. 22 shows a side view of the example steering system for the example steerable catheter or delivery system of FIG. 19;

FIG. 23 shows a side view of an example steering system for an example steerable catheter or delivery system;

FIG. 24 shows a front view of the example steering system for the example steerable catheter or delivery system of FIG. 23;

FIG. 25 shows a side view of the example steering system for the example steerable catheter or delivery system of FIG. 23;

FIG. 26 shows a front view of the example steering system for the example steerable catheter or delivery system shown in FIG. 25;

FIG. 27 shows a perspective view of the example steering system for the example steerable catheter or delivery system shown in FIG. 25;

FIG. 28 shows a side view of an example steering system for an example steerable catheter or delivery system;

FIG. 29 shows a front view of the example steering system for the example steerable catheter or delivery system of FIG. 28;

FIG. 30 shows a side view of the example steering system for the example steerable catheter or delivery system of FIG. 28;

FIG. 31 shows a front view of the example steering system for the example steerable catheter or delivery system shown in FIG. 30;

FIG. 32 shows a perspective view of a rack from the example steering system for the example steerable catheter or delivery system of FIG. 28;

FIG. 33 shows a perspective view of a worm gear the example steering system for the example steerable catheter or delivery system of FIG. 28;

FIG. 34 shows a schematic cross-sectional view of an example steering system for an example steerable catheter or delivery system;

FIG. 35 shows a schematic front view of the example steering system for the example steerable catheter or delivery system of FIG. 34;

FIG. 36 shows a schematic cross-sectional view of an example steering system for an example steerable catheter or delivery system;

FIG. 37 shows a schematic back view of an example handle of an example steering system for an example steerable catheter or delivery system;

FIG. 38 shows a schematic side view of the example handle of an example steering system for the example steerable catheter or delivery system of FIG. 37;

FIG. 39 shows a cross-sectional view of the example handle of an example steering system for the example steerable catheter or delivery system of FIG. 37;

FIG. 40 shows a schematic side view of an example handle of an example steering system for an example steerable catheter or delivery system;

FIG. 41 shows a schematic front view of the example handle of an example steering system for the example steerable catheter or delivery system of FIG. 40;

FIG. 42 shows a perspective view of an example grasping element for retaining an actuation element for an example steerable catheter or delivery system;

FIG. 43 shows a cross-sectional view of the example grasping element for retaining an actuation element for the example steerable catheter or delivery system of FIG. 42;

FIG. 44 shows a partial front view of a steering system for an example steerable catheter or delivery system including an example grasping element for retaining an actuation element;

FIG. 45 shows a cross-sectional view of the steering system including an example grasping element for retaining an actuation element of FIG. 44 taken along the line A-A;

FIG. 46 shows a cross-sectional view of the steering system of FIG. 44 taken along the line A-A without the example grasping element for retaining an actuation element;

FIG. 47 shows a perspective view of the grasping element for retaining an actuation element of FIG. 44;

FIG. 48 shows a schematic cross-sectional view of an example telescoping tube structure for actuation elements of a steering system for an example steerable catheter or delivery system;

FIG. 49 illustrates 3-dimensional steering of an example of a catheter including an example steering system;

FIG. 50 shows a front view of an example control handle including a steering system for steering of steerable catheter or catheter shaft;

FIG. 51 shows a perspective view of an example steering system for an example steerable catheter or delivery system;

FIG. 52 shows a front view of the example steering system of FIG. 51;

FIG. 53 shows a cross-sectional perspective view of the example steering system of FIG. 51;

FIG. 54 shows a cross-sectional front view of the example steering system of FIG. 51;

FIGS. 55-56 show a schematic front view of an example steering system;

FIG. 57 shows a perspective view of an example grasping element for retaining an actuation element for a steerable catheter or delivery system;

FIG. 58 shows a cross-sectional view of the example grasping element for retaining an actuation element for the steerable catheter or delivery system of FIG. 57;

FIG. 59 shows a perspective view of an example grasping element for retaining an actuation element for a steerable catheter or delivery system with the actuation element in a retracted position;

FIG. 60 shows a cross-sectional view of the example grasping element for retaining an actuation element for the steerable catheter or delivery system of FIG. 59 with the actuation element in a retracted position;

FIG. 61 shows a schematic view of an example grasping element for retaining an actuation element for a steerable catheter or delivery system;

FIG. 62 shows a schematic view of an example grasping element for retaining an actuation element for a steerable catheter or delivery system;

FIG. 63 shows a schematic view of an example grasping element for retaining an actuation element for an example steerable catheter or delivery system;

FIG. 64 shows a schematic view of an example steering system for an example steerable catheter or delivery system;

FIG. 65 shows a schematic view of an example steering system for an example steerable catheter or delivery system;

FIGS. 66-67 show perspective views of an example control handle with an example steering system for an example steerable catheter or delivery system;

FIG. 68 shows a perspective cross-sectional view of the example control handle with an example steering system for the steerable catheter or delivery system of FIG. 66;

FIG. 69 shows a front cross-sectional view of the example control handle with an example steering system for the steerable catheter or delivery system of FIG. 66;

FIG. 70 shows a perspective view of the example steering system of the example control handle of FIG. 66;

FIG. 71 shows a front view of the example steering system of FIG. 70;

FIG. 72 shows a top view of the example steering system of FIG. 70;

FIG. 73 shows a side view of the example steering system of FIG. 70;

FIG. 74 shows a perspective view of an example telescopic articulation device attached to a distal end of a catheter with the example telescopic articulation device in a collapsed condition;

FIG. 75 shows a side view of the example telescopic articulation device of FIG. 74;

FIG. 76 shows a perspective cross-sectional view of the example telescopic articulation device of FIG. 74 taken along the line 75-75 of FIG. 75;

FIG. 77 shows a cross-sectional view of the example telescopic articulation device of FIG. 74 taken along the line 75-75 of FIG. 75;

FIG. 78 shows an enlarged detail view of the area 76 of FIG. 76;

FIG. 79 shows an enlarged detail view of the area 77 of FIG. 77;

FIG. 80 shows a perspective exploded view of the example telescopic articulation device and catheter of FIG. 74;

FIG. 81 shows a front view of the fitting for connecting the example telescopic articulation device and the catheter of FIG. 80;

FIG. 82 shows a side view of the fitting of FIG. 81;

FIG. 83 shows a cross-sectional view of the fitting of FIG. 81 taken along the line 82-82 of FIG. 82;

FIG. 84 shows a perspective cross-sectional view of the fitting of FIG. 81 taken along the line 82-82 of FIG. 82;

FIG. 85 shows a perspective view of an example telescopic articulation device attached to a distal end of a catheter with the example telescopic articulation device in a partially extended condition;

FIG. 86 shows a side view of the example telescopic articulation device of FIG. 85;

FIG. 87 shows a perspective cross-sectional view of the example telescopic articulation device of FIG. 85 taken along the line 86-86 in FIG. 86;

FIG. 88 shows a cross-sectional view of the example telescopic articulation device of FIG. 85 taken along the line 86-86 in FIG. 86;

FIG. 89 shows an enlarged detail view of the area 87 of FIG. 87;

FIG. 90 shows an enlarged detail view of the area 88 of FIG. 88;

FIG. 91 shows a cross-sectional view of the example telescopic articulation device of FIG. 85 with the example telescopic articulation device in a partially bent condition;

FIG. 92 shows a perspective view of an example telescopic articulation device attached to a distal end of a catheter with the example telescopic articulation device in a fully extended condition;

FIG. 93 shows a side view of the example telescopic articulation device of FIG. 92;

FIG. 94 shows a perspective cross-sectional view of the example telescopic articulation device of FIG. 92 taken along the line 93-93 of FIG. 93;

FIG. 95 shows a cross-sectional view of the example telescopic articulation device of FIG. 92 taken along the line 93-93 of FIG. 93;

FIG. 96 shows an enlarged detail view of the area 94 of FIG. 94;

FIG. 97 shows an enlarged detail view of the area 95 of FIG. 95;

FIG. 98 shows a cross-sectional view of the example telescopic articulation device of FIG. 92 with the example telescopic articulation device in a partially bent condition;

FIG. 99 shows a cross-sectional view of the example telescopic articulation device of FIG. 92 with the example telescopic articulation device in a fully bent condition;

FIG. 100 shows a perspective view of three example telescopic articulation devices attached to a distal end of a catheter with each of the example telescopic articulation devices in a collapsed condition;

FIG. 101 shows a side view of the example telescopic articulation devices of FIG. 100;

FIG. 102 shows a perspective cross-sectional view of the example telescopic articulation devices of FIG. 100 taken along the line 101-101 of FIG. 101;

FIG. 103 shows a cross-sectional view of the example telescopic articulation devices of FIG. 100 taken along the line 101-101 of FIG. 101;

FIG. 104 shows an enlarged detail view of the area 102 of FIG. 102;

FIG. 105 shows an enlarged detail view of the area 103 of FIG. 103;

FIG. 106 shows a perspective view of three example telescopic articulation devices attached to a distal end of a catheter with each of the example telescopic articulation devices in a fully extended condition;

FIG. 107 shows a side view of the example telescopic articulation device of FIG. 106;

FIG. 108 shows a perspective cross-sectional view of the example telescopic articulation device of FIG. 106 taken along the line 107-107 of FIG. 107;

FIG. 109 shows a cross-sectional view of the example telescopic articulation device of FIG. 106 taken along the line 107-107 of FIG. 107;

FIG. 110 shows an enlarged detail view of the area 108 of FIG. 108;

FIG. 111 shows an enlarged detail view of the area 109 of FIG. 109;

FIG. 112 shows a perspective cross-sectional view of an example telescopic articulation device in an expanded condition and including a biasing member;

FIG. 113 shows a cross-sectional view of the example telescopic articulation device of FIG. 112;

FIG. 114 shows a perspective cross-sectional view of an example telescopic articulation device in an expanded condition and including a pushing member;

FIG. 115 shows a cross-sectional view of the example telescopic articulation device of FIG. 114;

FIG. 116 shows a top perspective view of an example control handle for a delivery system;

FIG. 117 shows a bottom perspective view of the example control handle of FIG. 116;

FIG. 118 shows a side cross-sectional view of the example control handle of FIG. 116;

FIG. 119 shows a top cross-sectional view of the example control handle of FIG. 116;

FIG. 120 shows a top perspective exploded view of the example control handle of FIG. 116;

FIG. 121 shows a bottom perspective exploded view of the example control handle of FIG. 116;

FIG. 122 shows an enlarged view of an example distal end of the catheter shaft useable with the example control handle of FIG. 116;

FIG. 123 shows an enlarged cross-sectional view of the distal end of the catheter shaft of FIG. 122;

FIG. 124 shows a bottom perspective view of example first and second steering systems of the example control handle of FIG. 116;

FIG. 125 shows an enlarged view of an example second steering system of the example control handle of FIG. 116; and

FIG. 126 shows a side cross-sectional view of an example of a control handle that is configured to allow another catheter to pass through the control handle and attached catheter.

### DETAILED DESCRIPTION

The following description refers to the accompanying drawings, which illustrate example implementations of the present disclosure. The drawings demonstrate several possible configurations of systems, devices, components, and methods that can be used for various aspects and features of the present disclosure. Other implementations having different structures and operation do not depart from the scope of the present disclosure. Specific examples provided herein are not intended to be limiting; for example, steering systems or steering mechanisms described herein can also be adapted and used to steer other systems and devices not expressly described herein. As one example, various systems, devices, components, and methods are described herein that may relate to steerable delivery systems or steerable catheters. As a further example, Published PCT Patent Application No. WO2020/106705, which is incorporated by reference herein in its entirety, also describes various delivery systems or steerable catheters that can be used with the steering systems, steering mechanisms, steering elements, and other features described herein.

Example implementations of the present disclosure are directed to systems, devices, methods, delivery systems, steering systems, etc. useable for repairing a heart valve and/or delivering treatment or repair systems, devices, apparatuses, etc. to repair and/or replace a heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the treatment techniques, methods, and steps herein can be performed on a living animal or on a simulation, such as on a cadaver, cadaver heart, simulator (e.g., with the body parts, heart, tissue, etc. being simulated), etc.

Some example systems or delivery systems herein provide a wide range of motion for the positioning of a medical device and are versatile, reliable, and easy to use. For example, the delivery systems disclosed herein can be used to position and deploy an implantable medical device for use in the repair of a heart valve

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

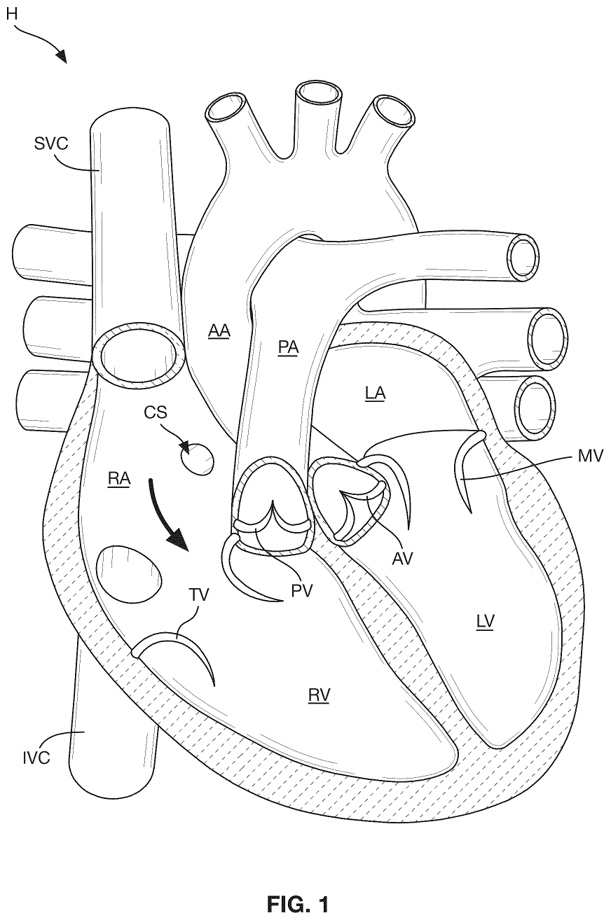

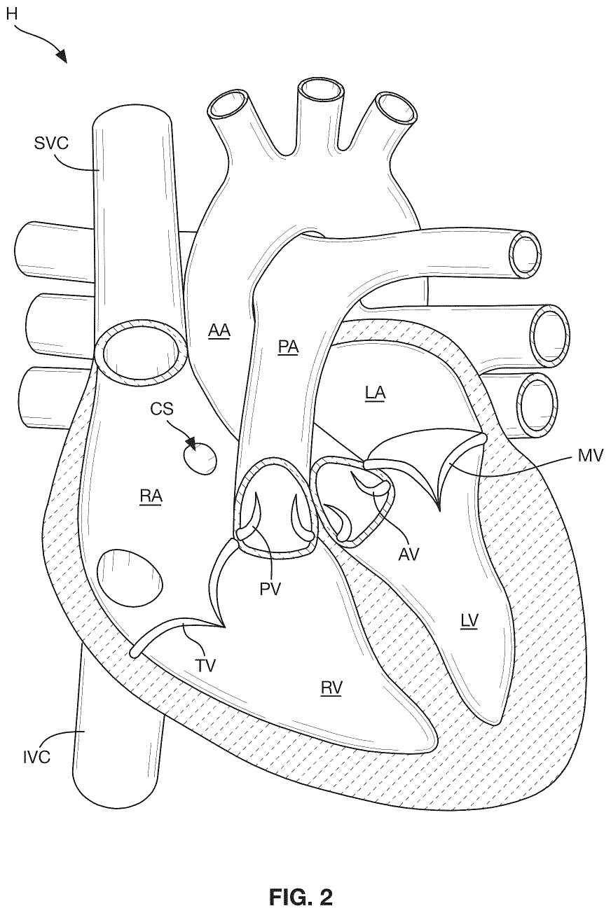

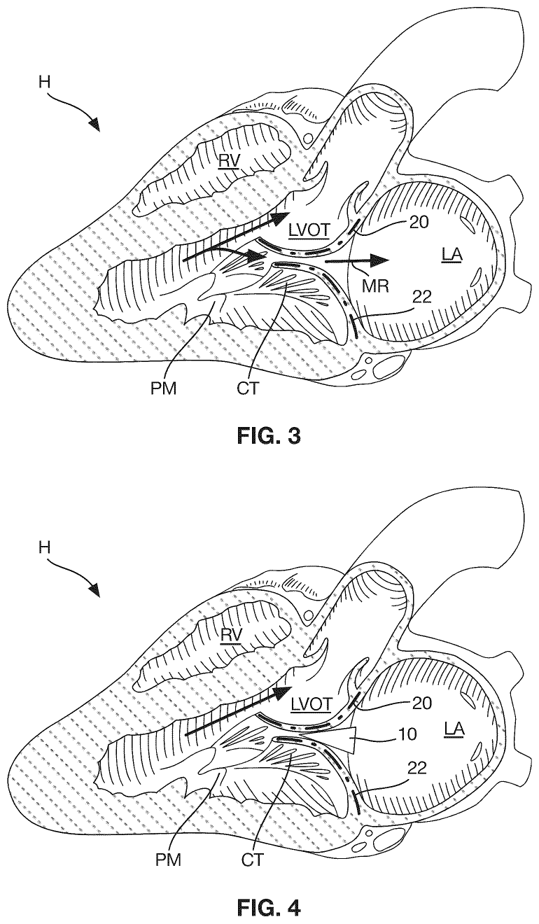

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV, respectively; i.e., the atrioventricular valves. The aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-7) extending inward across the respective orifices that come together or “coapt” in the flow stream to form the one-way, fluid-occluding surfaces. The devices and systems disclosed herein can be used to replace, repair, remodel, etc. the mitral valve MV, the tricuspid valve TV, the aortic valve AV, and/or the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV back into the left atrium LA and blood is collected in the left atrium from the pulmonary vein.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIG. 3, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV to prevent the mitral valve MV from being reverted. The mitral valve MV opens and closes in response to relative pressure changes in the left atrium LA and the left ventricle LV. The papillary muscles PM do not open or close the mitral valve MV. Rather, the papillary muscles PM support or brace the leaflets 20, 22 against the high pressure necessary to circulate blood throughout the body. Together the papillary muscles PM and the chordae tendineae CT are known as the subvalvular apparatus, which functions to keep the mitral valve MV from prolapsing into the left atrium LA when the mitral valve closes.

As seen from a Left Ventricular Outflow Tract (LVOT) view shown in FIG. 4, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus. As a result, the leaflets 20, 22 form a space having a generally triangular shape 10 that is annotated in FIG. 4.

Various disease processes can impair proper function of one or more of the native valves of the heart H. These disease processes include degenerative processes (e.g., Barlow's Disease, fibroelastic deficiency), inflammatory processes (e.g., rheumatic heart disease), and infectious processes (e.g., endocarditis). In addition, damage to the left ventricle LV or the right ventricle RV from prior heart attacks (i.e., myocardial infarction secondary to coronary artery disease) or other heart diseases (e.g., cardiomyopathy) can distort a native valve's geometry, which can cause the native valve to dysfunction.

Generally, a native valve may malfunction in two different ways: (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. The second type of valve malfunction, valve regurgitation, occurs when the leaflets of the valve do not close completely thereby causing blood to leak back into the prior chamber (e.g., causing blood to leak from the left ventricle to the left atrium).

There are three mechanisms by which a native valve becomes regurgitant—or incompetent-which include Carpentier's type I, type II, and type III malfunctions. A Carpentier type I malfunction involves the dilation of the annulus such that normally functioning leaflets are distracted from each other and fail to form a tight seal—i.e., the leaflets do not coapt properly. Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

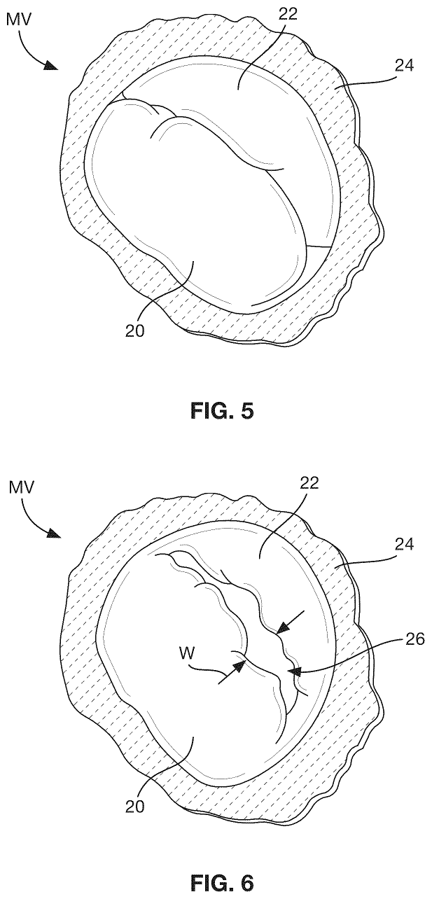

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22 that allows blood to flow back into the left atrium LA from the left ventricle LV during systole, as illustrated by the mitral regurgitation MR flow path shown in FIG. 6. As set forth above, there are several different ways that a leaflet (e.g., leaflets 20, 22 of mitral valve MV) may malfunction such that mitral regurgitation MR occurs.

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart is primarily responsible for circulating the flow of blood throughout the body, substantially higher pressures are experienced by the left side heart structures (i.e., the left atrium LA, the left ventricle LV, the mitral valve MV, and the aortic valve AV). Accordingly, malfunction of the mitral valve MV or the aortic valve AV is particularly problematic and often life threatening.

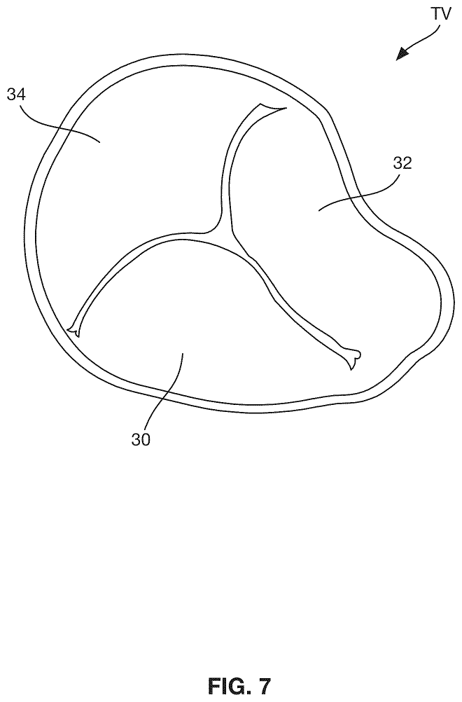

Malfunctioning native heart valves may either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, the most conventional treatments for a stenotic aortic valve or stenotic pulmonary valve are removal and replacement of the valve with a surgically implanted heart valve, or displacement of the valve with a transcatheter heart valve. The mitral valve MV and the tricuspid valve TV (FIG. 7) are more prone to deformation of annulus and/or leaflets, which, as described above, prevents the mitral valve MV or tricuspid valve TV from closing properly and allows for regurgitation or back flow of blood from the ventricle into the atrium (e.g., a deformed mitral valve MV may allow for mitral regurgitation MR or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV.

The devices and concepts provided herein can be used to replace any native valve, repair any native valve, remodel any native valve, as well as any component of a native valve. In one non-limiting example, a valve repair device can be used on native mitral leaflets 20, 22 or tricuspid leaflets 30, 32, 34.

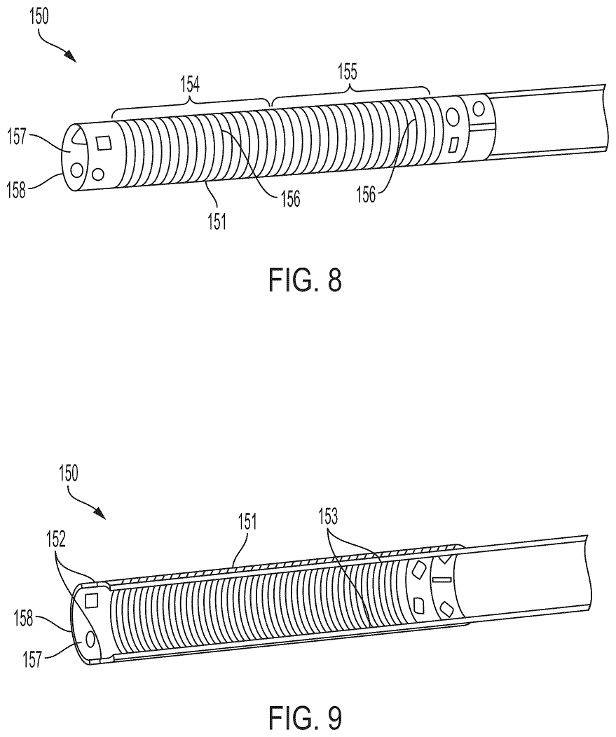

Referring now to FIGS. 8-9, an example steerable catheter 150 is shown that includes a hypotube 151 arranged at a distal portion of the catheter 150 to provide structure and control of the flex section at the distal portion of the catheter 150. The catheter 150 can include at least one shaft that includes the hypotube 151 and relies on the hypotube 151 as a backbone or spine of the entire shaft. The hypotube 151 can be co-centric with the rest of the catheter shaft. And can extend for the entire length or substantially the entire length of the catheter shaft. Optionally, the hypotube 151 can be located only at a distal portion or distal end of the shaft. A distal end 158 of the hypotube 151 can be aligned with or spaced apart from the distal portion or end of the catheter 150.

The hypotubes described herein, such as, for example, the hypotube 151, can be constructed using any suitable metal or alloy, such as, for example, stainless steel, nitinol, titanium, and the like. The hypotube 151 can also include an internal liner made from a material having substantially same properties as the material used to make lumens used in conjunction with other structures of the catheter, such as actuation elements (e.g., control wires, pull wires, lines, sutures, wires, rods, etc.) or compression members (e.g., compression coils, compression springs, compression tubes, etc.). Additionally, a reflowed jacket or outer material can be provided over the entirety of the catheter shaft.

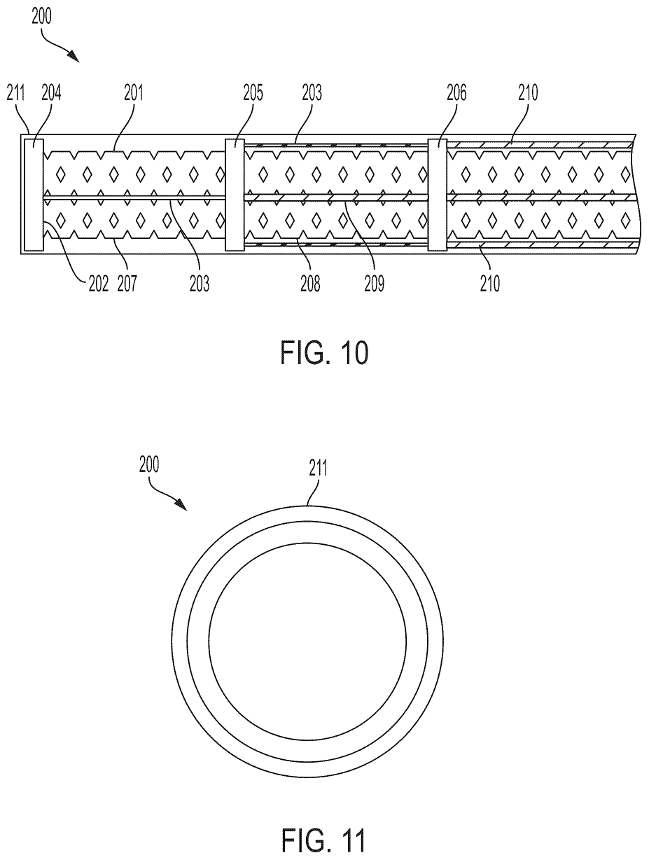

An operator of the catheter 150 can bend the distal portion of the catheter 150 by pulling or releasing actuation elements 153 (e.g., control wires, pull wires, lines, sutures, wires, rods, etc.) that extend along the length of the hypotube 151. The actuation elements 153 can be pull wires having a circular or flat rectangular cross-sectional shape and can be secured to the hypotube 151 via a pull ring (see, e.g., FIG. 10) or via attachment locations 152. The actuation elements 153 can be directly attached to the attachment locations via welding, an adhesive, a mechanical fastener, or the like. For example, the actuation elements 153 can be laser welded to the attachment locations 152 of the hypotube 151.

A first or anchor portion 157 of the hypotube 151 proximal to the distal end 158 of the hypotube 151 can serve the same function as a pull-ring, that is, to attach to the actuation elements 153 so that tension applied to the actuation elements 153 is transmitted to the hypotube 151. Integrating a pull ring structure into the hypotube 151 prohibits misalignment of the pull ring and hypotube 151 that can occur when two separate components are joined together. The anchor portion 157 can be formed from an area of the hypotube 151 with a particular shape and/or increased strength, rigidity, and thickness to provide sufficient strength for receiving the actuation elements 153. To provide added stiffness and strength, the wall thickness of the anchor portion 157 of the hypotube 151 proximal to the distal end 158 of the hypotube 151 can be greater than the wall thickness of the remainder of the hypotube 151. For example, a thickness of a wall of an anchor portion 157 of the hypotube can be in the range of about 0.5 mm to about 2.5 mm. The anchor portion 157 of the hypotube 151 can also be wider than the remainder of the hypotube 151, the anchor portion 157 having a diameter in a range of about 5 mm to about 10 mm.

The hypotube 151 can be formed from a single section that bends substantially uniformly when actuated by the actuation elements 153. To facilitate bending, the hypotube 151 can include a plurality of relief cuts 156 that allow the hypotube 151 to flex and bend in one or more flexing directions with little or no axial compression under load. The relief cuts 156 can be formed in the hypotube 151 via laser cutting or any other suitable cuttings means to alter the bending characteristics of the hypotube 151. The relief cuts 156 can be formed in a variety of patterns, e.g., straight, spiral, staggered, zig-zag, etc. In some implementations, the repeating cuts 156 are aligned in a straight line along the axis of the shaft of the catheter. In some implementations, the repeating cuts 156 are staggered along the axis of the shaft of the catheter.

The hypotube 151 can include sections having different bending characteristics: that is, a first section 154 arranged near the distal end 158 of the hypotube 151 and a second section 155 proximal of the first section. That is, the first and second sections 154, 155 can differ in bend direction, bend rate, and bend radius when tension is applied to the actuation elements 153 to actuate the hypotube 151. The different bending characteristics of the first and second sections 154, 155 of the hypotube 151 can be provided in a wide variety of ways, such as, for example, by varying the thickness, stiffness, and material type of the first and second sections 154, 155.

The structure of the first and second sections 154, 155 can also be varied via the relief cuts 156 along the hypotube 151. The relief cuts 156 can be changed in their size, shape, and spacing along the length of the hypotube 151 and, in particular, between the first and second sections 154, 155 to provide different bending characteristics between the first and second sections 154, 155. For example, the bending direction can be altered between the first and second sections 154, 155. The spacing between consecutive cuts in the first section 154 can be greater than, the same as, or lesser than the spacing between consecutive cuts in the second section 155. In some implementations, the relief cuts 156 are formed such that links or link-like formations are formed in the hypotube 151.

Referring now to FIGS. 10-13, an example delivery system 200 is shown that includes a catheter or catheter shaft 211 and a hypotube 201 arranged within the catheter 211. The hypotube 201 extends along the catheter or catheter shaft 211 to a distal end 202 and can be caused to bend or flex via the actuation of a plurality of actuation elements 203 (e.g., control wires, pull wires, lines, sutures, wires, rods, etc.) arranged around the circumference of the catheter or catheter shaft. The hypotube 201 can be divided longitudinally into sections that can be independently actuated by the actuation elements 203. In some implementations, the bending characteristics from one section of the hypotube 201 to another can be different.

The hypotube 201 includes first, second, and third ring sections 204, 205, 206 that provide support to the actuation elements 203 and also attachment locations for attaching the actuation elements 203 to the hypotube 201. The first ring section 204 is arranged at the distal end 202 of a first section 207 of the hypotube 201, the second ring section 205 is arranged at a mid-section between the first section 207 and a second section 208, and the third ring section 206 is arranged at a proximal end of the second section 208. While three ring sections 204, 205, 206 are shown in FIG. 2, the hypotube can include any suitable number of ring sections, such as, for example, 2, 3, 4, 5, or more ring sections that can correspond to a similar number of articulable sections of the hypotube 201.

The hypotube 201 can be formed from a tube of material or a sheet of material that is rolled and welded or otherwise joined along a seam. The tube or sheet of material can have a plurality of spaced apart cutouts arranged in a grid and each having a diamond shape (see FIG. 10) or any other suitable shape. For example, the cutouts can be formed as slits that extend around a majority of the circumference of the hypotube 201 to form a series of links having a rib cage like configuration. In some implementations, the links include a slot formed between each pair of adjacent links, a bottom orifice for each link, and at least one slit extending upward from the bottom orifice.

The ring sections 204, 205, 206 can have an outer diameter that is the same as the outer diameter of the hypotube 201. The outer diameter of one or more of the ring sections 204, 205, 206 can also be larger than the outer diameter of the hypotube 201 and smaller than an inside diameter of the catheter shaft—i.e., the diameter of the ring sections 204, 205, 206 can be larger than the diameter of the hypotube 201 yet be small enough to fit within the catheter 211. The ring sections 204, 205, 206 can optionally be integrally formed in the hypotube 201 by cutting or otherwise forming the ring sections 204, 205, 206 in the material of the hypotube 201. Optionally, the ring sections 204, 205, 206 can be separate rings that are attached to the outer surface of the hypotube 201 via any suitable attachment means, such as, for example, welding, an adhesive, mechanical fastening, or the like.

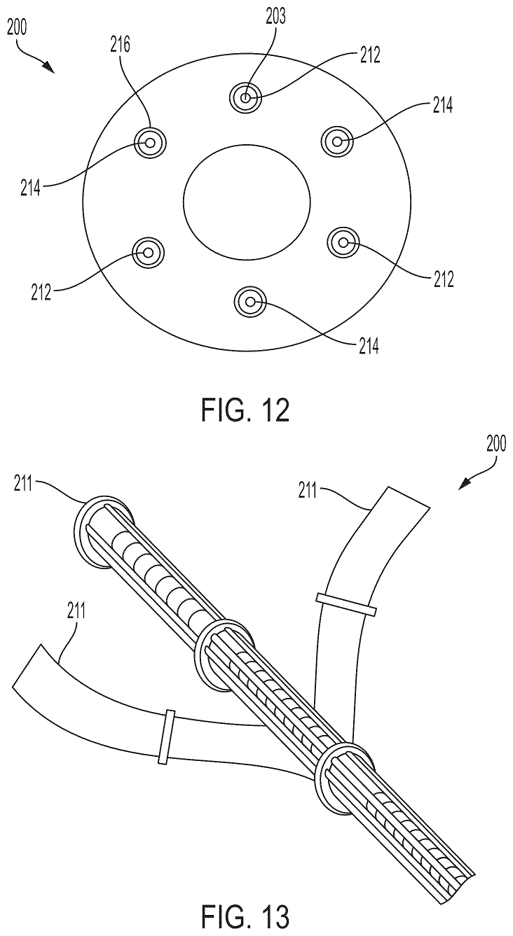

The actuation elements 203 are arranged into two groups: a first group 212 (FIG. 12) for articulating the first section 207 and a second group 214 for articulating the second section 208. The actuation elements 203 of the first group 212 extend through openings 216 in the second and third ring sections 205, 206 to attach to the first pull ring 204. The actuation elements 203 of the first group 212 extend through compression members 209 (e.g., compression coils, compression springs, compression tubes, etc.) that terminate at the second pull ring 205. The actuation elements 203 of the second group 214 extend through openings 216 in the third ring section 206 to attach to the second pull ring 205. The actuation elements 203 of the second group 214 extend through compression members 210 that terminate at the third pull ring 206.

As can be seen in FIG. 12, the actuation elements 203 in each of the first and second groups 212, 214 are radially spaced apart from each other by about 120 degrees so that the three actuation elements 203 in each group are evenly spaced around the circumference of the hypotube 201. Optionally, one of the three actuation elements 203 in the first or second groups 212, 214 can be spaced apart about 135 degrees from the other two actuation elements 203. Additional actuation elements 203 can also be included such that the actuation elements 203 are radially spaced apart from other actuation elements 203 by about 90 degrees, about 76 degrees, about 60 degrees, or about 30 degrees.

Applying tension to the actuation elements 203 causes the attached ring section 204, 205, 206 to move or tilt in the direction of the net tension force applied to the ring section 204, 205, 206. Consequently, the first or second section 207, 208 of the hypotube 201 that is immediately proximal of the articulated ring section 204, 205 is caused to flex or bend toward the applied force. Bending forces applied to one side of the hypotube 201 via one of the three actuation elements 203 in one of the first and second groups 212, 214 can be counteracted by forces applied to the other two actuation elements 203 in the first or second group 212, 214.

Providing three actuation elements 203 in each of the first and second groups 212, 214 enables each of the first and second sections 207, 208 to be articulated in any direction around the hypotube 201 by varying the amount of tension applied to each of the actuation elements 203. In particular, the direction of the bend in the hypotube 201 depends on the proportional distribution of tension forces in each of the three actuation elements 203 of the first or second groups 212, 214. That is, the relative proportion of tension applied to each of the three actuation elements 203—independent of the amount of tension applied—determines bend direction. The amount of tension applied to the actuation elements 203, however, is directly related to the magnitude of the bend in the hypotube 201; the greater the tension imbalance the greater the bend magnitude (i.e., the tighter or smaller the bend radius). Consequently, the catheter or catheter shaft 211 of the delivery system 200 can be articulated into a wide variety of positions, such as, for example, the range of positions shown in FIG. 13.

The actuation elements from each group 212, 214 can be connected to one or more steering elements of a steering system/mechanism, such as an example steering system/mechanism disclosed elsewhere herein, e.g., that is arranged in a handle (not shown) at a proximal end of the catheter or catheter shaft. For example, a first steering system/mechanism can be used to control the bending of the first section 207 and a second steering system/mechanism can be used to control the bending of the second section 208. Optionally, a single steering system/mechanism can control both of the first and second sections 207, 208. The steering systems/mechanisms can be arranged in a single handle or in multiple handles such that each handle contains a single steering system/mechanism.

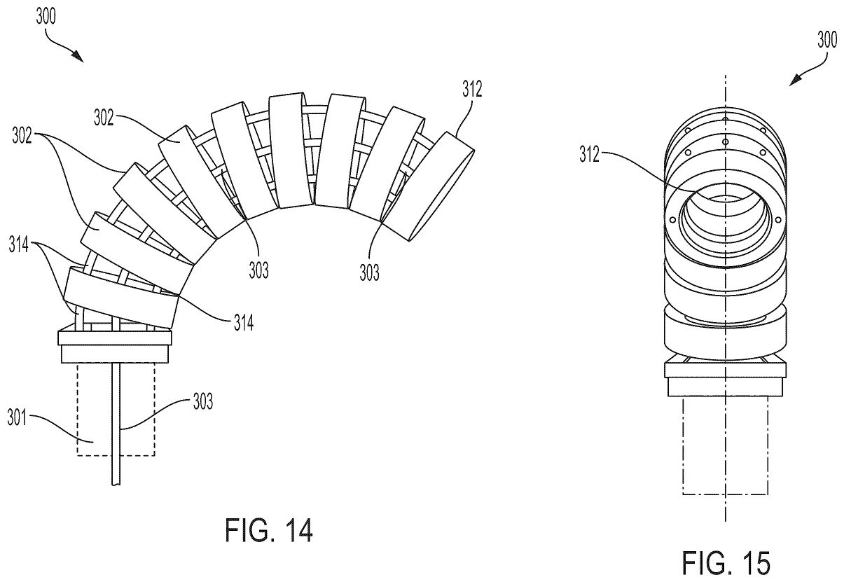

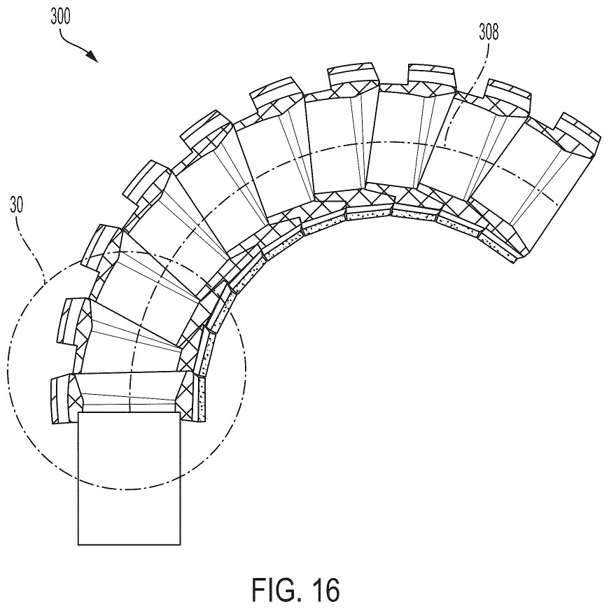

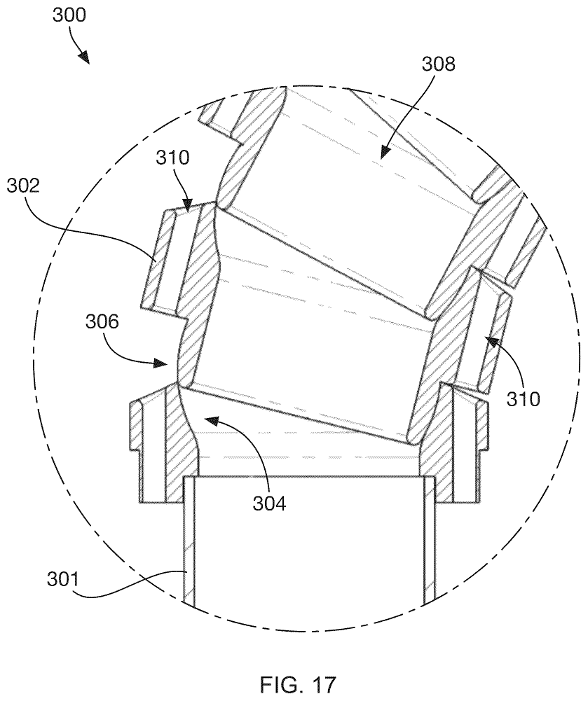

Referring now to FIGS. 14-18, an example delivery system 300 is shown. The delivery system 300 is formed from a plurality of links 302 arranged at a distal end of a catheter shaft 301. The links 302 operate similar to vertebrae of the human spine in that the links 302 include male and female connecting surfaces 304, 306 that provide a sliding joint between adjacent links 302. The connecting surfaces 304, 306 can be formed in a ball and socket joint configuration that allows the links 302 to pivot relative to one another. Each link 302 includes a central opening or lumen 308 and openings or lumen 310 for guiding and supporting actuation elements 303 (e.g., control wires, pull wires, lines, sutures, wires, rods, etc.) that extend along the length of the delivery device 300 to a distal end 312. An optional hypotube (not shown) can also be provided that extends through the central openings 308 of the links 302.

The actuation elements 303 and actuation element openings 310 are radially spaced apart from each other by about 90 degrees to accommodate four actuation elements 303 extending along the length of the device 300 to the distal end 312. Applying tension to the actuation elements 303 causes the distal end 312 and the links 302 to move or tilt in the direction of the net tension force applied to the actuation elements 303. Consequently, the plurality of links 302 are caused to pivot relative to each other so that the device 300 flexes or bends toward the applied force. Bending forces applied to one side of the device 300 via one of the three actuation elements 303 can be counteracted by forces applied to the other actuation elements 303.

Providing four actuation elements 303 around the circumference of the device 300 enables the device 300 to be articulated in any direction around by varying the amount of tension applied to each of the actuation elements 303. In particular, the direction of the bend in the device 300 depends on the proportional distribution of tension forces in each of the four actuation elements 303. That is, the relative proportion of tension applied to each of the four actuation elements 303—independent of the amount of tension applied-determines bend direction. The amount of tension applied to the actuation elements 303, however, is directly related to the magnitude of the bend in the device 300; the greater the tension imbalance the greater the bend magnitude (i.e., the tighter or smaller the bend radius). Consequently, the end of the catheter or catheter shaft 301 of the delivery system 300 can be articulated into a wide variety of positions.

The actuation elements 303 can be connected to one or more steering elements of a steering system/mechanism, such as an example steering systems or steering mechanisms disclosed elsewhere herein, e.g., that is arranged in a handle (not shown) at a proximal end of the catheter or catheter shaft. For example, a first steering system/mechanism can be used to control the bending in a first bending plane of the device 300 so that the steering system/mechanism is connected to two actuation elements 303 that are spaced apart by 180 degrees around the device 300 and a second steering system/mechanism can be used to control the bending of the device 300 in a second bending plane that is orthogonal to the first bending plane. Optionally, a single steering system/mechanism can control bending in both the first and second bending planes. The steering system/mechanisms can be arranged in a single handle or in multiple handles such that each handle contains a single steering system/mechanism.

The actuation elements 303 can extend through compression members or coils (not shown) that extend from a handle or proximate the handle to a proximal portion of the most proximal link 302. The proximal side of the link 302 can include pockets or recesses for receiving a distal end of the compression member. Optionally, the compression member can run the entire length of the catheter shaft 301. In any of the catheter implementations herein, each compression member can run through an individual lumen in a shaft of the catheter so that flexing of the shaft does not hinder independent movement of the compression member. In some implementations, the proximal face of a hypotube and/or links of a hypotube have bores and/or extensions to accept or abut against the compression members. The proximal face of the most proximal link 302 can also have bores and/or extensions to accept or abut against the compression members.

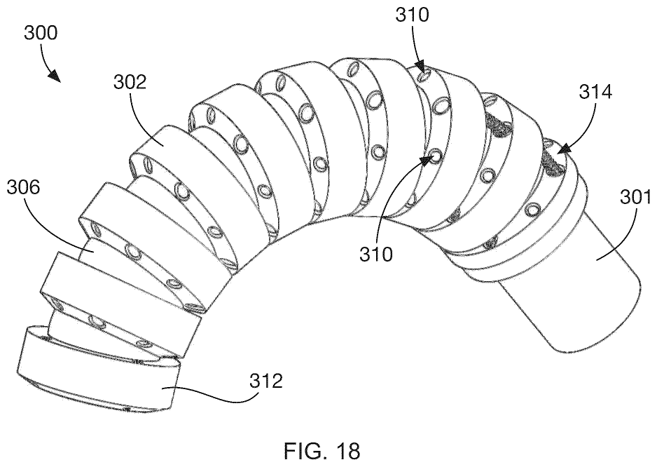

The device 300 can further include stiffening members arranged between the links 302. The stiffening members cause the device 300 to be biased in an extension direction so that the links 302 tend to straighten out after tension applied to the actuation elements 303 is relieved. The stiffening members can be formed in a tube shape from a shape-memory alloy, such as nitinol. As can be seen in FIG. 18, the stiffening members can be springs 314 that are biased in an expanding direction so that as the device 300 tends to straighten as tension applied to the actuation elements 303 is relieved. The springs 314 can be arranged between each pair of adjacent links 302 or can extend through multiple links 302. Four springs 314 can be arranged between each pair of adjacent links 302 so that the springs 314 are radially spaced apart by about 90 degrees and can be arranged between adjacent actuation elements 303 so that the springs 314 and actuation elements 303 alternate around the circumference of the device 300. Evenly spacing the springs 314 around the circumference of the links 302 evens out the forces applied to the links 302 and helps to maintain a symmetrical distance between adjacent links 302.

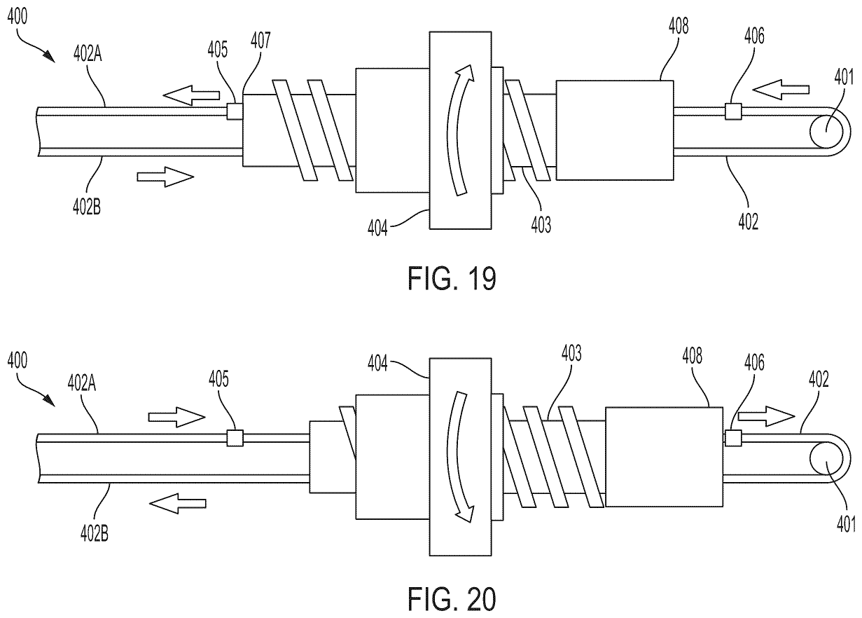

Referring now to FIGS. 19-22, an example steering system/mechanism 400 for a delivery system is shown. The steering system/mechanism 400 (e.g., assembly of components that work together to steer, etc.) can be included inside of a handle of the delivery system and can be attached to proximal end of a catheter shaft. The steering system/mechanism 400 can include a pulley 401, one or more actuation elements 402 (e.g., control wires, pull wires, lines, sutures, wires, rods, etc.), and a steering element 403. The pulley 401 can be formed as a wheel, tube, shaft, pin, or the like. The steering element 403 (and/or other steering elements herein) can comprise one or more of threaded element (e.g., a screw, worm screw, steering screw, knob, split screw, and/or the like), a translating member, control, a tube, a shaft, shuttle, and/or the like. The pully can be made from a low friction material, such as, for example, polytetrafluoroethylene (PTFE).

The actuation element 402 is routed around the pulley 401 and through the steering element 403. Each end of the actuation element 402 attaches to a distal location in the delivery system, such as, for example, a pull ring, a low-profile ring, a hypotube, or the like. Each end of the actuation element 402 can extend through a compression member (e.g., compression coils, compression springs, compression tubes, etc.) that extends for a portion of the actuation element 402 or for substantially the entire length of the actuation element 402. In some examples with two actuation elements, a first actuation element 402 can extend from a first attachment point in the delivery system, over the pulley 401, and to the steering element 403 and a second actuation element 402 can extend from a second attachment point in the delivery system to the steering element 403. The first and second actuation elements 402 can be attached to each other at the steering element 403 or can each be attached directly to the steering element 403. The actuation elements 402 can run parallel to each other through a lumen in a catheter shaft of the delivery system and can be partially or fully surrounded by compression members. The actuation elements 402 can be spaced apart by about 90 degrees, about 120 degrees, about 135 degrees, about 180 degrees, or by another amount around the catheter shaft, such as, for example, through a wall of the catheter shaft around a central delivery lumen.

The steering element 403 is actuated by a knob 404. The knob 404 can include internal threads on an inner surface of the knob 404 that directly interact with outer threads of the steering element 403 to cause the steering element 403 to translate forward and back. In some implementations, the internal threads of the knob 404 interact with a separate component—e.g., a separate threaded member, tube, gear, or the like—that interacts with the steering element 403. For example, the knob 404 can be coupled to a gear or gear assembly that causes the steering element 403 to translate forward and back so that the knob 404 does not require any internal threads. The knob 404 can be configured such that the steering element 403 moves in a forward direction when the knob 404 is rotated in a clockwise direction and the steering element 403 moves in a backward direction when the knob 404 is rotated in a counterclockwise direction, or vice versa. Movement of the steering element 403 causes the one or more actuation elements 402 to move back and forth in a catheter shaft of the delivery system which can cause a distal region of the catheter to bend or straighten.

The steering system/mechanism 400 can also include one or more stops attached to the actuation element 402 such that movement of the steering element 403 does not cause the actuation element 402 to move until the steering element 403 engages one of the stops. Referring now to FIGS. 19 and 20, first and second stops 405, 406 are shown attached to the actuation element 402 on either side of the steering element 403. As the steering element 403 is moved in a first direction, the first stop 405 is engaged by a first end 407 of the steering element 403 so that a first portion 402A of the actuation element 402 is pushed by continued movement of the steering element 403 in the first direction and a second portion 402B of the actuation element 402 is pulled by continued movement of the steering element 403 in the first direction. As the steering element 403 is moved in a second direction, the second stop 406 is engaged by a second end 408 of the steering element 403 so that the first portion 402A of the actuation element 402 is pulled by continued movement of the steering element 403 in the second direction and the second portion 402B of the actuation element 402 is pushed by continued movement of the steering element 403 in the second direction.

In some implementations, the first and second stops 405, 406 can be arranged on the actuation element 402 so that the first and second stops 405, 406 abut the first and second ends 407, 408 of the steering element 403 at all times, thereby minimizing slack or backlash between the movement of the steering element 403 and movement of the actuation element 402. Optionally, the first and second stops 405, 406 can be spaced apart such that movement of the steering element 403 in the opposite direction does not immediately reverse the movement of the actuation element 402.

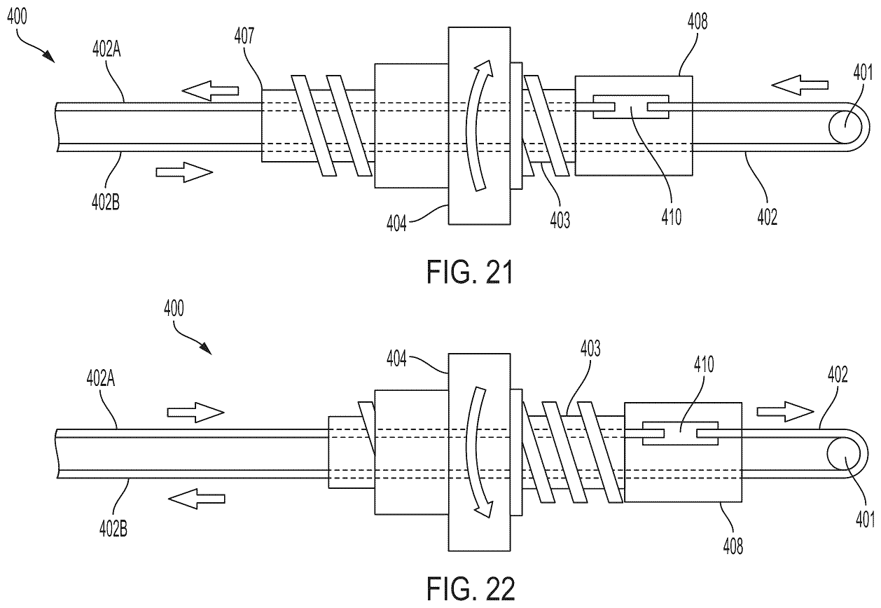

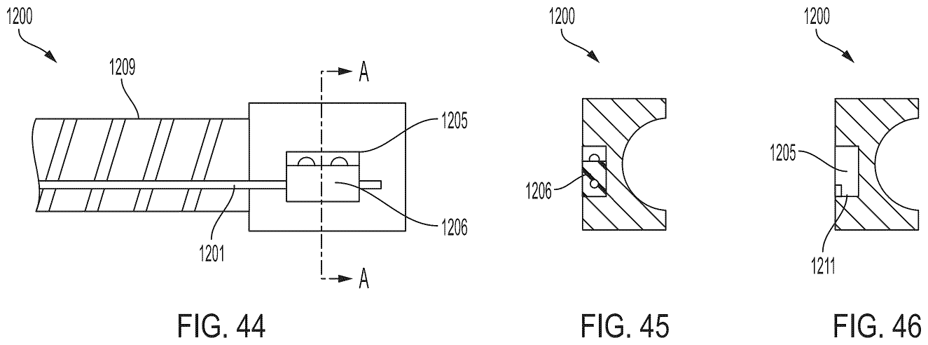

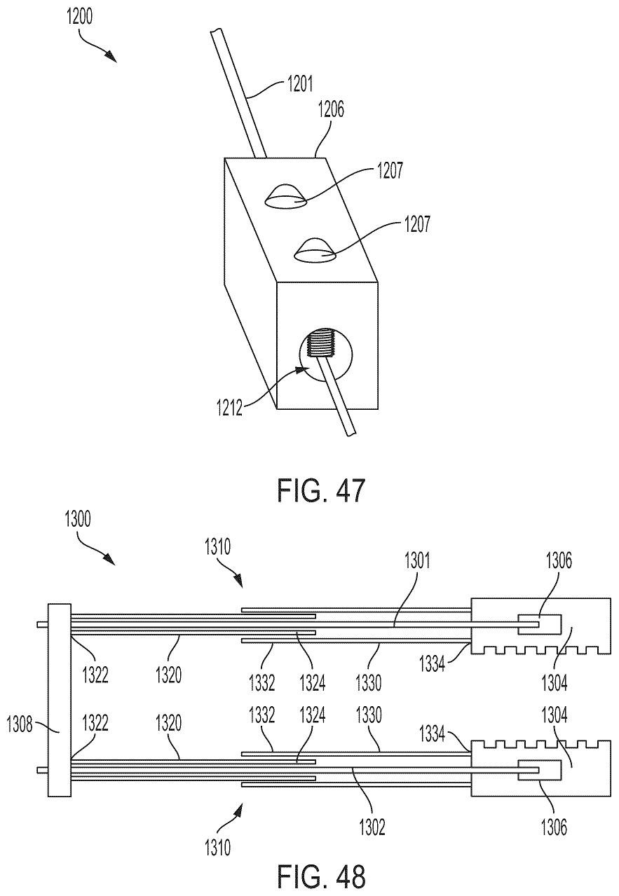

Referring now to FIGS. 21 and 22, the steering system/mechanism 400 can optionally include a grasping element 410 connected to the steering element 403. The grasping element 410 (e.g., clamp, connector, fastener, etc.) is connected to the ends of the first and second portions 402A, 402B of the actuation element 402. (An example of a clamp that can be used is shown in FIGS. 44-47.) As the steering element 403 is moved in the first direction the grasping element or clamp 410 engages the actuation element 402 so that the first portion 402A of the actuation element 402 is pushed by continued movement of the steering element 403 in the first direction and the second portion 402B of the actuation element 402 is pulled by continued movement of the steering element 403 in the first direction. As the steering element 403 is moved in the second direction the grasping element or clamp 410 engages the actuation element 402 so that the first portion 402A of the actuation element 402 is pulled by continued movement of the steering element 403 in the second direction and the second portion 402B of the actuation element 402 is pushed by continued movement of the steering element 403 in the second direction. Optionally, the first and second portions 402A, 402B of the actuation element 402 can be clamped by independent first and second clamps, respectively, that are each connected to the steering element 403.

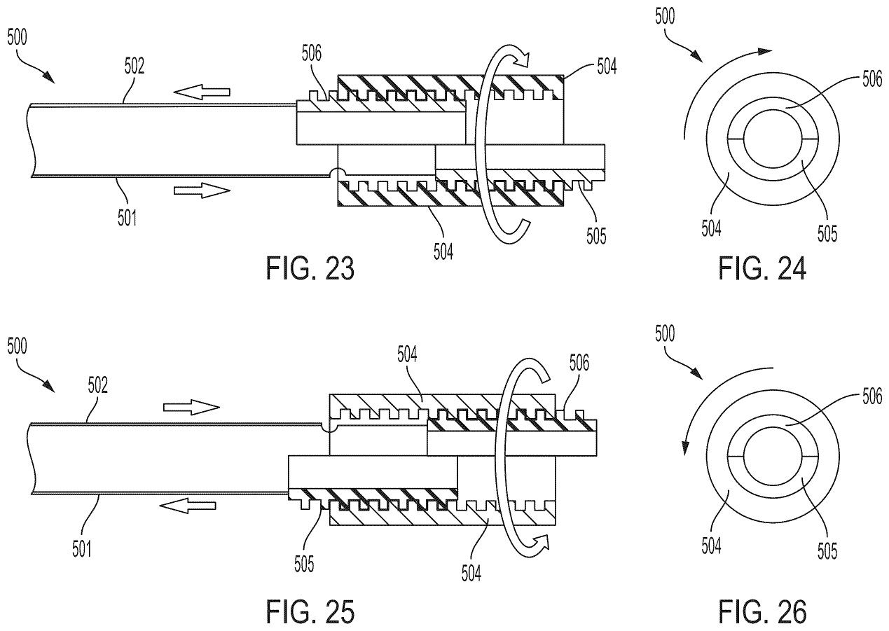

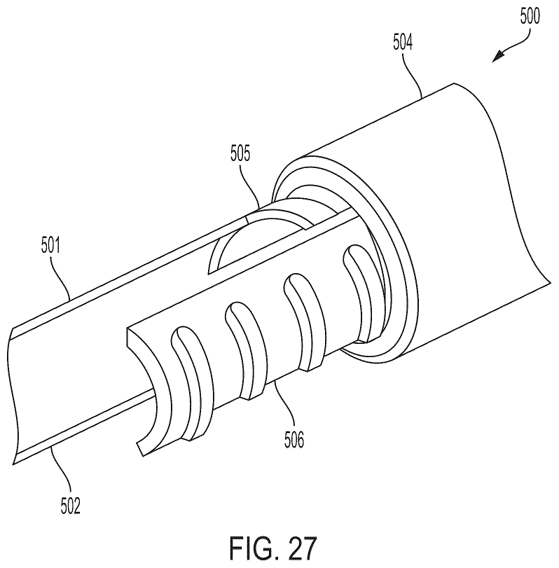

Referring now to FIGS. 23-27, an example steering system/mechanism 500 (e.g., assembly of components that work together to steer) for a delivery system is shown. The steering system/mechanism 500 can be included inside of a handle of the delivery system and can be attached to proximal end of a catheter shaft. In some implementations, the steering system/mechanism 500 can include a knob 504, a first actuation element 501 (e.g., control wires, pull wires, lines, sutures, wires, rods, etc.) connected to a first steering element 505 (e.g., threaded member, slider, follower, etc.), and a second actuation element 502 (e.g., control wires, pull wires, lines, sutures, wires, rods, etc.) connected to a second steering element 506 (e.g., threaded member, slider, follower, etc.). The first steering element 505 has a right-hand thread and the second steering element 506 has a left-hand thread. In some implementations, the knob 504 includes an internal double thread for engaging the threads of the first and second steering elements 505, 506.

Referring now to FIGS. 23-24, when the knob 504 is rotated in the clockwise direction the first steering element 505 pulls or exerts tension on a first actuation element 501 (also known as a steering wire) and the second steering element 506 releases tension and/or pushes a second actuation element 502. Referring now to FIGS. 25-26, when the knob 504 is rotated in the counter-clockwise direction the first steering element 505 releases tension on and/or pushes the first actuation element 501 and the second steering element 506 pulls and/or exerts tension on the second actuation element 502. The simultaneous pulling and releasing/pushing of the actuation elements 501, 502 provides bi-directional movement of distal end or distal end portion of the catheter. This movement is helpful particularly when the catheter has been held in a curved position for some time and has become set in the curved position, pushing and pulling on opposing actuation elements 501, 502 assists the operator in straightening the curved portion of the catheter shaft.

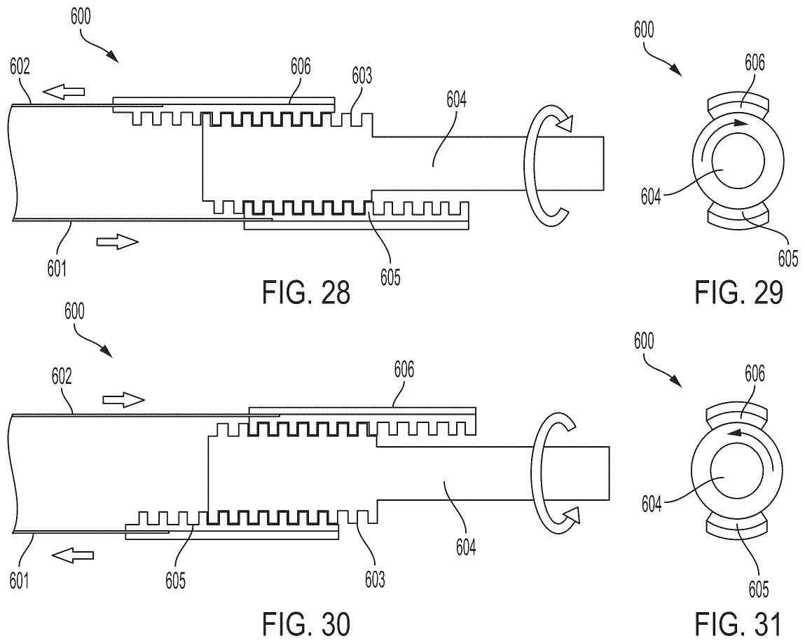

Referring now to FIGS. 28-33, an example steering system/mechanism 600 (e.g., assembly of components that work together to steer) for a delivery system is shown. The steering system/mechanism 600 is capable of simultaneously pulling and releasing actuation elements 601, 602 (e.g., control wires, pull wires, lines, sutures, wires, rods, etc.) and can be used with any delivery system disclosed herein. The steering system/mechanism 600 includes a double-threaded worm gear 604 and first and second racks 605, 606. A threaded portion 603 of the double-threaded worm gear 604 engages the first and second racks 605, 606 so that rotation of the worm gear causes the first and second racks 605, 606 to move proximally or distally. The first rack 605 has a right-hand thread and the second rack 606 has a left-hand thread. The first rack 605 is connected to the first actuation element 601 and the second rack 606 is connected to the second actuation element 602. The actuation elements 601, 602 are connected to the racks 605, 606 by any suitable means, such as with a grasping element disclosed herein.

In some implementations, when the double threaded worm gear 604 is rotated in the clockwise direction (FIGS. 28 and 29), the first rack 605 pulls the first actuation element 601 in a proximal direction and the second rack 606 moves in a distal direction to release the second actuation element 602. In some implementations, when the double threaded worm gear 604 is rotated in the counterclockwise direction (FIGS. 30 and 31), the first rack 605 moves in a distal direction to release the first actuation element 601 and the second rack 606 pulls the second actuation element 602 in a proximal direction. The simultaneous pulling and releasing of the actuation elements 601, 602 provides bi-directional movement of a distal end or distal end portion of a catheter to which the actuation elements 601, 602 are attached. The double threaded worm gear 604 can be rotated via a knob 608 arranged at a proximal end portion of the worm gear 604.

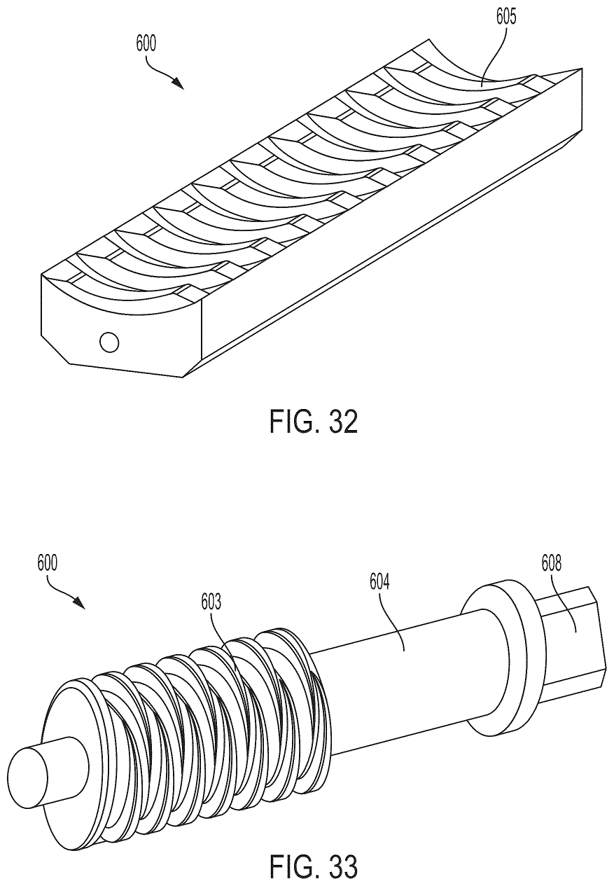

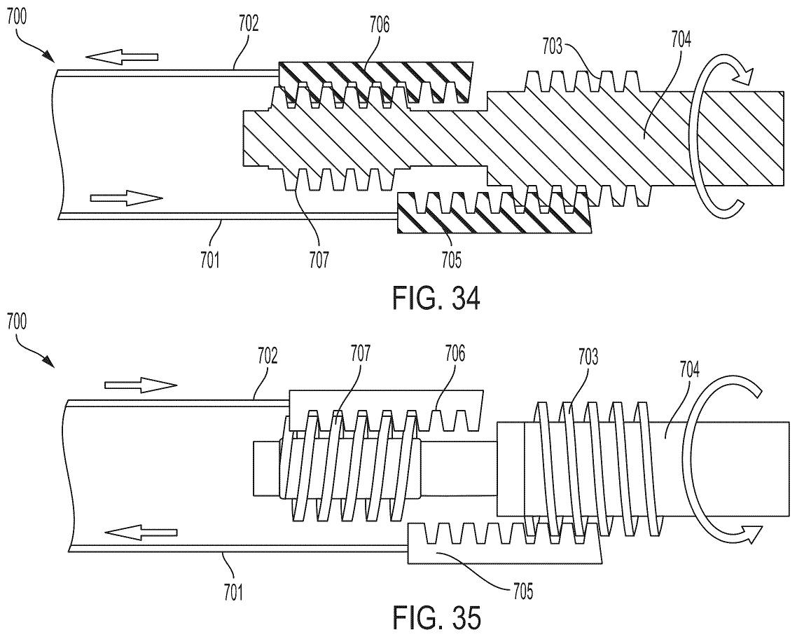

Referring now to FIGS. 34-35, an example steering system/mechanism 700 (e.g., assembly of components that work together to steer) for a delivery system is shown. The steering system/mechanism 700 is capable of simultaneously pulling and releasing actuation elements 701, 702 (e.g., control wires, pull wires, lines, sutures, wires, rods, etc.) and can be used with any delivery system disclosed herein. The steering system/mechanism 700 includes a bi-directional worm gear 704 having a proximal threaded portion 703 and a distal threaded portion 707. The threads of the proximal and distal threaded portions 703, 707 are opposite to each other, that is, the proximal threaded portion 703 has a right-hand thread when the distal threaded portion 707 has a left-hand thread, and vice versa. The diameter of the proximal and distal threaded portions 703, 707 can be the same or different. The proximal threaded portion 703 can have a greater diameter than the distal threaded portion 707, or vice versa. The steering system/mechanism 700 further includes a first rack 705 having threads that match those of the proximal threaded portion 703 and a second rack 706 having threads that match those of the distal threaded portion 707. The first rack 705 is connected to the first actuation element 701 and the second rack 706 is connected to the second actuation element 702. The actuation elements 701, 702 are connected to the racks 705, 706 by any suitable means, such as with a grasping element disclosed herein.

In some implementations, when the worm gear 704 is rotated in the clockwise direction (FIG. 34), the first rack 705 pulls the first actuation element 701 in a proximal direction and the second rack 706 moves in a distal direction to release the second actuation element 702. When the worm gear 704 is rotated in the counterclockwise direction (FIG. 35), the first rack 705 moves in a distal direction to release the first actuation element 701 and the second rack 706 pulls the second actuation element 702 in a proximal direction. The simultaneous pulling and releasing of the actuation elements 701, 702 provides bi-directional movement of a distal end or distal end portion of a catheter to which the actuation elements 701, 702 are attached.

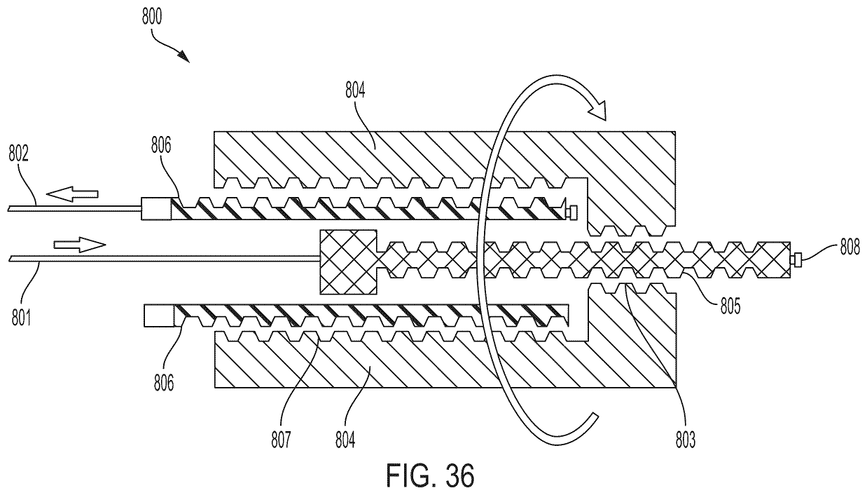

Referring now to FIG. 36, an example steering system/mechanism 800 (e.g., assembly of components that work together to steer) for a delivery system is shown. The steering system/mechanism 800 is capable of simultaneously pulling and releasing actuation elements 801, 802 (e.g., control wires, pull wires, lines, sutures, wires, rods, etc.) and can be used with any delivery system disclosed herein. The steering system/mechanism 800 includes a control member or knob 804 having internal threaded portions: a proximal threaded portion 803 and a distal threaded portion 807. The threads of the proximal and distal threaded portions 803, 807 are opposite to each other, that is, the proximal threaded portion 803 has a right-hand thread when the distal threaded portion 807 has a left-hand thread, and vice versa. The diameter of the proximal and distal threaded portions 803, 807 can be the same or different. The proximal threaded portion 803 can have a smaller diameter than the distal threaded portion 807, as shown in FIG. 36, or vice versa. The steering system/mechanism 800 further includes a first steering element 805 (e.g., threaded shaft, screw, etc.) having threads that match those of the proximal threaded portion 803 and a second steering element 806 (e.g., threaded shaft, screw, etc.) having threads that match those of the distal threaded portion 807. The first steering element 805 is connected to the first actuation element 801 and the second steering element 806 is connected to the second actuation element 802. The actuation elements 801, 802 are connected to the steering elements 805, 806 by any suitable means, such as with a grasping element (e.g., clamp, fastener, crimp, etc.) disclosed herein.

When the control member 804 is rotated in the clockwise direction (FIG. 34), the first steering element 805 pulls the first actuation element 801 in a proximal direction and the second steering element 806 moves in a distal direction to release the second actuation element 802. When the control member 804 is rotated in the counterclockwise direction, the first steering element 805 moves in a distal direction to release the first actuation element 801 and the second steering element 806 pulls the second actuation element 802 in a proximal direction. The simultaneous pulling and releasing of the actuation elements 801, 802 provides bi-directional movement of a distal end or distal end portion of a catheter to which the actuation elements 801, 802 are attached.

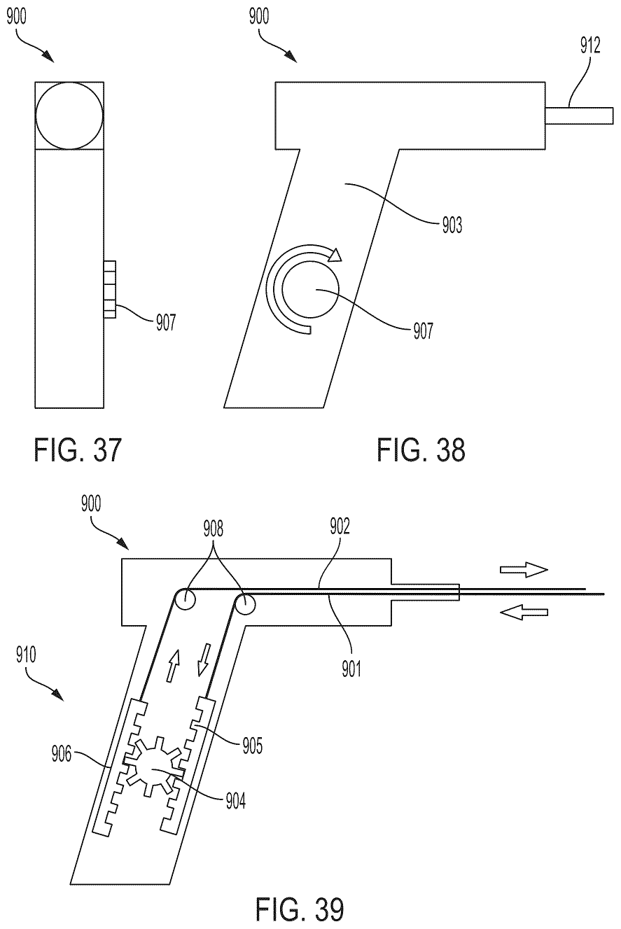

Referring now to FIGS. 37-39, an example control handle 900 including a steering system/mechanism 910 (e.g., assembly of components that work together to steer) for a delivery system is shown. The handle 900 is connected to a proximal end of a catheter shaft 912. A grip portion 903 of the control handle 900 is configured for an operator to grasp the control handle 900 and to operate a control member 907 (e.g., knob, handle, etc.) that actuates the steering system/mechanism 910. In some implementations, the steering system/mechanism 910 is capable of simultaneously pulling and releasing actuation elements 901, 902 (e.g., control wires, pull wires, lines, sutures, wires, rods, etc.) that extend through the catheter shaft 912.

In some implementations, the steering system/mechanism 910 includes a pinion gear 904 that engages toothed portions of first and second steering elements 905, 906 (e.g., gear rack, toothed member, etc.). The first steering element 905 is connected to the first actuation element 901 and the second steering element 906 (e.g., gear rack, toothed member, etc.) is connected to the second actuation element 902.

The actuation elements 901, 902 can be connected to the steering elements 905, 906 by any suitable means, such as with a grasping element disclosed herein. In some implementations, the actuation elements 901, 902 extend upward through the grip portion 903 of the control handle 900 and are redirected toward the catheter shaft 912 via pulleys 908. The actuation elements 901, 902 can be redirected by any suitable means, such as, for example, by eyelets, wheels, tubes, internal bearing surfaces handle, or the like.

In some implementations, when the control member 907 is rotated in the clockwise direction (FIG. 38), the pinion gear 904 similarly rotates to cause the first steering element 905 to pull the first actuation element 901 in a proximal direction and the second steering element 906 to move in a distal direction to release the second actuation element 902. In some implementations, when the control member 907 and pinion gear 904 are rotated in the counterclockwise direction, the first steering element 905 moves in a distal direction to release the first actuation element 901 and the second steering element 906 pulls the second actuation element 902 in a proximal direction. The simultaneous pulling and releasing of the actuation elements 901, 902 provides bi-directional movement of a distal end or distal end portion of the catheter shaft 912 to which the actuation elements 901, 902 (e.g., control wires, pull wires, lines, sutures, wires, rods, etc.) are attached.

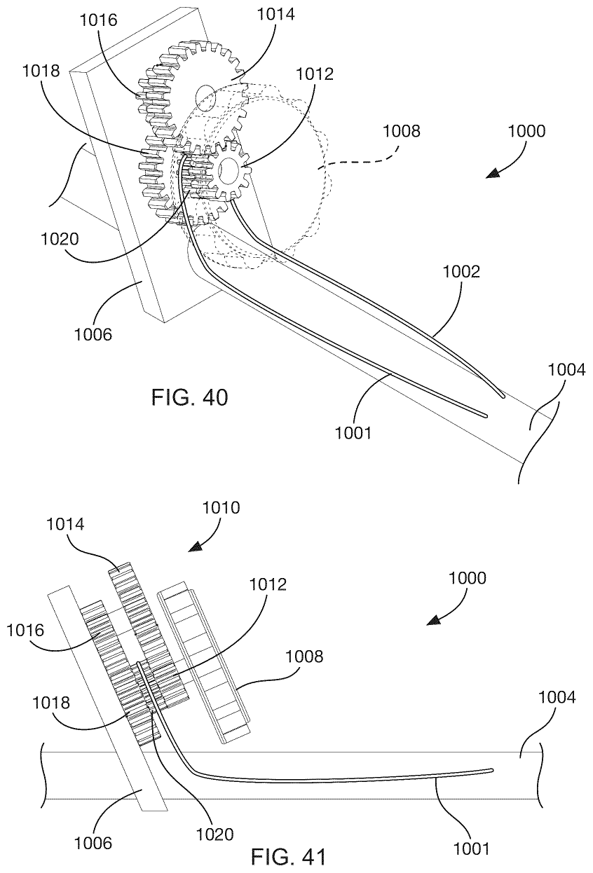

Referring now to FIGS. 40-41, an example steering system/mechanism 1000 (e.g., assembly of components that work together to steer) for a delivery system is shown. The steering system/mechanism 1000 is connected to a proximal end of a catheter shaft 1004 and includes a transmission 1010 for transmitting rotational movement of a control member 1008 (e.g., knob, handle, etc.) to the simultaneously pulling and releasing of actuation elements 1001, 1002 that extend through the catheter shaft 1004. The transmission 1010 is supported by a base 1006 that extends above the catheter shaft 1004 at an angle to provide access to the knob 1008. The steering system/mechanism 1000 can be housed within a handle (not shown).

In some implementations, the transmission 1010 includes multiple gears (e.g., 2, 3, 4, 5, 6, 7, or more gears). In some implementations, the transmission 1010 includes five gears: a first gear 1012, a second gear 1014, a third gear 1016, and a fourth gear 1018, and a fifth gear 1020. The knob 1008, first gear 1012, fourth gear 1018, and fifth gear 1020 are coaxially arranged. The knob 1008 and first gear 1012 have a fixed relationship such that the first gear 1012 rotates when the knob 1008 is rotated. Similarly, the second gear 1014 and third gear 1016 are coaxially arranged have a fixed relationship such that the second gear 1014 and third gear 1016 rotate together. The fourth gear 1018 and fifth gear 1020 also have a fixed relationship and rotate together.

In some implementations, during operation of the steering system/mechanism 1000, rotation of the knob 1008 at a first speed causes the first gear 1012 to turn at the first speed. Because the second gear 1014 has a larger diameter than the first gear 1012, rotating the first gear 1012 at the first speed causes the second gear 1014 to rotate at a second speed that is slower than the first speed. The third gear 1016 also rotates at the second speed because the third gear 1016 rotates together with the second gear 1014. Rotating the third gear 1016 at the second speed causes the fourth gear 1018 to rotate a third speed that is slower than the second speed because of the larger diameter of the fourth gear 1018 relative to the third gear 1016. The fifth gear 1020 also rotates at the third speed. Thus, the transmission 1010 reduces the rate of rotation from the first speed down to the third speed. This reduction in speed corresponds with a proportional increase in the torque output of the transmission 1010. That is, a smaller torque applied to the knob results in an amplified torque at the fifth gear 1020 by virtue of the mechanical advantage provided by the transmission 1010.

In some implementations, the actuation elements 1001, 1002 can be formed into a single loop that wraps around the fifth gear 1020. The fifth gear 1020 can be formed out of a high friction material, such as rubber, to engage the actuation elements 1001, 1002. In some implementations, an additional toothed belt (not shown) can also be provided that wraps around the fifth gear 1020 to transmit the torque of the knob 1008 through the transmission 1010 and to the actuation elements 1001, 1002. In some implementations, the actuation elements 1001, 1002 can be connected to the toothed belt by any suitable means, such as, with a grasping element disclosed herein. In some implementations, as the actuation elements 1001, 1002 extend from the transmission 1010 to the catheter shaft 1004 the actuation elements 1001, 1002 can be redirected by any suitable means, such as, for example, by eyelets, wheels, tubes, internal bearing surfaces handle, or the like.

In some implementations, when the control member 1008 is rotated in the clockwise direction the fifth gear 1020 similarly rotates in the clockwise direction to pull the first actuation element 1001 in a proximal direction and to release the second actuation element 1002. In some implementations, when the control member 1008 is rotated in the counter-clockwise direction, the fifth gear 1020 rotates in the counter-clockwise direction to release the first actuation element 1001 and to pull the second actuation element 1002 in a proximal direction. The simultaneous pulling and releasing of the actuation elements 1001, 1002 provides bi-directional movement of a distal end or distal end portion of the catheter shaft 1004 to which the actuation elements 1001, 1002 are attached.

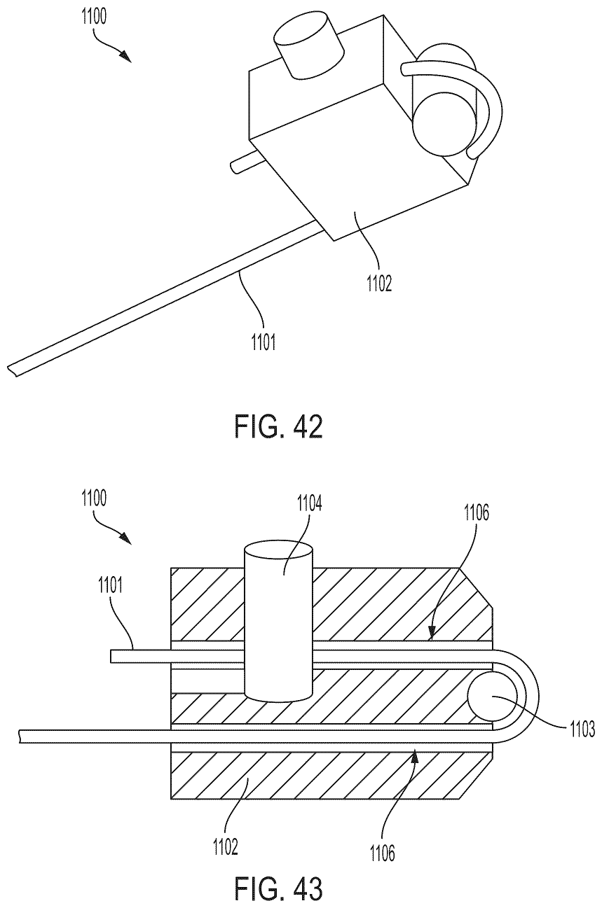

Referring now to FIGS. 42-43, a proximal end of an actuation element 1101 (e.g., control wires, pull wires, lines, sutures, wires, rods, etc.) is shown retained by an example grasping element 1102 (e.g., clamp, fastener, crimp, etc.) as part of a delivery system 1100. The actuation element 1101 is a round actuation element 1101. In some implementations, the actuation element 1101 is inserted into one of two slots or openings 1106 of the grasping element 1102, is bent around a bearing member 1103 (e.g. wheel, roller, ball bearing, roller bearing, etc.), and is inserted into the other of the two slots or openings 1106 where the end of the actuation element 1101 is retained in position by a set screw 1104. Wrapping the actuation element 1101 around the bearing member 1103 distributes the load across the surface of the bearing member 1103 to reduce the load experienced by the actuation element 1101 at any given point. The bearing member 1103 can be a pin, screw, rivet, or the like.

The grasping element 1102 can be attached to any suitable portion of the delivery system 1100, such as, for example, a clamp or a steering element of a steering system/mechanism. Optionally, the grasping element 1102 can be integrally formed with any suitable component of the delivery system 1100, such as, for example, the handles and steering systems/mechanisms described herein. The example grasping element 1102 enables the length of the actuation element 1101 to be adjusted by loosening the set screw 1104, repositioning the actuation element 1101, and tightening the set screw 1104 against the actuation element 1101 again. Thus, once the delivery system 1100 is assembled the length and/or tension of the actuation element 1101 can be adjusted.

Referring now to FIGS. 44-47, a proximal end of an actuation element 1201 (e.g., control wires, pull wires, lines, sutures, wires, rods, etc.) is shown retained by an example grasping element 1206 (e.g., clamp(s), set screw, etc.) as part of a delivery system 1200. The actuation element 1201 is a round actuation element 1201 that is inserted into a slot or opening 1212 of the grasping element 1206 and is retained in position by two set screws 1207. Optionally, the grasping element 1206 can be integrally formed with any suitable component of the delivery system 1200, such as, for example, the handles and steering systems/mechanisms described herein.

In some implementations, the grasping element 1206 is retained in a pocket 1205 of a steering element 1209 of a steering system/mechanism. In some implementations, the pocket 1205 can include an undercut 1211 or other retaining feature such that the grasping element 1206 is held in place and encourages to be retained within the pocket 1205 when the grasping element 1206 is subjected to a tensile load via the actuation element 1201. In some implementations, the actuation element 1201 can be adjusted within the grasping element 1206 by removing the grasping element 1206 from the pocket 1205 of the steering element 1209, loosening the screws 1207, repositioning the actuation element 1201, and retightening the screws 1207. In some implementations, the grasping element 1206 can then be inserted back into the pocket 1205 of the steering element 1209.

Referring now to FIG. 48, an example delivery system 1300 is shown. The delivery system 1300 includes actuation elements 1301, 1302 (e.g., control wires, pull wires, lines, sutures, wires, rods, etc.) that extend from steering elements 1304 (e.g. gear rack, threaded member, toothed member, etc.) of a steering system/mechanism (not shown) to a distal portion 1308 such as, for example, a pull ring, a low-profile pull ring, a hypotube, or the like. The actuation elements 1301, 1302 are attached to the steering elements 1304 by grasping elements 1306 (e.g., clamp, fastener, set screw, crimp, etc.) that can take on any suitable form, such as the grasping elements 1306 described herein.

In some implementations, to protect the actuation elements 1301, 1302 and reduce the likelihood of undesirable bending or kinking, a pair of example support structures 1310 surround the actuation elements 1301, 1302 and extend from the steering elements 1304 to the distal portion 1308. In some implementations, the support structures 1310 can be used to support actuation elements of any shape or thickness and of any of the delivery systems disclosed herein over longer unsupported distances to prohibit the actuation elements 1301, 1302 from being damaged from prolapsing, kinking, or the like. In some implementations, the support structures 1310 can be entirely contained within a handle of the delivery system 1300 such that the support structures 1310 do not extend into a catheter shaft. That is, the catheter shaft can serve the purpose of the support structures along the length of the catheter shaft while more space inside the handle may require additional support from the support structures.

In some implementations, the support structures 1310 include a first tube 1320 and a second tube 1330. In some implementations, a distal end 1322 of the first tube 1320 is connected to the distal portion 1308 and a proximal end 1324 of the first tube 1320 overlaps with the second tube 1330. In some implementations, a distal end 1332 of the second tube 1330 overlaps with the first tube 1320 and a proximal end 1334 of the second tube 1330 is connected to the steering elements 1304. Where the first tube 1320 has a smaller diameter than the second tube 1330, the proximal end 1324 of the first tube 1320 extends inside the second tube 1330, or vice versa. Because of the connection of the distal end 1322 of the first tube 1320 to the distal portion 1308 and the connection of the proximal end 1334 of the second tube 1330 to the steering elements 1304 the first and second tubes 1320, 1330 slide relative to each other as the actuation elements 1301, 1302 bend and flex in response to bending and flexing of the catheter or catheter shaft of the delivery system 1300. In other words, the support structures 1310 can be telescoping support structures that can change in length to accommodate changes in the length of the actuation elements 1301, 1302.

In some implementations, the inner diameter of the smaller of the first and second tubes 1320, 1330 can be about 5 percent to about 10 percent greater than the outer diameter of round actuation elements. Where the actuation elements are flat, the inner diameter of the smaller of the first and second tubes 1320, 1330 can be at least the width of the flat actuation elements, such as about 5 percent to about 10 percent greater than the width of the flat actuation elements, or about the same as the width of the flat actuation elements. The support structures 1310 can be formed from any suitable material, such as, for example, a plastic such as PTFE or a metal such as nitinol.

Referring now to FIG. 49, an example steerable catheter 1400 is shown that includes two catheter shafts: an outer shaft 1402 and an inner shaft 1404. The outer shaft 1402 is configured to reach a location above a center of an annulus, e.g., a mitral valve and a tricuspid valve annulus. In some implementations, the outer shaft 1402 has at least two sequentially arranged bending sections (see, e.g., FIGS. 8-13) so that the outer shaft 1402 can be bent and flexed so that a distal end 1406 of the outer shaft 1402 is arranged above and facing the native valve of a patient during an operation. In some implementations, once the position of the outer shaft 1402 has been fine-tuned, the inner shaft 1404 can be extended, bent, and rotated to place a distal end 1408 of the inner shaft 1404 at a desired location closer to the tissue of the valve.

In some implementations, the inner shaft 1404 can be controlled to extend distally so that the inner shaft 1404 becomes longer than the outer shaft 1402. Once extended to a desired length, the inner shaft 1404 can be manipulated by a steering system/mechanism to bend in a lateral direction 1410 and to twist or rotate in an axial direction 1412. These motions can be combined to move the extended and bent inner shaft 1404 in a sweeping motion 1405. Thus, rather than manipulating the distal end 1408 of the inner shaft 1404 via bending in two planes (similar to a cartesian coordinate system) the distal end 1408 of the inner shaft 1404 can be manipulated by bending and rotating (similar to a polar or spherical coordinate system). That is, the position of the distal end 1408 of the inner shaft 1404 can be specified by the extension length, the bend angle, and the rotation or twist angle of the inner shaft 1404. Thus, accessing a different location along the annulus of the native heart valve is a matter of rotating or twisting the inner shaft 1404 in the axial rotation direction 1412 a desired amount while maintaining the same bend angle and extension distance.

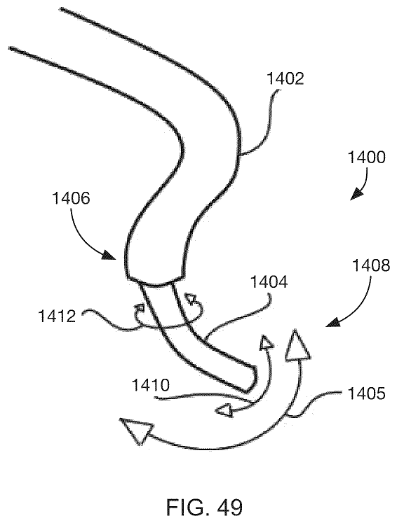

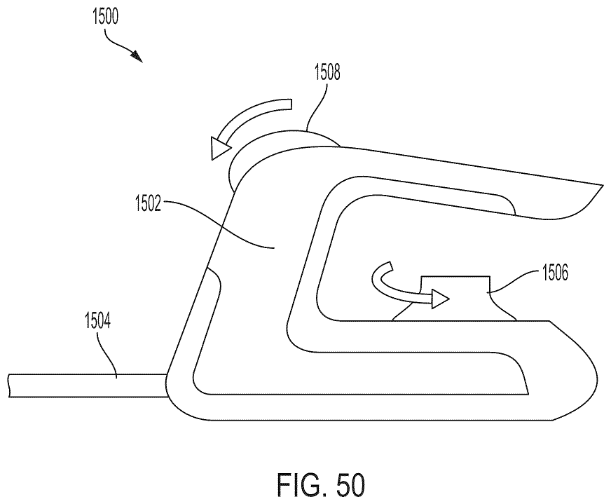

Referring now to FIG. 50, an example control handle 1502 for a delivery system 1500 is shown. In some implementations, the control handle 1502 is attached to a proximal end of a catheter shaft 1504 and includes first and second control members 1506, 1508 (e.g., knobs, buttons, slides, etc.) for actuating a steering system/mechanism (not shown) housed by the control handle 1502. In some implementations, the first and second control members 1506, 1508 actuate first and second steering systems that enable the control of first and second sets of actuation elements (not shown) that extend down the catheter shaft 1504 to manipulate a distal end or distal end portion of the delivery system 1500, respectively. In some implementations, the control handle 1502 is shaped somewhat like an iron but can take on a wide variety of shapes with varying arrangements of the first and second control members 1506, 1508. Control handles disclosed herein, such as the control handle 1502 of the delivery system 1500 can have a variety of configurations, shapes, and sizes depending on the type of steering system/mechanism and steering elements located inside the handle and depending on the use of the catheter.

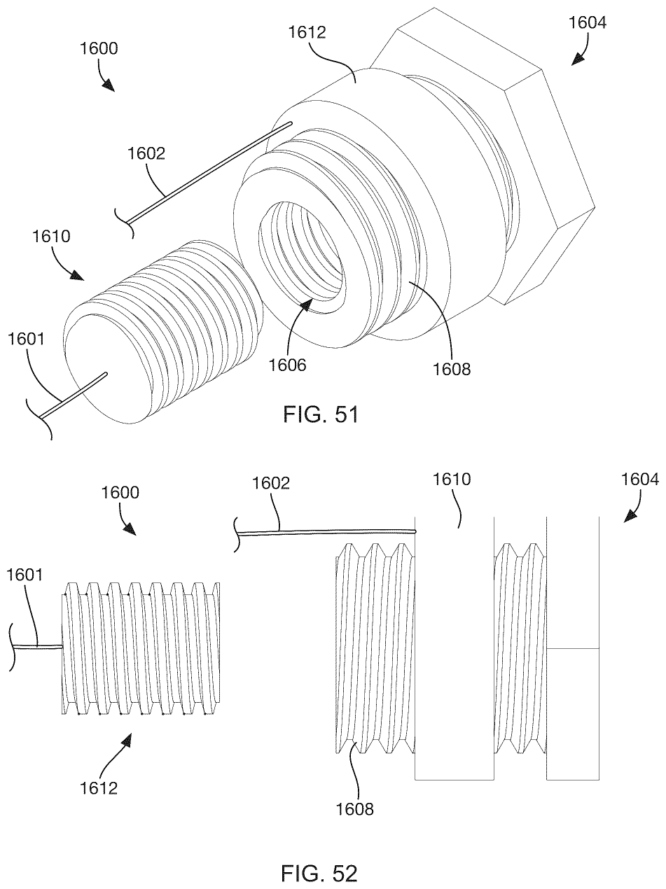

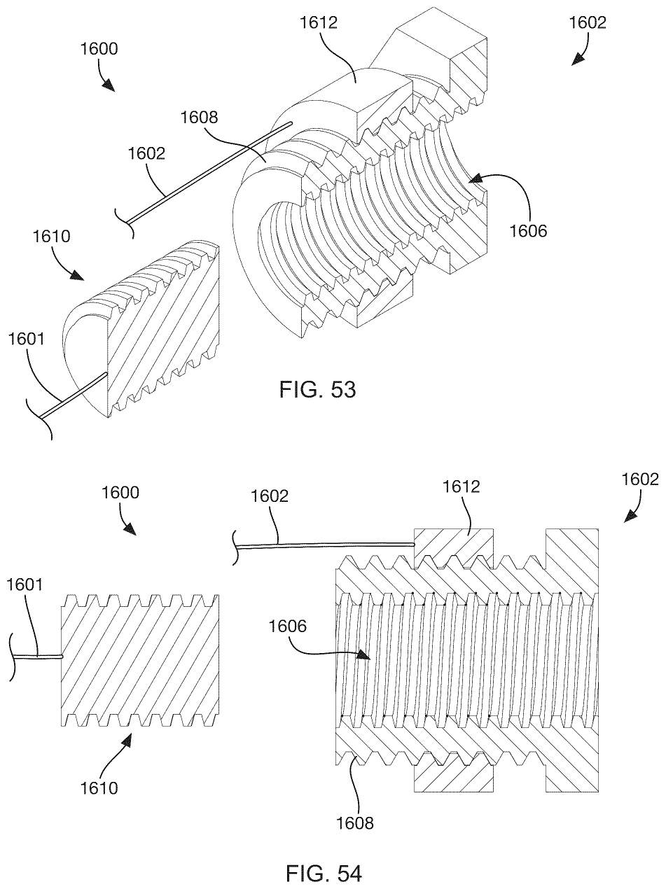

Referring now to FIGS. 51-54, an example steering system/mechanism 1600 (e.g., assembly of components that work together to steer) for a delivery system is shown. The steering system/mechanism 1600 is capable of simultaneously pulling and releasing actuation elements 1601, 1602 (e.g., control wires, pull wires, lines, sutures, wires, rods, etc.) and can be used with any delivery system disclosed herein.

In some implementations, the steering system/mechanism 1600 includes a control member 1604 having an internal threaded portion 1606 and an external threaded portion 1608. In some implementations, the threads of the internal and external threaded portions 1606, 1608 are opposite to each other, that is, the internal threaded portion 1606 has a right-hand thread when the external threaded portion 1608 has a left-hand thread, and vice versa.

In some implementations, the steering system/mechanism 1600 further includes a first steering element 1610 (e.g., threaded shaft, screw, bolt, etc.) having external threads that match those of the internal threaded portion 1606 and a second steering element 1612 (e.g., threaded shaft, nut, etc.) having internal threads that match those of the external threaded portion 1608. (The first steering element 1610 threads into the internal threaded portion 1606 of the knob 1604 but is shown disassembled from the knob 1604 for illustration purposes.) In some implementations, the first steering element 1610 is connected to the first actuation element 1601 and the second steering element 1612 is connected to the second actuation element 1602. The actuation elements 1601, 1602 are connected to the steering elements 1610, 1612 by any suitable means, such as welding or with a grasping element disclosed herein.

In some implementations, when the control member 1604 is rotated in the clockwise direction, the first steering element 1610 pulls the first actuation element 1601 in a proximal direction and the second steering element 1612 moves in a distal direction to release the second actuation element 1602. In some implementations, when the control member 1604 is rotated in the counterclockwise direction, the first steering element 1610 moves in a distal direction to release the first actuation element 1601 and the second steering element 1612 pulls the second actuation element 1602 in a proximal direction. The simultaneous pulling and releasing of the actuation elements 1601, 1602 provides bi-directional movement of a distal end and/or distal end portion of a catheter or catheter shaft to which the actuation elements 1601, 1602 are attached.

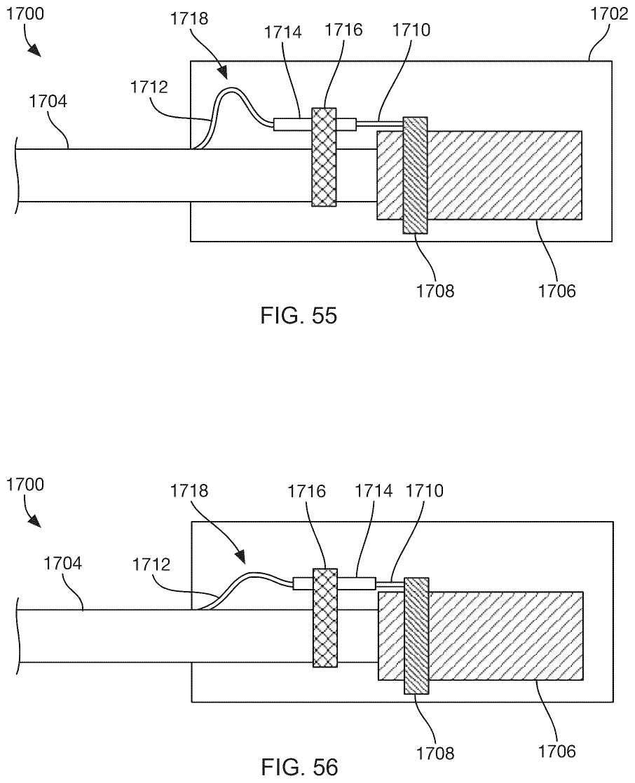

Referring now to FIGS. 55 and 56, an example delivery system 1700 is shown. The delivery system 1700 includes a handle 1702, a catheter shaft 1704 extending from the handle 1702, a steering system/mechanism 1706 (e.g. assembly of components that work together to steer) with a steering element 1708 (e.g. drive, puller, worm gear, winch, etc.) that is connected to an actuation element 1710 (e.g., control wires, pull wires, lines, sutures, wires, rods, etc.). The actuation element 1710 extends through a compression member 1712 (e.g., compression coils, compression springs, compression tubes, etc.) that extends distally from a stopper 1714, into the catheter shaft 1704, and to a distal end (not shown) of the catheter shaft 1704. In some implementations, the compression member 1712 is a compression coil and the stopper 1714 is a coil stopper. The compression member 1712 can attach to an end of the stopper 1714 or a side of the stopper 1714.

In some implementations, the compression member 1712 transmits or is configured to transmit compression forces from a distal end of the delivery system 1700 through the catheter shaft 1704 to the handle 1702 to reduce the impact of the compression forces on the performance of the delivery system 1700. The compression forces can be a result of, for example, the retraction of the actuation element 1710 to bend or flex the distal end of the delivery system 1700. The length of the compression member 1712 is tailored to the length of the catheter shaft 1704. A compression member 1712 that is too long can cause friction within the catheter shaft 1704 that makes it difficult to operate the delivery system 1700. A compression member 1712 that is too short can damage the delivery system 1700 and can make it harder to bend or flex the catheter shaft 1704 and otherwise operate the delivery system 1700.

In some implementations, a service loop 1718 formed between the stopper 1714 and the catheter shaft 1704 provides some additional material or slack in the compression member 1712 to accommodate relatively small changes in the length of the compression member 1712 as the catheter shaft 1704 is bent or flexed. In some implementations, the service loop 1718 is formed during initial assembly of the delivery system 1700 wherein the compression member 1712 is installed along the catheter shaft 1704, measured to the appropriate length, marked, and trimmed by the operator. The trimming operation adds time to the procedure and can be a difficult task when the compression member 1712 is formed from a tough or hard material.

A trimming step during assembly of the delivery is not required by the example delivery system 1700 shown in FIGS. 55 and 56. That is, the stopper 1714 at the proximal end of the compression member 1712 of the delivery system 1700 is adjustably connected to the catheter shaft 1704 by a mounting portion 1716 so that the length of the compression member 1712 can be adjusted after the delivery system 1700 is assembled. Consequently, the compression member 1712 can be cut to a desired length during manufacturing so that no trimming of the compression member 1712 is required by the operator. Minor adjustments to the length of the compression member 1712 can then be made by adjusting the position of the stopper 1714. The position of the stopper 1714 relative to the mounting portion 1716 can be adjusted in a wide variety of ways. For example, the stopper 1714 can be formed from threaded body that is threaded into a threaded opening of the mounting portion 1716 so that the stopper 1714 is translated proximally or distally by rotating the stopper 1714. The stopper 1714 can also be retained within an opening of the mounting portion 1716 by set screws, a clamp, or the like.

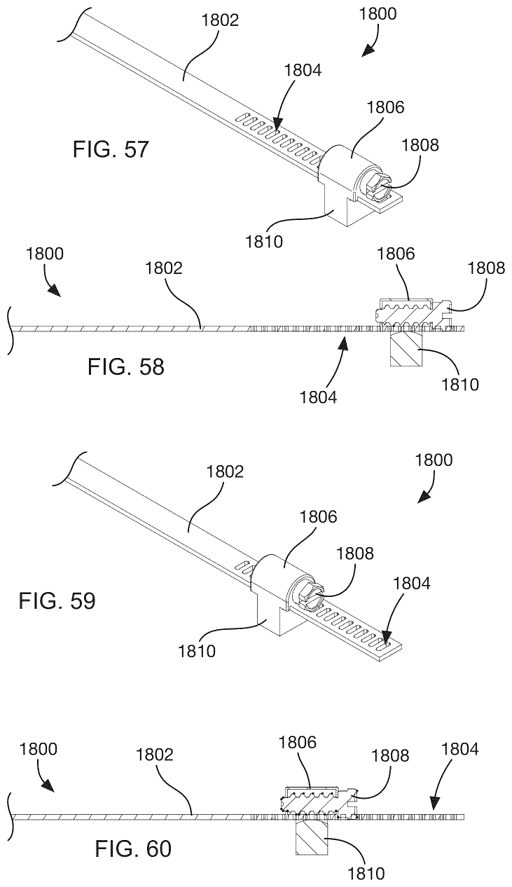

Referring now to FIGS. 57-60, a proximal end of an actuation element 1802 (e.g., control wires, pull wires, lines, sutures, wires, rods, etc.) is shown retained by an example grasping element 1806 (e.g., clamp, worm drive housing, etc.) as part of a delivery system 1800. In the illustrated implementation, the actuation element 1802 is a flat actuation element 1802 with a rectangular cross-sectional shape that includes a plurality of evenly spaced apart slots or holes 1804 arranged at the proximal end of the actuation element 1802. In some implementations, the actuation element 1802 is inserted into a slot or opening of the grasping element 1806 such that the spaced apart slots 1804 are engaged by threads of an adjustment screw 1808 retained within the grasping element 1806.

In some implementations, the grasping element 1806 further includes a base 1810 that can be attached to any suitable portion of the delivery system 1800, such as, for example, a clamp or a steering element of a steering system/mechanism. Optionally, the base 1810 can be integrally formed with any suitable component of the delivery system 1800, such as, for example, the handles and steering systems/mechanisms described herein.

In some implementations, the example grasping element 1806 enables the length of the actuation element 1802 to be adjusted via the adjustment screw 1808. Turning the adjustment screw 1808 causes the actuation element 1802 to retract into or extend from the grasping element 1806. Thus, once the delivery system 1800 is assembled the length and/or tension of the actuation element 1802 can be adjusted by tightening or loosening the adjustment screw 1808. In some implementations, the grasping element 1806 can further include a lock washer or cap to prohibit movement of the adjustment screw 1808 after the desired adjustment to the actuation element 1802 has been made.

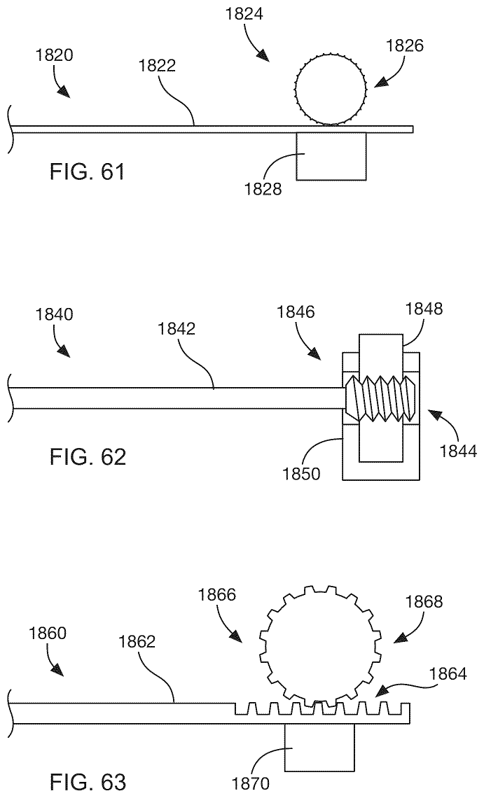

Referring now to FIGS. 61-63, some example adjustable grasping elements 1824, 1846, 1866 (e.g., clamps, drive gear(s), etc.) with actuation elements 1822, 1842, 1862 (e.g., control wires, pull wires, lines, sutures, wires, rods, etc.) for delivery systems 1820, 1840, 1860, respectively. Referring now to FIG. 61, the delivery system 1820 includes the actuation element 1822 that is a flat actuation element 1822 that extends through the grasping element 1824. In some implementations, the grasping element 1824 includes a textured wheel 1826 that presses the actuation element 1822 against a base 1828. In some implementations, the textured wheel 1826 engages the surface of the flat actuation element 1822 that can include slots or holes like the actuation element 1802, above. Friction between the textured wheel 1826 and the surface of the actuation element 1822 enables the position of the actuation element 1822 to be adjusted relative to the grasping element 1824, thereby changing the length and/or tension of the actuation element 1822.

Referring now to FIG. 62, the delivery system 1840 includes the actuation element 1842 that is a round actuation element 1842 that includes a threaded end portion 1844 that extends through the grasping element 1846. In some implementations, the threaded end portion 1844 is engaged by a threaded collar 1848 that is rotatably retained by or otherwise connected to a base 1850. In some implementations, rotating the threaded collar 1848 enables the position of the actuation element 1842 to be adjusted relative to the grasping element 1846, thereby changing the length and/or tension of the actuation element 1842.

Referring now to FIG. 63, the delivery system 1860 includes the actuation element 1862 that is a round actuation element 1862 that includes a plurality of slots 1864, the actuation element 1862 extending through the grasping element 1866. In some implementations, the slots 1864 are formed in one side of the actuation element 1862 or extend around the entire circumference of the actuation element 1862. In some implementations, the slots 1864 are engaged by a toothed wheel or gear 1868 that presses the actuation element 1862 against a base 1870. In some implementations, the engagement of slots 1864 by the toothed wheel 1868 enables the position of the actuation element 1862 to be adjusted relative to the grasping element 1866, thereby changing the length and/or tension of the actuation element 1862.

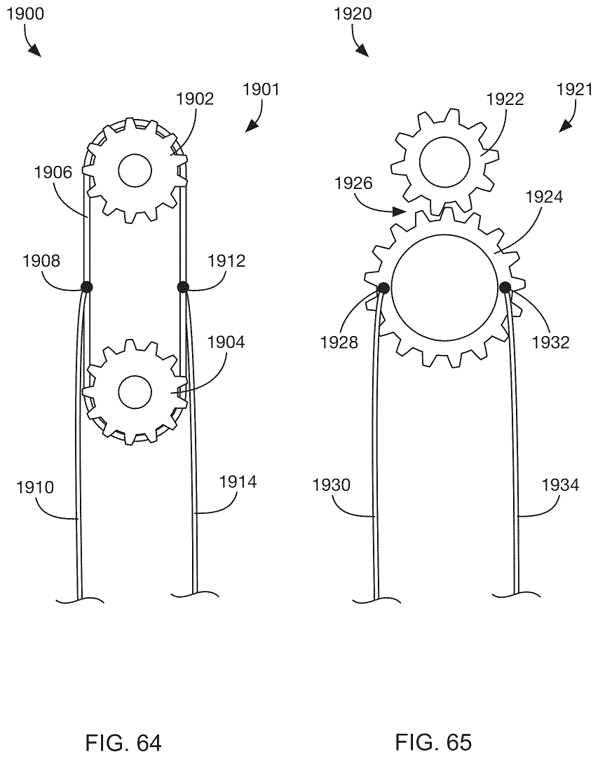

Referring now to FIG. 64, a steering system/mechanism 1901 (e.g., assembly of components that work together to steer) for an example delivery system 1900 is shown. In some implementations, the steering system/mechanism 1901 includes a drive gear 1902, a driven gear 1904, and a drive member 1906 (e.g., belt, chain, etc.). In some implementations, the drive member 1906 can be a chain that engages teeth of the drive and/or driven gears 1902, 1904 or, optionally, a belt that engages the drive and/or driven gears 1902, 1904 that do not include teeth.

In some implementations, a first attachment member 1908 (e.g. connector, coupler, fastener, clamp, etc.) connects a first actuation element 1910 (e.g., control wires, pull wires, lines, sutures, wires, rods, etc.) to one side of the drive member 1906 and a second attachment member connects a second actuation element 1914 (e.g., control wires, pull wires, lines, sutures, wires, rods, etc.) to the other side of the drive member 1906.

In some implementations, during operation, rotating the drive gear 1902 in a clockwise direction exerts a tension force on the first actuation element 1910 while releasing tension on the second actuation element 1914 and rotating the drive gear 1902 in a counterclockwise direction exerts a tension force on the second actuation element 1914 while releasing tension on the first actuation element 1910. Thus, the steering system/mechanism 1901 can be used to actuate a distal end portion of a delivery system to cause the distal end portion to bend or flex. In some implementations, an additional backspin prevention mechanism (not shown) can also be included to inhibit or prohibit the tension on the actuation elements 1912, 1914 from rotating the drive and/or driven gears 1902, 1904.

Referring now to FIG. 65, a steering system/mechanism 1921 (e.g., assembly of components that work together to steer) for an example delivery system 1920 is shown. In some implementations, the steering system/mechanism 1921 includes a drive gear 1922 and a driven gear 1924 that mesh together at an engagement point 1926. In some implementations, a first attachment member 1928 connects a first actuation element 1930 (e.g., control wires, pull wires, lines, sutures, wires, rods, etc.) to one side of the driven gear 1924 and a second attachment member 1932 (e.g., control wires, pull wires, lines, sutures, wires, rods, etc.) connects a second actuation element 1934 to the other side of the driven gear 1924.

In some implementations, during operation, rotating the drive gear 1922 in a counterclockwise direction exerts a tension force on the first actuation element 1930 while releasing tension on the second actuation element 1934 and rotating the drive gear 1922 in a clockwise direction exerts a tension force on the second actuation element 1934 while releasing tension on the first actuation element 1930. Thus, the steering system/mechanism 1921 can be used to actuate a distal end portion of a delivery system to cause the distal end portion to bend or flex. In some implementations, an additional backspin prevention mechanism (not shown) can also be included to prohibit or inhibit the tension on the actuation elements 1930, 1934 from rotating the drive and/or driven gears 1922, 1924.

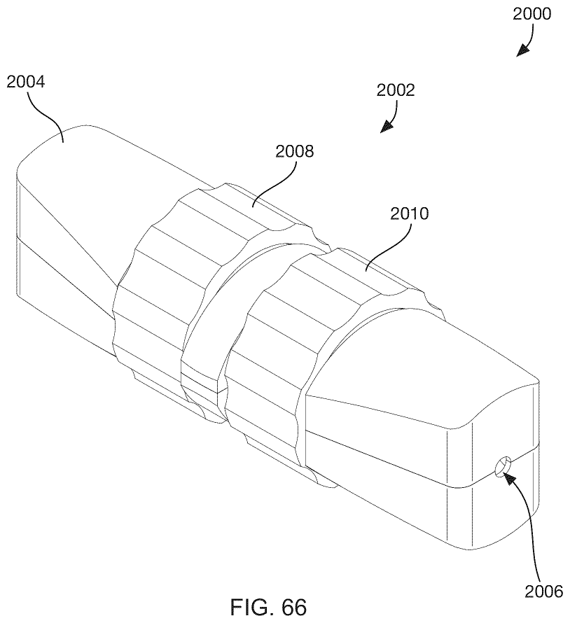

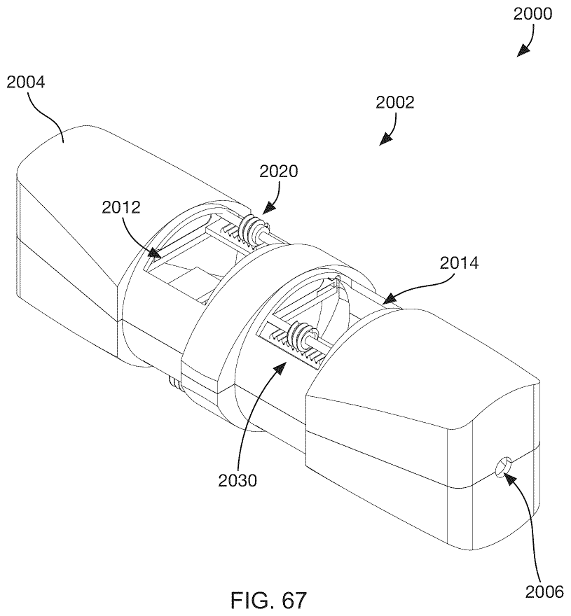

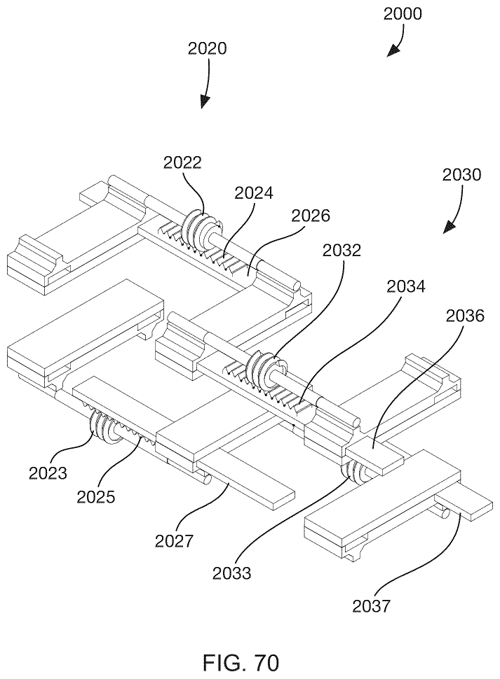

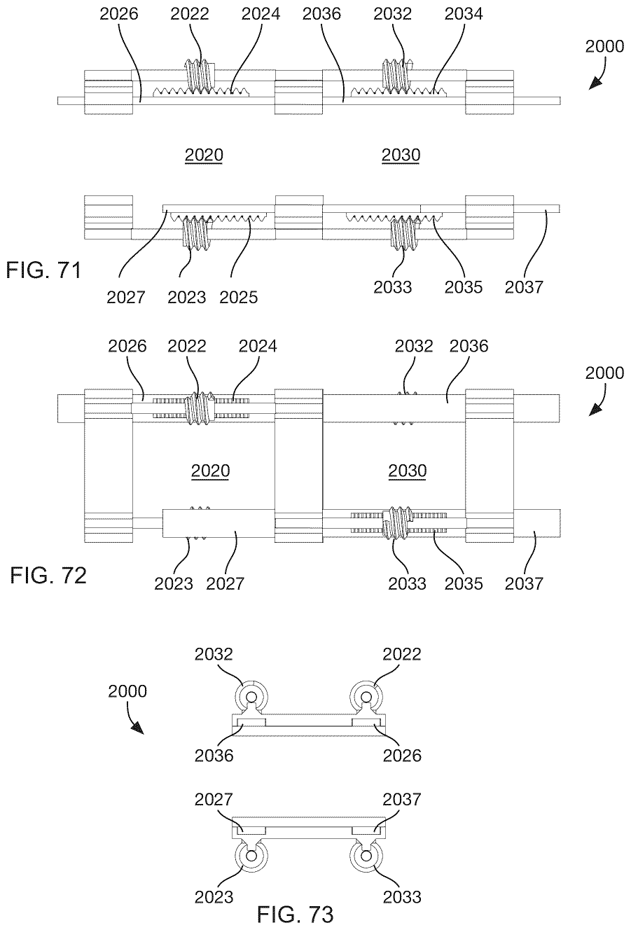

Referring now to FIGS. 66-73, a control handle 2002 including steering system/mechanisms 2020, 2030 (FIGS. 67-73) for an attached catheter or catheter shaft (not shown in FIGS. 66-73) of an example delivery system 2000 are shown. The control handle 2002 can be attached to a catheter, catheter shaft, or other elongated and steerable tubular or transluminal device for insertion into a patient. The control handle 2002 is an omni-directional control handle in that the control handle 2002 facilitates control of a distal end portion of a catheter shaft to be bent or flexed in any direction, that is, in a full 360-degree circle around the catheter shaft.

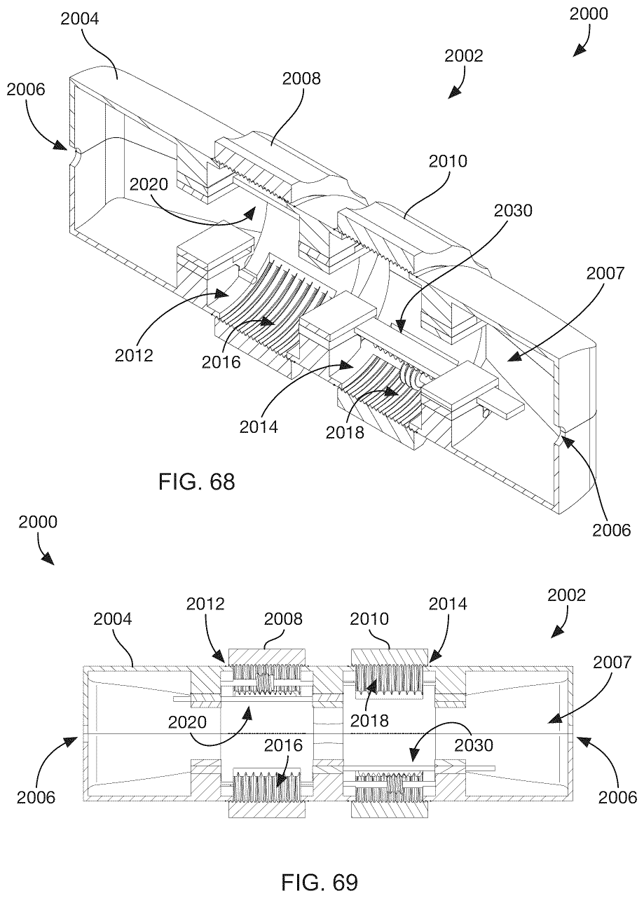

In some implementations, the control handle 2002 includes a housing 2004 that has openings 2006 on each end through which actuation elements (not shown) extend from the control handle 2002 to a catheter shaft (not shown). In some implementations, the openings 2006 form a luminal access 2007 (FIGS. 68 and 69) that extends longitudinally through the control handle 2002 for passage of other devices, actuation elements, and/or fluids through the handle 900 and the attached catheter.

In some implementations, the handle includes one or more control members, e.g., a first control member 2008 (e.g., a knob, button, switch, gear, etc.) and a second control member 2010 (e.g., a knob, button, switch, gear, etc.).

In some implementations, the first control member 2008 and the second control member 2010 are arranged concentrically with a longitudinal axis of the control handle 2002 and are rotatably attached to the housing 2004. In some implementations, the arrangement of the first and second control members 2008, 2010 on the exterior of the control handle 2002 provides the operator with increased leverage when actuating the control members 2008, 2010. That is, the diameter of the control members 2008, 2010 is greater and therefore provides more mechanical advantages than knobs that are disposed within a control handle.

In some implementations, actuating (e.g., rotating, etc.) the first and second control members 2008, 2010 actuates first and second steering systems/mechanisms 2020, 2030 (e.g., assembly of components that work together to steer), respectively, that are contained inside the control handle 2002. The first and second steering systems/mechanisms 2020, 2030 can be the same as or similar to other steering systems/mechanisms described herein.

In some implementations, the first control member 2008 covers first actuation openings 2012 that provide access to the first steering system/mechanism 2020. An interior thread 2016 of the first control member 2008 engages first and second drive gears 2022, 2023 of the first steering system/mechanism 2020 to cause the drive gears 2022, 2023 to rotate.

In some implementations, when rotated, the drive gears 2022, 2023 engage first and second racks 2024, 2025 that are connected to first and second steering elements 2026, 2027 (e.g., gear rack, toothed rack, threaded member, etc.) to cause the first and second steering elements 2026, 2027 to extend or retract. The first and second drive gears 2022, 2023 are threaded in opposite directions and engage opposite threaded portions of the interior thread 2016 of the knob 2008 so that rotation of the first control member 2008 causes the first drive gear 2022 to rotate opposite the second drive gear 2023. Consequently, the first and second steering elements 2026, 2027 move in opposite axial directions when the first control member 2008 is rotated. That is, the first steering element 2026 extends as the second steering element 2027 retracts, and vice versa. In some implementations, actuation elements attached to the steering elements 2026, 2027 that move proximally with respect to the housing 2004 increase in tension and actuation elements (e.g., control wires, pull wires, lines, sutures, wires, rods, etc.) attached to steering elements 2026, 2027 that move distally relax. In some implementations, the attached catheter or catheter shaft flexes in the direction of the tensed actuation elements. Thus, control of magnitude and direction of flex is not independent for control handle 2002.

In some implementations, the second control member 2010 covers second actuation openings 2014 that provide access to the second steering system/mechanism 2030. In some implementations, an interior thread 2018 of the second control member 2010 engages first and second drive gears 2032, 2033 of the second steering system/mechanism 2030 to cause the drive gears 2032, 2033 to rotate.

In some implementations, when rotated, the drive gears 2032, 2033 engage first and second racks 2034, 2035 that are connected to first and second steering elements 2036, 2037 to cause the first and second steering elements 2036, 2037 to extend or retract. In some implementations, the first and second drive gears 2032, 2033 are threaded in opposite directions and engage opposite threaded portions of the interior thread 2018 of the knob 2010 so that rotation of the second control member 2010 causes the first drive gear 2032 to rotate opposite the second drive gear 2033. Consequently, the first and second steering elements 2036, 2037 move in opposite axial directions when the second control member 2010 is rotated. That is, the first steering element 2036 extends as the second steering element 2037 retracts, and vice versa. In some implementations, actuation elements attached to the steering elements 2036, 2037 that move proximally with respect to the housing 2004 increase in tension and actuation elements attached to steering elements 2036, 2037 that move distally relax. The attached catheter or catheter shaft flexes in the direction of the tensed actuation elements. Thus, control of magnitude and direction of flex is not independent for control handle 2002.

In some implementations, the steering elements 2026, 2027, 2036, 2037 of the first and second steering systems/mechanisms 2020, 2030 can be connected to the proximal ends of actuation elements via grasping elements, such as those disclosed herein, or can be portions of the actuation elements themselves. For example, the actuation elements can be flat actuation elements that are welded or otherwise attached to the racks 2024, 2025, 2034, 2035 of the first and second steering systems/mechanisms 2020, 2030. While the racks 2024, 2025, 2034, 2035 are shown as toothed portions, the racks 2024, 2025, 2034, 2035 could also be formed from a plurality of slots that are engaged by the threads of the drive gears 2022, 2023, 2032, 2033 in the same fashion as the adjustable grasping element 1806 described above and shown in FIGS. 57-60.

In some implementations, the steering elements 2026, 2027, 2036, 2037 are arranged in opposing pairs: first and second steering elements 2026, 2027 of the first steering system/mechanism 2020; and first and second steering elements 2036, 2037 of the second steering system/mechanism 2030. The opposing pairs of steering elements 2026, 2027, 2036, 2037 are arranged 180 degrees from each other so that the first steering elements 2026, 2036 are opposite the second steering elements 2027, 2037. Thus, the first knob 2008 controls flexing of an attached catheter or catheter shaft in a first bending plane and the second knob 2010 controls flexing of an attached catheter or catheter shaft in a second bending plane that is orthogonal to the first bending plane. Combining bend magnitudes in each of the first and second bending planes enables the catheter or catheter shaft to be bent or flexed in any direction.

While the steering systems/mechanisms 2020, 2030 are shown with manually actuated knobs 2008, 2010, the steering systems/mechanisms 2020, 2030 can be actuated by other means, such as, for example, electrical motors or other actuators. Additionally, it should be noted that additional combinations of steering systems/mechanisms and knobs can be stacked longitudinally with the first and second steering systems/mechanisms to provide controls for additional mechanisms and/or to control bending in additional bending planes.

As noted above, a delivery system for a device or implant (e.g., an implantable prosthetic device, a prosthetic spacer device, a valve repair device, a valve replacement device, etc.) can include a distal end of a delivery sheath or means for delivery that can be flexed by the operator to align and position the implantable device within the opening of the native mitral valve. The flexible portion of the distal end of the delivery system can be incorporated into the delivery sheath or means for delivery and can be accomplished via the attachment of a flexible device. Various implementations of articulation devices—in particular, telescoping articulation devices—for attaching to a distal end of a delivery system or sheath are described herein. The telescoping articulation devices can be used with a variety of different catheters or sheaths. The catheters described above are one of the many different catheters that the telescoping articulation devices can be used with. The telescoping articulation devices can be used on catheters and sheaths that are steerable and catheters that are not steerable. Any steering system/mechanism can be employed when the catheter or sheath is steerable.

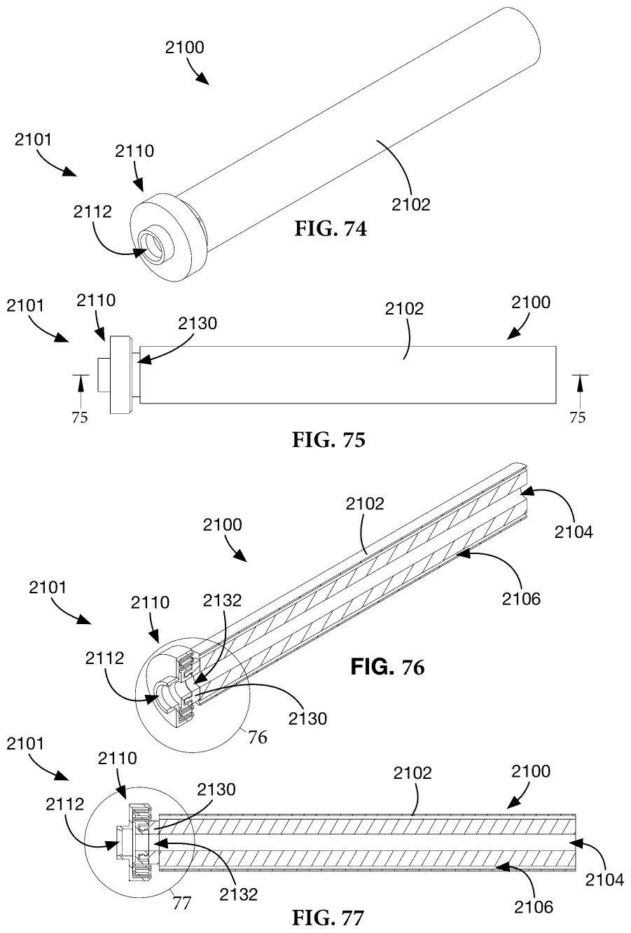

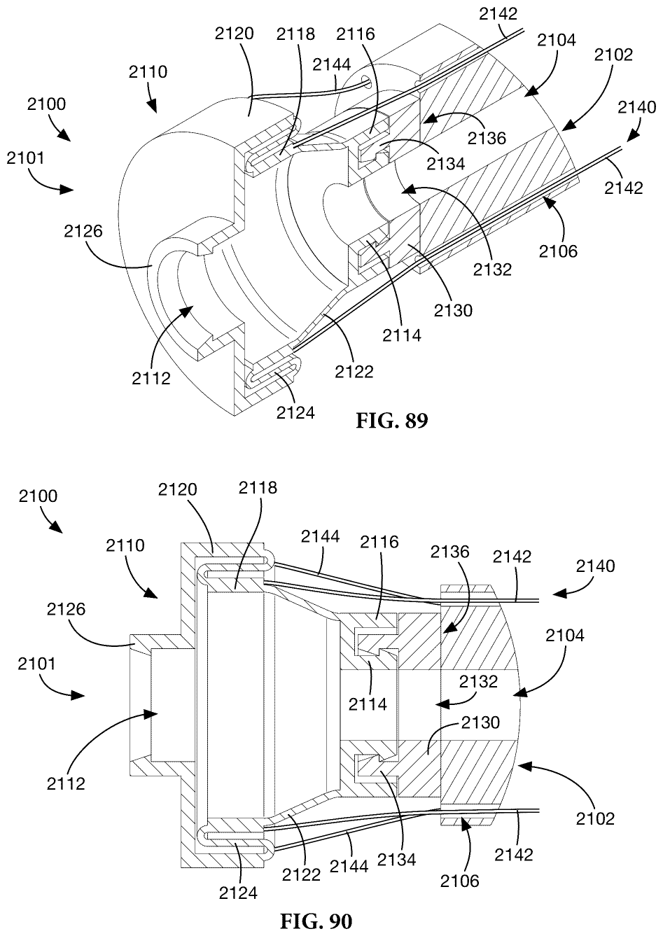

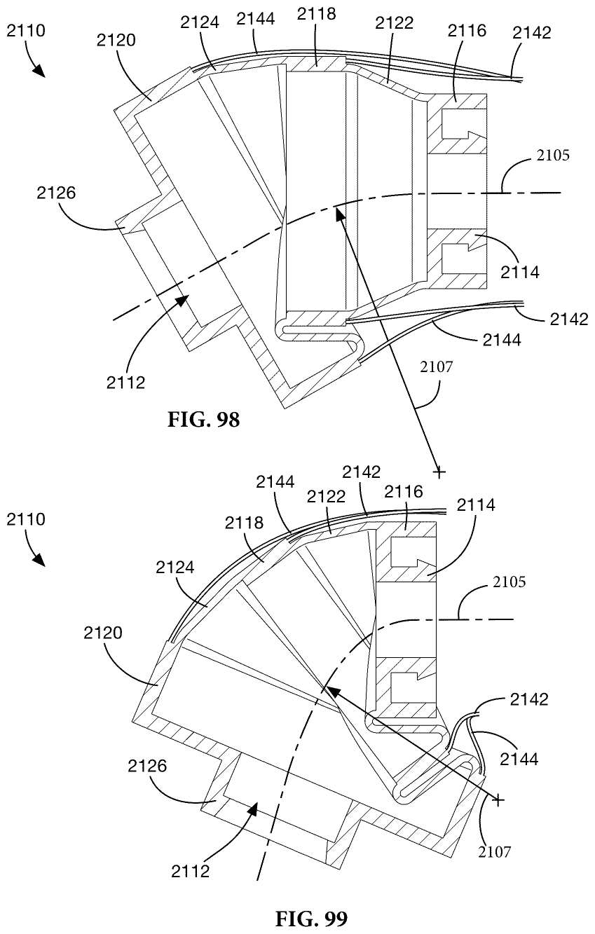

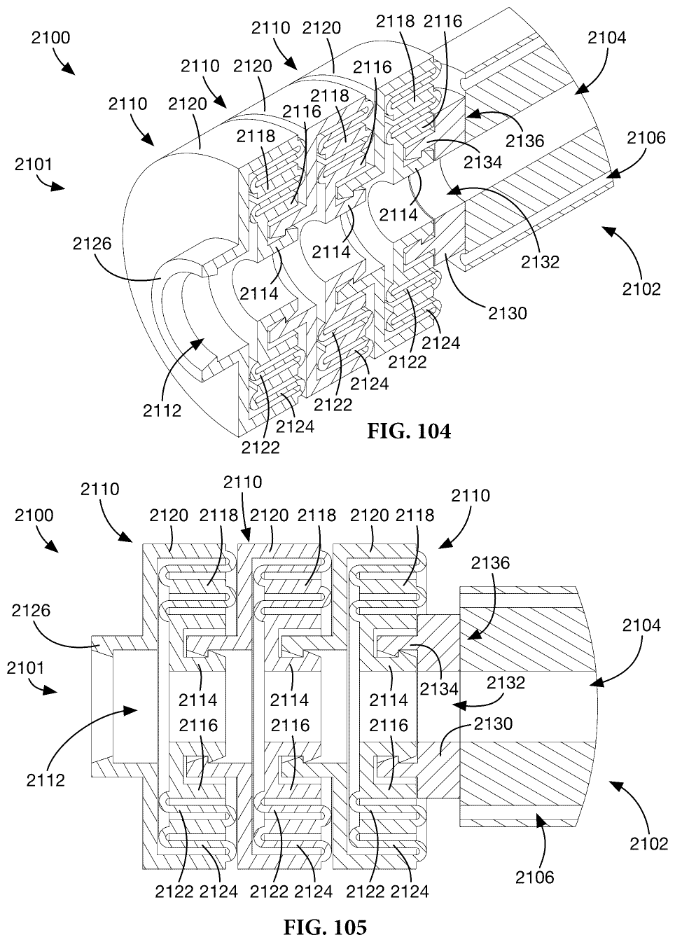

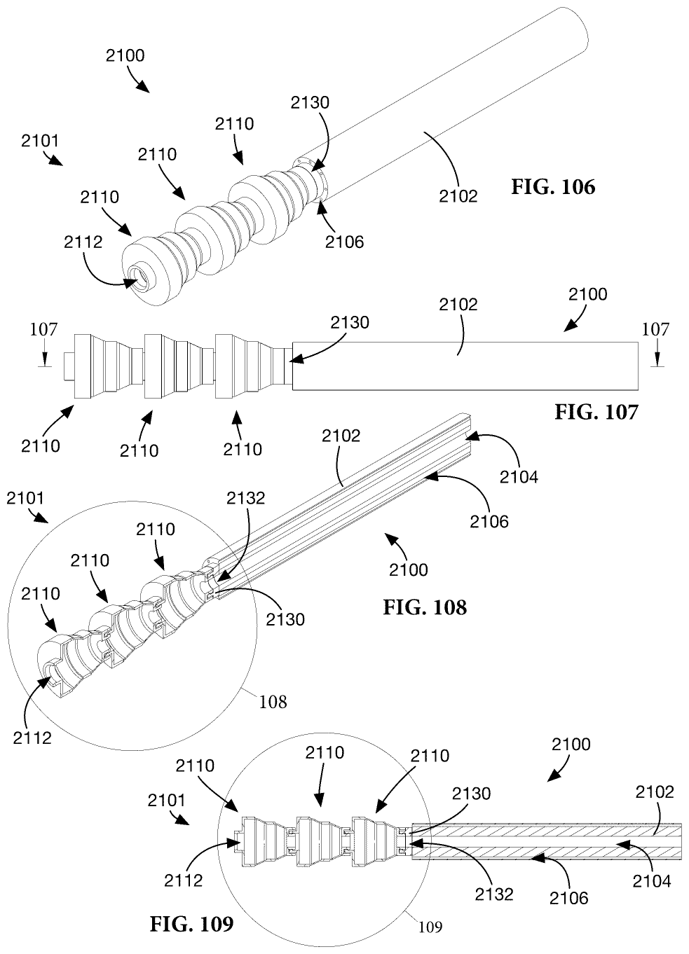

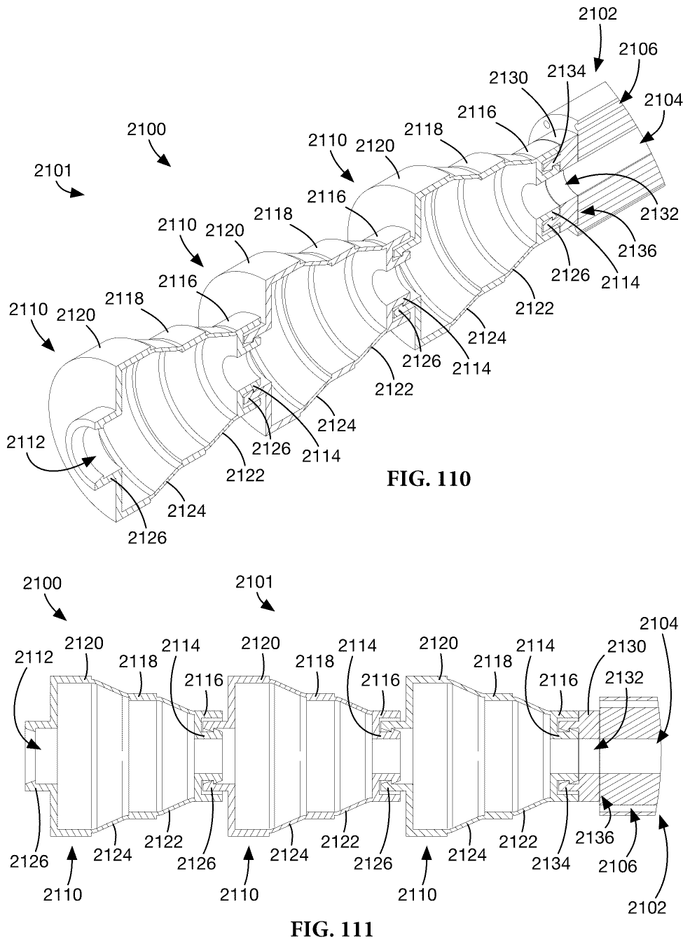

Referring now to FIGS. 74-115, an example steerable catheter 2100 is shown that includes a telescopic articulation device 2110 attached to or incorporated with a distal end 2101 of a delivery sheath 2102. The telescopic articulation device 2110 is attached to the distal end 2101 of the delivery sheath 2102 by way of a fitting 2130. However, the telescopic articulation device 2110 can be attached to the distal end 2101 of the delivery sheath 2102 in any manner or can be integrally formed with the delivery sheath 2102.

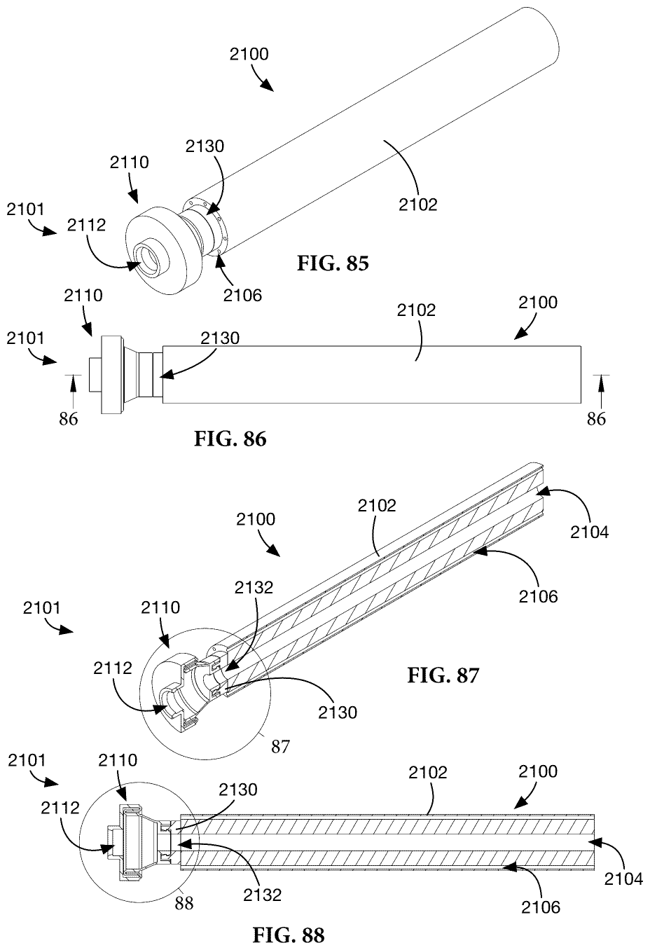

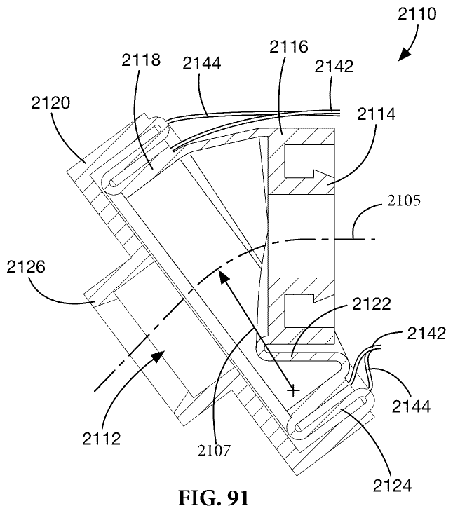

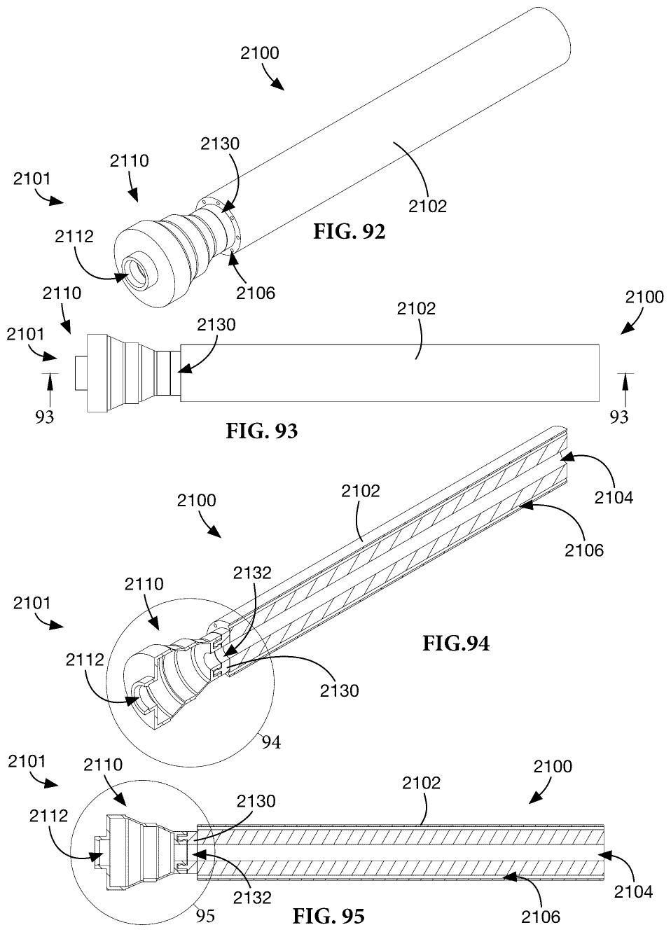

In some implementations, the telescopic articulation device 2110 can be manipulated from a collapsed condition (FIGS. 74-80), to a partially extended condition (FIGS. 85-91), and to a fully extended condition (FIGS. 92-99). In the partially and fully extended conditions the telescopic articulation device 2110 can be bent (FIGS. 91, 98, and 99) to facilitate implantation of a device or implant (e.g., an implantable prosthetic device, a prosthetic spacer device, a valve repair device, a valve replacement device, etc.). In the bent condition, a central path 2105 of the device 2110 has a bend radius 2107 that varies based on the extent to which the telescopic articulation device 2110 is extended and collapsed.

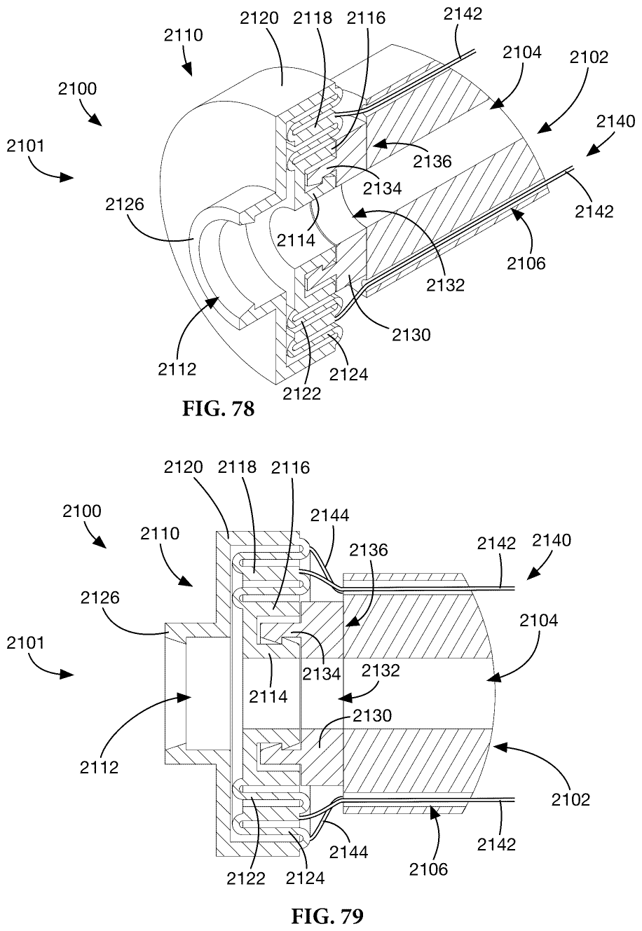

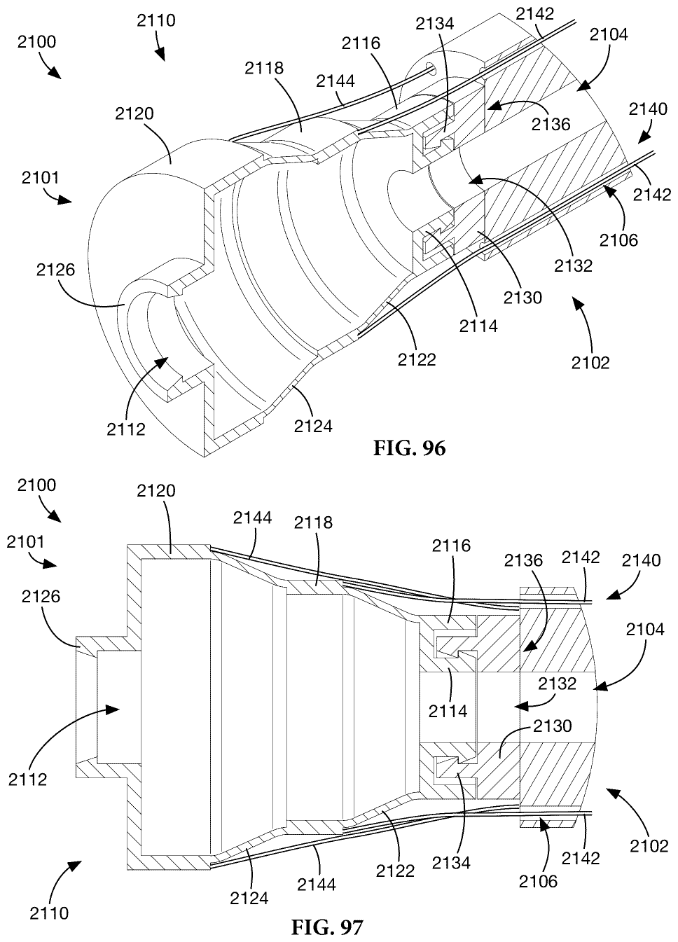

In some implementations, multiple telescopic articulation devices 2110 can be attached in series—i.e., end to end—as shown in FIGS. 96-111 to provide the distal end 2101 of the delivery sheath 2102 with a greater extension and bending range than that of a single telescopic articulation device 2110. In the extended condition the telescopic articulation device 2110 can be caused to bend or flex via the actuation of a plurality of actuation elements 2140 ((e.g., control wires, pull wires, lines, sutures, wires, rods, etc., see, e.g., FIGS. 78-79) arranged around the circumference of the catheter of the delivery system 200.

Referring now to FIGS. 74-80, an example telescopic articulation device 2110 is shown in a collapsed condition and attached to the distal end 2101 of the delivery sheath 2102 of the steerable catheter 2100. In some implementations, the delivery sheath 2102 includes a central lumen 2104 extending from a proximal end (not shown) to the distal end 2101 to enable delivery of a device or implant (e.g., an implantable prosthetic device, a prosthetic spacer device, a valve repair device, a valve replacement device, etc.) for implantation. In some implementations, a plurality of control lumens 2106 for steering or actuation elements 2140 (see, e.g., FIGS. 78-79) are arranged around the central lumen 2104.

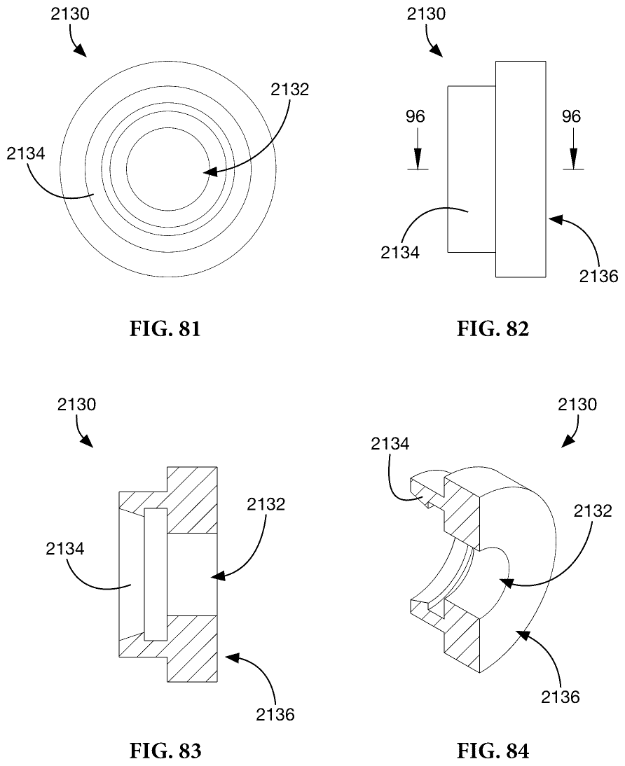

In some implementations, the telescopic articulation device 2110 is attached to the distal end 2101 of the delivery sheath 2102 via a fitting 2130, shown separated from the delivery sheath 2102 and telescopic articulation device 2110 in FIGS. 81-84. The fitting 2130 has a central lumen 2132 that is coaxially aligned with the central lumen 2104 of the delivery sheath 2102. In some implementations, the fitting 2130 connects to the telescopic articulation device 2110 at a distal connection end 2134 and to the delivery sheath at a proximal attachment portion 2136. In some implementations, the connection end 2134 is a female connector that is formed from an annular protrusion having a sloped inner diameter leading to an annular recess having a greater diameter than the sloped portion. In some implementations, the connection end 2134 is sufficiently flexible to allow a male connector (e.g., a proximal connector 2114 of the telescopic articulation device 2110) to be inserted into the connection end 2134 until the annular recess is engaged.

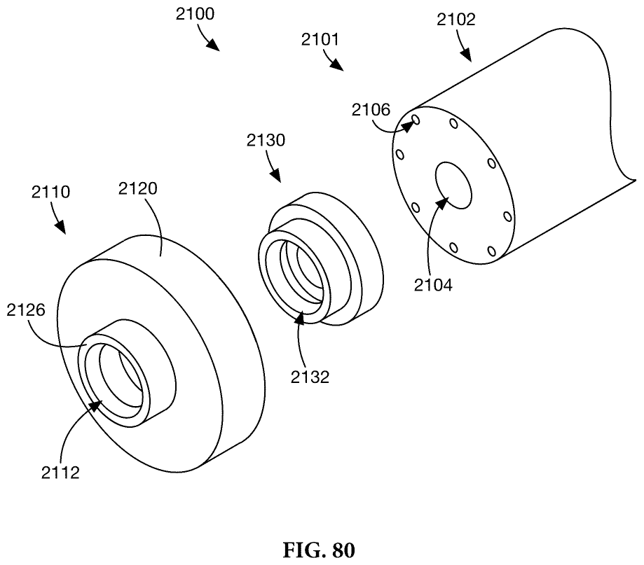

The fitting 2130 can be a separate component from the delivery sheath 2102—as is illustrated in FIG. 80 that shows an exploded view of the steerable catheter 2100—or can be integrally formed with the delivery sheath 2102. In particular, the distal end 2101 of the delivery sheath 2102 can be provided with a connection end 2134 for attaching to the telescopic articulation device 2110. The proximal attachment portion 2136 of the fitting 2130 can be any suitable connection for attaching the fitting 2130 to the delivery sheath 2102, such as, for example, a welded connection, a threaded connection, a swaged and/or other mechanical connection, an adhesive connection, or the like.

The telescopic articulation device 2110 also includes a central lumen 2112 that is coaxially aligned with the central lumen 2104 of the delivery sheath 2102 and the central lumen 2132 of the fitting 2130. In some implementations, the telescopic articulation device 2110 extends from the male proximal connector 2114 to a female distal connector 2126. In some implementations, the proximal connector 2114 is a male connector that corresponds to the female connector of the connection end 2134 of the fitting 2130—i.e., the proximal connector 2114 has an outwardly sloping end leading to an annular recess that has a smaller diameter than the sloping end. In some implementations, the male and female shapes of the proximal connector 2114 and the connection end 2134 can be swapped; that is, the proximal connector 2114 can be formed as a female connector and the connection end 2134 can be formed as a male connector.

In some implementations, the proximal connector 2114 can be pushed inside the connection end 2134 to snap the two components together. In some implementations, one or more relief cuts can be made in one or both of the proximal connector 2114 and the connection end 2134 to facilitate the engagement of the male and female connectors. In some implementations, the annular recesses of the proximal connector 2114 and the connection end 2134 enable the telescopic articulation device 2110 to rotate relative to the fitting 2130. In some implementations, the connection between the proximal connector 2114 and the connection end 2134 can be a fixed connection or can include features that prohibit relative rotation of the proximal connector 2114 and the connection end 2134.

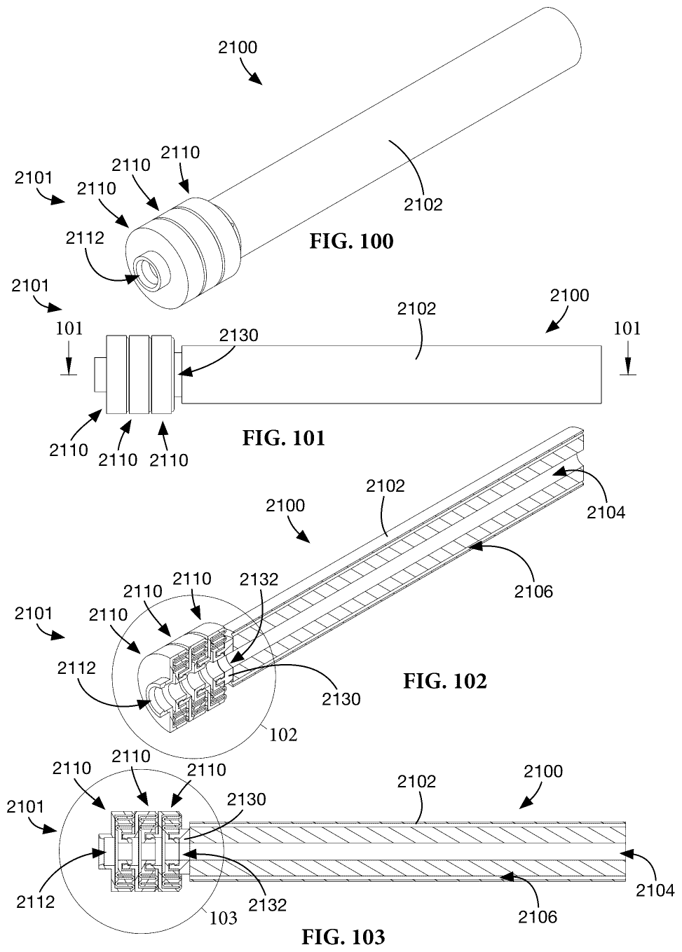

In some implementations, the distal connector 2126 is a female connector similar to the connection end 2134 of the fitting 2130 and is shaped to receive the male proximal connector 2114 of another telescopic articulation device 2110 (see, e.g., FIGS. 100-111). Consequently, the connection between the proximal connector 2114 and distal connector 2126 of two telescopic articulation devices 2110 shares the same characteristics as the connection between the proximal connector 2114 of the telescopic articulation device 2110 and the connection end 2134 of the fitting 2130.

In some implementations, a series of rigid and flexible portions connect the proximal connector 2114 to the distal connector 2126. In particular, the telescopic articulation device 2110 can include a first rigid ring 2116, a second rigid ring 2118, and a third rigid ring 2120. A first flexible hinge portion 2122 connects the first rigid ring 2116 to the second rigid ring 2118 and a second flexible hinge portion 2124 connects the second rigid ring 2118 to the third rigid ring 2120. The first rigid ring 2116 is rigidly connected to and surrounds the proximal connector 2114 and the third rigid ring 2120 is rigidly connected to the distal connector 2126. In some implementations, flexible hinge portions may not be included between the rings allowing the rings to be open therebetween (or have open sections/portions therein) and move more freely in various directions.

In some implementations, when the telescopic articulation device 2110 is in the extended condition the second rigid ring 2118 and the third rigid ring 2120 divide the telescopic articulation device 2110 longitudinally into sections that can be independently actuated by the actuation elements 2140. In some implementations, the bending characteristics from one section of the telescopic articulation device 2110 to another can be different. While three rigid rings 2116, 2118, 2120 are shown, the telescopic articulation device 2110 can include any suitable number of rigid rings, such as, for example, 2, 3, 4, 5, or more rigid rings that can correspond to a similar number of articulable sections of the telescopic articulation device 2110.

In some implementations, the second rigid ring 2118 has a smaller diameter than the third rigid ring 2120 so that the second rigid ring 2118 can nest inside of the third rigid ring 2120 when the telescopic articulation device 2110 is in the collapsed condition. The first rigid ring 2116 has a smaller diameter than the second rigid ring 2118 so that the first rigid ring 2116 can nest inside of the second rigid ring 2120 when the telescopic articulation device 2110 is in the collapsed condition. Thus, the first rigid ring 2116 and the second rigid ring 2118 both nest inside of the third rigid ring 2120 when the telescopic articulation device 2110 is in the collapsed condition. Alternatively, the second rigid ring 2118 and the third rigid ring 2120 can be smaller than the first rigid ring 2116 so that the second rigid ring 2118 and the third rigid ring 2120 nest inside of the first rigid ring 2116.

In some implementations, the first flexible hinge portion 2122 extends from a distal end of the first rigid ring 2116 to a proximal end of the second rigid ring 2118. In some implementations, the second flexible hinge portion 2124 extends from a distal end of the second rigid ring 2118 to a proximal end of the third rigid ring 2120. In some implementations, the first flexible hinge portion 2122 and the second flexible hinge portion 2124 are formed from a flexible and elastic material that allows portions of first flexible hinge portion 2122 and the second flexible hinge portion 2124 to stretch and expand so that the second rigid ring 2118 and the third rigid ring 2120 can move from the collapsed condition (FIGS. 74-80) to the fully extended condition (FIGS. 92-97). In some implementations, the second and third rigid rings 2118, 2120 can be moved to the extended condition simultaneously or can be moved one at a time so that the telescopic articulation device 2110 moves into a partially extended condition (FIGS. 85-90) before being further extended to the fully extended condition. When multiple telescopic articulation devices 2110 are connected in series, as is shown in FIGS. 100-111, any number of the sections of the connected telescopic articulation devices 2110 can be extended so that the length of the connected telescopic articulation devices 2110 can vary greatly from the collapsed to the fully extended condition.

The minimum length of the telescopic articulation device 2110 in the collapsed condition is determined by adding together the length of the distal connector 2126, the first rigid ring 2116, the second rigid ring 2118, and the third rigid ring 2120 and then subtracting the length of the first flexible hinge portion 2122 and the second flexible hinge portion 2124. In some implementations, the proximal connector 2114 is nested within the first rigid ring 2116 and does not impact the overall length of the telescopic articulation device 2110. The maximum length of the telescopic articulation device 2110 in the extended condition is determined by adding together the length of the first rigid ring 2116, the second rigid ring 2118, the third rigid ring 2120, the first flexible hinge portion 2122, and the second flexible hinge portion 2124.

In some implementations, the minimum outer diameter of the telescopic articulation device 2110 is determined by adding together the diameter of the central lumen 2112 and the thickness of the first rigid ring 2116, the second rigid ring 2118, the third rigid ring 2120, the first flexible hinge portion 2122, and the second flexible hinge portion 2124. The third rigid ring 2120 is arranged at the outer diameter of the telescopic articulation device 2110 so that the outer diameter of the third rigid ring 2120 is the outer diameter of the telescopic articulation device 2110. Alternatively, the first rigid ring 2116 can be arranged at the outer diameter of the telescopic articulation device 2110.

In some implementations, the telescopic articulation device 2110 can be formed by molding the rigid rings and flexible hinge portions in a single molding operation via injection molding or other suitable molding techniques, with the relative rigidity or flexibility of the rigid rings and flexible hinge portions being determined by the thickness and other geometric properties of the rigid rings and flexible hinge portions. In some implementations, the rigid rings can also be formed from a more rigid material and placed into a mold for forming the telescopic articulation device 2110 from a more flexible material so that the flexible hinge portions are more flexible than the rigid rings. In some implementations, the telescopic articulation device 2110 can also be formed from a tube of material that is formed into a roughly conical shape or from a sheet of material that is rolled and welded or otherwise joined along a seam. In some implementations, the rigid rings can be attached to the tube or sheet of material by any suitable attachment means, such as, for example, welding, an adhesive, mechanical fastening, or the like.

In some implementations, the example telescopic articulation device 2110 described herein can transition between the collapsed condition and the expanded condition in a wide variety of ways. In some implementations, the first flexible hinge portion 2122 and the second flexible hinge portion 2124 can include biasing members (e.g. springs, tensioning members, tension lines, shape memory material, elastic bands, stiffening members, wires, coils, curved components, etc.) or be formed from a shape-memory material such that the telescopic articulation device 2110 is biased in the expansion direction and the application of a tension force via the actuation element(s) 2140 is required to retain the telescopic articulation device 2110 in the collapsed condition.

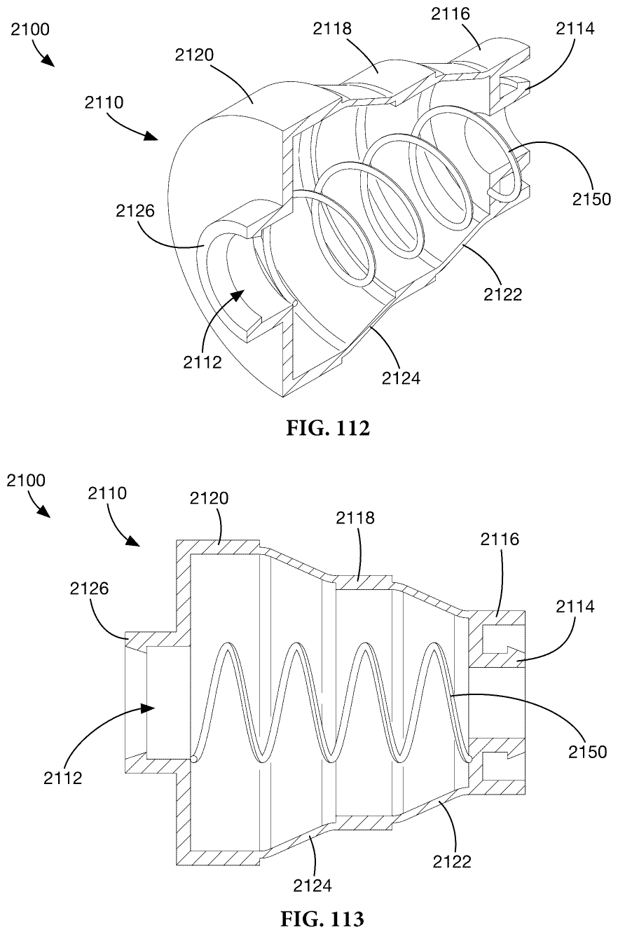

Referring now to FIGS. 112-113, a biasing member 2150 can be arranged between the proximal connector 2114 and the distal connector 2126 so that the telescopic articulation device 2110 is biased in the expansion direction. The biasing member 2150 can take on a wide variety of forms, such as a spring (as shown), a plurality of springs, an elastic member, tensioning member, tension line, compression member, shape memory material, elastic band, stiffening member, wire, coil, curved component, and/or the like. When a spring is used as the biasing member 2150 the spring can be a conical spring that collapses to a substantially flat profile in the collapsed or compressed condition to reduce the longitudinal length of the telescopic articulation device 2110 in the collapsed condition.

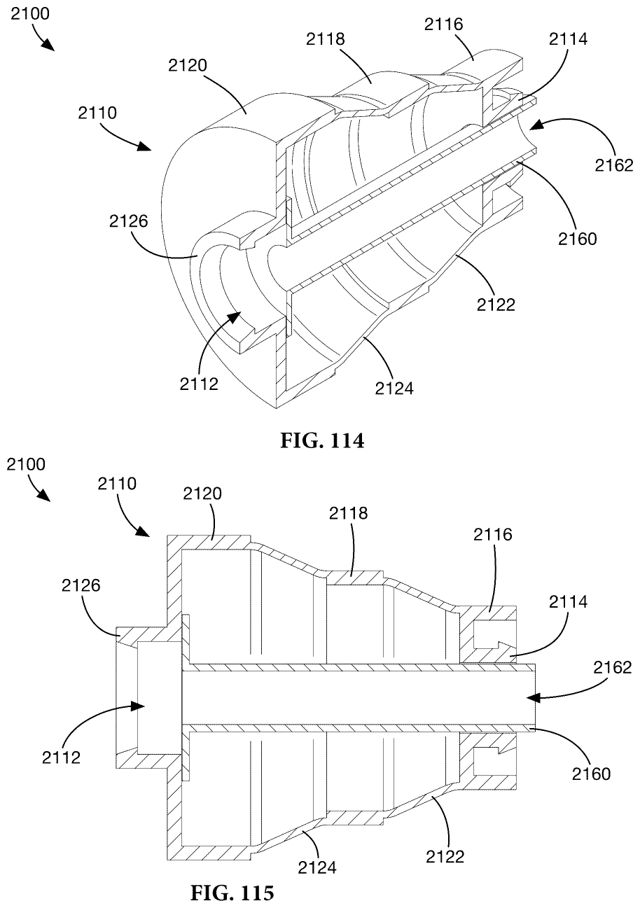

Referring now to FIGS. 114-115, an optional pushing member 2160 (e.g., rod, shaft, etc.) extends through the central lumen 2112 of the telescopic articulation device 2110 from the proximal connector 2114 to the distal connector 2126. The pushing member 2160 can be actuated to push the distal connector 2126 away from the proximal connector 2114 to extend the telescopic articulation device 2110 from the collapsed condition to the extended condition. The pushing member 2160 can extend proximally to the handle or other actuation mechanism and includes a central lumen 2162 to allow the delivery of an implantable device through the pushing member 2160. The pushing member 2160 can also be flexible to accommodate the steering of the telescopic articulation device 2110 while the pushing member 2160 is in the extended condition.

In some implementations, when the telescopic articulation device 2110 is in the partially or fully extended condition tension can be applied to the actuation elements 2140 to steer the telescopic articulation device 2110 or to retract the telescopic articulation device 2110 into the collapsed condition. In some implementations, the actuation elements 2140 extend through the control lumens 2106 from proximal ends that are attached to a handle or one or more other steering systems or mechanisms (not shown) to distal ends that exit the control lumens 2106 of the delivery sheath 2102 to engage the telescopic articulation device 2110. In some implementations, the control lumens 2106 can extend through the fitting 2130 or the fitting 2130 can be small enough in diameter to avoid obstructing the control lumens 2106.

In some implementations, the actuation elements 2140 are arranged into two groups: a first group 2142 for articulating the second rigid ring 2118 and a second group 2144 for articulating the third rigid ring 2120. In some implementations, the actuation elements 2140 of the first group 2142 extend from the control lumens 2106 to attach to the second rigid ring 2118. In some implementations, the actuation elements 2140 of the second group 2144 extend from the control lumens 2106 to attach to the third rigid ring 2120. In some implementations, the actuation elements 2140 of the second group 2144 can also extend through openings (not shown) in the second rigid ring 2118.

In some implementations, the actuation elements 2140 of the first group 2142 and of the second group 2144 extend from the eight control lumens 2106 (seen clearly in FIG. 80) in an alternating fashion so that the first group 2142 and the second group 2144 each include four actuation elements 2140. In some implementations, the actuation elements 2140 in each of the first group 2142 and the second group 2144 are radially spaced apart from each other by about 90 degrees so that the four actuation elements 2140 in each group are evenly spaced around the circumference of the delivery sheath 2102. In some implementations, the first group 2142 and the second group 2144 are also offset from each other by about 45 degrees so that the control lumen 2106 for the first group 2142 and the second group 2144 can also be evenly spaced around the circumference of the delivery sheath 2102.

In some implementations, the first group 2142 and the second group 2144 can include two or three actuation elements 2140 that are radially spaced apart from each other by about 180 degrees or by about 120 degrees, respectively.

In some implementations, applying tension to the actuation elements 2140 causes the attached second rigid ring 2118 or third rigid ring 2120 to move or tilt in the direction of the net tension force applied to the rigid ring 2118, 2120. Consequently, the telescopic articulation device 2110 is caused to flex or bend toward the applied force. Bending forces applied to one side of the telescopic articulation device 2110 via one of the four actuation elements 2140 in one of the first group 2142 and the second group 2144 can be counteracted by forces applied to one or more opposing actuation elements 2140 in the first group 2142 and the second group 2144.

In some implementations, providing four actuation elements 2140 in each of the first group 2142 and the second group 2144 enables each of the second rigid ring 2118 and the third rigid ring 2120 to be articulated in any direction around the delivery sheath 2102 by varying the amount of tension applied to each of the actuation elements 2140. In particular, the direction of the bend in the telescopic articulation device 2110 depends on the proportional distribution of tension forces in each of the actuation elements 2140 of the first group 2142 or the second group 2144. That is, the relative proportion of tension applied to each of the actuation elements 2140—independent of the amount of tension applied—determines bend direction. The amount of tension applied to the actuation elements 2140, however, is directly related to the magnitude of the bend in the telescopic articulation device 2110; the greater the tension imbalance the greater the bend magnitude (i.e., the tighter or smaller the bend radius). Consequently, the steerable catheter 2100 can be articulated into a wide variety of positions.

In some implementations, applying tension to two opposing actuation elements 2140 or to all of the actuation elements 2140 in the first group 2142 retracts the second rigid ring 2118 in the proximal direction and causes the first flexible hinge portion 2122 to collapse. In some implementations, applying tension to two opposing actuation elements 2140 or to all of the actuation elements 2140 in the second group 2144 retracts the third rigid ring 2120 in the proximal direction and causes the second flexible hinge portion 2124 to collapse. Tension can be applied in this way to retract the telescopic articulation device 2110 from the extended position back to the collapsed condition.

In some implementations, the actuation elements 2140 can be connected to one or more steering elements of a steering system or mechanism (e.g., an assembly of components that work together to steer; steering elements and actuation elements; a knob and one or more pull wires; etc.), such as any of the example steering systems or mechanisms disclosed herein, that is arranged in a handle (not shown) at a proximal end of the catheter. For example, a first steering system or mechanism can be used to control the bending of the second rigid ring 2118 and a second steering system or mechanism can be used to control the bending of the third rigid ring 2120. Optionally, a single steering system or mechanism can control both the second rigid ring 2118 and the third rigid ring 2120. The steering systems or mechanism(s) can be arranged in a single handle or in multiple handles such that each handle contains a single steering system or mechanism.

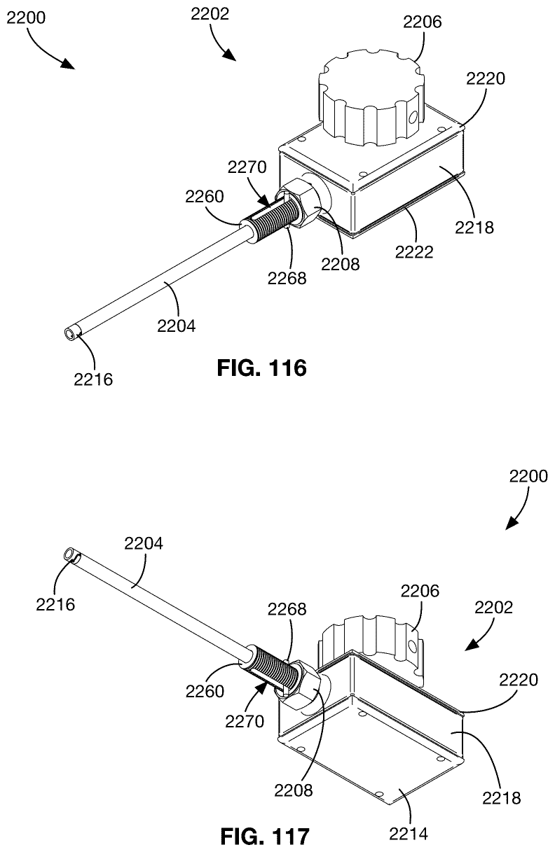

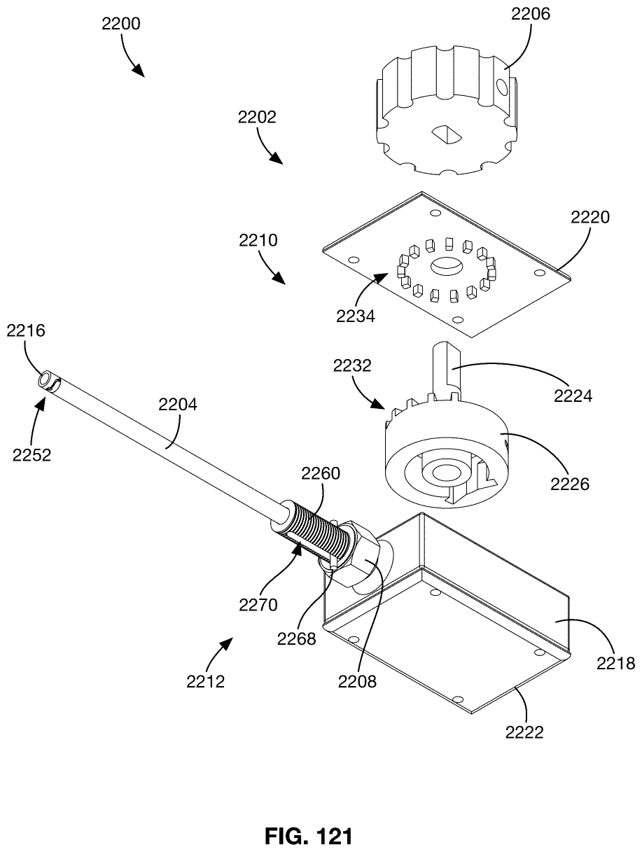

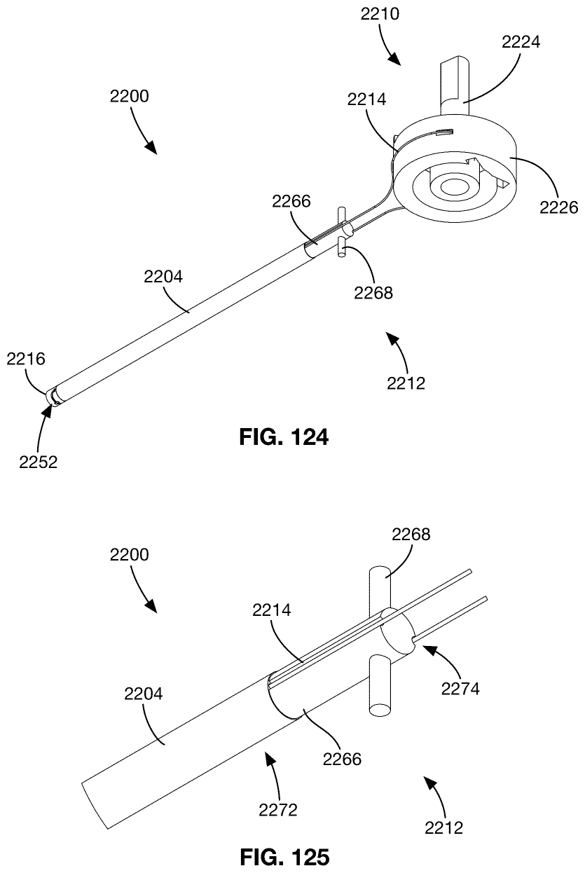

Referring now to FIGS. 116-125, an example control handle 2202 for a delivery system 2200 is shown. In some implementations, the control handle 2202 is attached to a proximal end of a catheter shaft 2204 and includes at least one control member 2206, 2208 (e.g., knob, handle, buttons, switches, etc.) for actuating at least one steering system 2210, 2212 incorporated into the control handle 2202. In some implementations, the control handle 2202 is attached to a proximal end of a catheter shaft 2204 and includes first and second control members 2206, 2208 (e.g., knob, handle, buttons, switches, etc.) for actuating first and second steering systems 2210, 2212 incorporated into the control handle 2202. Additional control members and steering systems are also possible, e.g., 3, 4, 5, 6, etc.

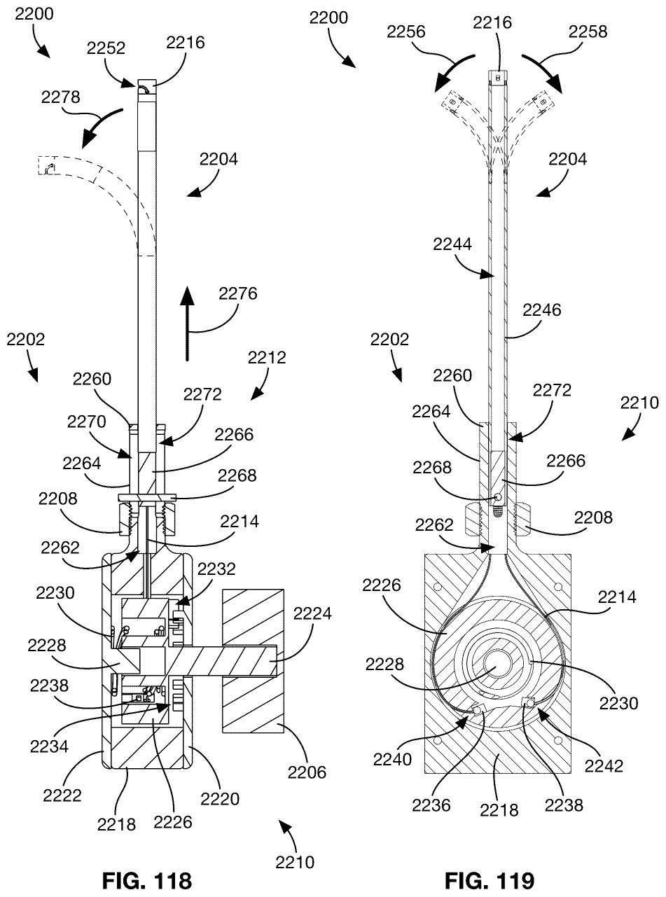

In some implementations, at least one control member 2206, 2208 actuates at least one steering system 2210, 2212 to enable the control of at least one actuation element 2214 (e.g., control wires, pull wires, lines, sutures, wires, rods, etc., and can be the same as or similar to those of FIGS. 118 and 122-125) that extends down the catheter shaft 2204 to manipulate a distal fitting 2216 (e.g., pull ring, connection, strip, groove, etc.) and/or distal end portion of the delivery system 2200.

In some implementations, first and second control members 2206, 2208 actuate first and second steering systems 2210, 2212 that enable the control of an actuation element 2214 (e.g., control wires, pull wires, lines, sutures, wires, rods, etc., and can be the same as or similar to those of FIGS. 118 and 122-125) that extends down the catheter shaft 2204 to manipulate a distal fitting 2216 (e.g., pull ring, connection, strip, groove, etc.) and/or or distal end portion of the delivery system 2200, respectively.

In some implementations, there is a single actuation element. In some implementations, there is a single actuation element that forms an open loop. In some implementations, there is a single actuation element that forms a closed loop. In some implementations, there are multiple actuation elements. A variety of configurations of one or more actuation elements can be arranged to accomplish the necessary movements and controls herein.

In some implementations, the control handle 2202 has a somewhat rectangular box shape but can take on a wide variety of shapes (e.g., circular, oval, columnar, ovoid, symmetrical, asymmetrical, etc.). Varying arrangements of the control member or control members (e.g., the first and second control members 2206, 2208) are also possible.

Control handles disclosed herein, such as the control handle 2202 of the delivery system 2200 can have a variety of configurations, shapes, and sizes for various types of steering systems and steering elements located inside the handle and depending on the use of the catheter.

In some implementations, the control handle 2202 includes a housing 2218 having a top cover 2220 and a bottom cover 2222. In some implementations, the first control member 2206 is a rotatable knob that engages the first steering system 2210. Referring to FIGS. 118 and 119, the first steering system 2210 can include a shaft 2224 of a hub 2226 that is arranged inside the housing 2218 between the top cover 2220 and the bottom cover 2222.

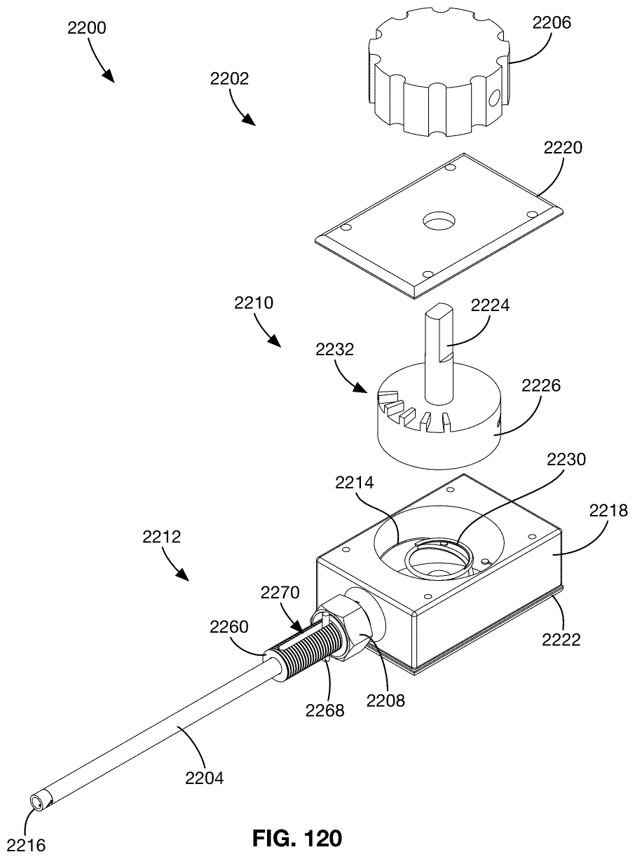

In some implementations, the hub 2226 engages and rotates around a boss 2228 extending from the bottom cover 2222. An optional biasing member 2230 can be arranged between the hub 2226 and the bottom cover 2222 to bias the hub 2226 towards the top cover 2220. The biasing member 2230 (e.g., spring, elastic member, coil, tension line, etc.) can take on a wide variety of forms, such as, for example, a spring like the coil spring shown in FIG. 120, a tension member connecting to the top cover 2220, a coil spring between the top cover 2220 and the first control member 2206, or the like.

In some implementations, the side of the hub 2226 facing the top cover 2220 includes an optional moveable locking portion 2232 that engages an optional fixed locking portion 2234 of the top cover 2220 to fix the rotational position of the hub 2226 relative to the housing 2218. In the illustrated example, the moveable locking portion 2232 includes a plurality of teeth extending towards the top cover 2220 to engage a corresponding plurality of teeth extending from the top cover 2220 towards the hub 2226. The biasing member 2230 pushes the hub 2226 toward the top cover 2220 to cause the moveable locking portion 2232 to engage the fixed locking portion 2234 and lock the rotational position of the hub 2226.

In some implementations, applying pressure to the first control member 2206 towards the housing 2218 compresses the biasing member 2230 so that the moveable locking portion 2232 disengages from the fixed locking portion 2234 to allow the hub 2226 to rotate as the first control member 2206 is rotated. When the pressure applied to the first control member 2206 is released, the biasing member 2230 moves the moveable locking portion 2232 into engagement with the fixed locking portion 2234 of the top cover 2220 to lock the position of the hub 2226 relative to the housing 2218.

In some implementations, the hub 2226 includes a first grasping element 2236 (e.g. clamp, fastener, such as a set screw, crimp, connector, etc.) and a second grasping element 2238 (e.g. clamp, fastener, such as a set screw, crimp, connector, etc.) for securing the actuation element(s) 2214 (e.g., control wires, pull wires, lines, sutures, wires, rods, etc.) to the hub 2226. The first and second grasping elements 2236, 2238 can take a wide variety of forms, such as, for example, a fastener, such as a set screw, a clamp, a clip, a snap-fit, a recess, any grasping element disclosed herein, etc. In some implementations, the first and second grasping elements 2236, 2238 can be formed integrally with the hub 2226 or can be attached to the hub 2226 via adhesives, fasteners, or any other securing means. In some implementations, the first grasping element 2236 secures a first end 2240 of the actuation element 2214 to the hub 2226 and the second grasping element 2238 secures the second end 2242 of the actuation element 2214 to the hub 2226. In other implementations, the two sides of the actuation elements are two separate pieces.

In some implementations, the catheter shaft 2204 can include an optional central lumen 2244. Implantable devices and other devices can be delivered through the lumen, shafts or catheters used to deliver implantable devices can extend through the lumen and/or control elements (e.g., control wires, pull wires, lines, sutures, wires, rods, etc.) for controlling implantable devices can extend through the lumen.

In some implementations, a wall 2246 of the catheter shaft 2204 includes a first lumen 2248 and a second lumen 2250 through which the actuation element 2214 passes. In some implementations, the actuation element 2214 extends from the first end 2240 that is secured by the first grasping element 2236, out of the housing 2218, through the first lumen 2248 of the catheter shaft 2204 to the distal fitting 2216, through the second lumen 2250 of the catheter shaft 2204, and to the second end 2242 that is secured by the second grasping element 2238. In some implementations fewer grasping elements (e.g., one to grasp multiple portions of an actuation element) or more grasping elements can be used.

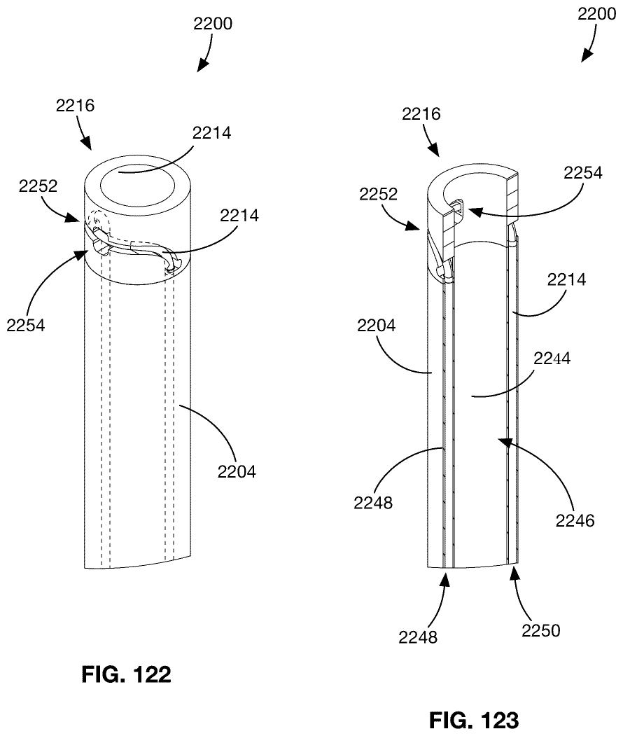

Referring to FIGS. 122 and 123, in some implementations, in the distal fitting 2216, the actuation element 2214 is routed through a connecting groove 2252 extending between the first lumen 2248 and the second lumen 2250 and along the outer diameter of the distal fitting 2216. Consequently, the connecting groove 2252 directs the actuation element 2214 around the central lumen 2244 to one side of the catheter shaft 2204, such as, for example, a side opposite the first control member 2206 or on the same side as the first control member 2206. In some implementations, the connecting groove 2252 can optionally include a securing portion 2254 for receiving a ferrule (not shown) that is fixedly secured to the actuation element 2214 in the location of the securing portion 2254 when the catheter shaft 2204 is in a straight configuration. Other arrangements are possible, e.g., a lumen or other pathway instead of a groove, in a variety of types of fittings, including a fitting integrally formed as part of the catheter shaft, etc.

In some implementations, the tight radius of the connecting groove 2252 and the resulting friction between the actuation element 2214 and the connecting groove 2252 of the distal fitting 2216 prohibit or inhibit sliding of the actuation element 2214 through the connecting groove 2252. In some implementations, pulling on either of the first end 2240 and the second end 2242 of the actuation element 2214 (e.g., by rotating the hub 2226) causes the catheter shaft 2204 to bend in the direction of the first or second end 2240, 2242 of the actuation element 2214 that is pulled. In some implementations, bending of the catheter shaft 2204 can also be carried out via the attachment of a ferrule to the actuation element 2214 in the location of an optional securing portion 2254 of the distal fitting 2216 so that tension applied to one of the first and second ends 2240, 2242 of the actuation element is transmitted to the distal fitting 2216 as a bending force.

In some implementations, during operation of the control handle 2202, rotating the first control member 2206 engages the first steering system 2210 to rotate the hub 2226 in a counterclockwise direction pulls on the first end 2240 of the actuation element 2214 in a proximal direction while simultaneously letting the second end 2242 of the actuation element 2214 move in the distal direction. As a result, the catheter shaft 2204 is caused to bend in a first or leftward direction 2256, as is shown in broken lines in FIG. 119. On the contrary, rotating the first control member 2206 and the hub 2226 in a clockwise direction pulls on the second end 2242 of the actuation element 2214 in a proximal direction while simultaneously letting the first end 2240 of the actuation element 2214 move in the distal direction. In some implementations, the catheter shaft 2204 is thereby caused to bend in a second or rightward direction 2258, as is shown in broken lines in FIG. 119. Thus, the first control member 2206 of the control handle 2202 described herein can be rotated to cause bending of the catheter shaft 2204 in two different directions. In one implementation, the location and degree of the bending of the catheter shaft 2204 is predetermined via the construction of the catheter shaft 2204, such as the catheter shafts disclosed herein.

In some implementations, to facilitate rotation of the first control member 2206, the first control member 2206 is pressed downward towards the housing 2218 to disengage the moveable locking portion 2232 of the hub 2226 from the fixed locking portion 2234 of the top plate or cover 2220. In some implementations, downward pressure is applied to the first control member 2206 during rotation until the catheter shaft 2204 is bent to a desired degree at which time the pressure is released to allow the moveable locking portion 2232 to engage the fixed locking portion 2234. In some implementations, while the fixed locking portion 2234 engages the moveable locking portion 2232, the hub 2226 is prohibited from rotating and the bent condition of the catheter shaft 2204 is maintained without further input from the operator of the control handle 2202. In some implementations, the catheter shaft 2204 can be returned to a straight condition by again pressing on the first actuation or control member 2206 to release the moveable locking portion 2232 from the fixed locking portion 2234 so that the hub 2226 can be rotated to straighten the catheter shaft 2204.

The second control member 2208 can take a wide variety of different forms. In some implementations, the second control member 2208 can be moved distally and proximally relative to the control handle 2202. For example, the second control member 2208 can comprise a threaded element, such as a threaded knob, the illustrated nut, etc. that can be moved distally and proximally along a neck 2260 of the control handle 2202 to engage the second steering system 2212. In some implementations, the second control member 2208 travels distally and proximally along a threaded outer diameter 2264 of the neck 2260 via rotation of the second control member 2208.

In some implementations, the neck 2260 has a central opening 2262 for carrying an actuation slider 2266 that is moved within the central opening 2262 by the engagement of a driving pin 2268 that extends through a slot 2270 in the neck 2260 to be engaged by the second control member 2208. In some implementations, the actuation slider 2266 engages and/or is connected to a proximal end 2272 of the catheter shaft 2204. Clearance channels 2274 (see FIG. 125) along the actuation slider 2266 provide a path for the actuation element 2214 to pass by the actuation slider 2266 and into the first lumen 2248 and the second lumen 2250 of the catheter shaft 2204.

In some implementations, during operation of the control handle 2202, the second control member 2208 can be rotated to translate the driving pin 2268 and the connected actuation slider 2266. When the actuation slider 2266 is in engagement with the proximal end 2272 of the catheter shaft 2204, further actuation of the second control member 2208 applies pressure to the catheter shaft 2204 in the distal direction 2276. As a result, the catheter shaft 2204 is compressed between the actuation slider 2266 and the distal fitting 2216 that is maintained at a fixed distance from the control handle 2202 by the actuation element 2214.

In some implementations, the routing of the actuation element 2214 through the connecting groove 2252 of the distal fitting 2216 offsets the compression force applied to the catheter shaft 2204 by the actuation slider 2266 to the side of the connecting groove 2252.

Referring to FIG. 118, in some implementations, the offset force causes the catheter shaft 2204 to bend in a third or downward 2278 direction that is different from the first direction 2256 and the second direction 2258. When pressure on the catheter shaft 2204 is released by moving the second control member 2208 in a proximal direction, the catheter shaft 2204 straightens and pushes the actuation slider 2266 in the proximal direction. In some implementations, the driving pin 2268 remains in contact with the second actuation or control member 2208.

The first and second control members 2206, 2208 can be actuated sequentially or simultaneously—that is, the catheter shaft 2204 can be bent to the left or right and then down, or down and then left or right, or simultaneously left and down or right and down. In some implementations, the moveable and fixed locking portions 2232, 2234 of the control handle 2202 enables the rotational position of the first control member 2206 and, consequently, the magnitude of the bending of the catheter shaft 2204 in the left direction 2256 or the right direction 2258 to be fixed without requiring the physician operating the control handle 2202 to continue to provide actuation force to the first control member 2206. Similarly, the position of the second control member 2208 along the neck 2260 can be maintained without input from the operator when the second control member 2208 is a nut or other internally threaded component and the neck 2260 includes a threaded outer diameter 2264. That is, the magnitude of bending of the catheter shaft 2204 in the third direction 2278 can be set at a desired bend angle prior to the actuation of the first control member 2206. Once the bend angle of the catheter shaft 2204 is set by actuation of the first and second control members 2206, 2208 no further input is required by the operator until the operator desires to straighten the catheter shaft 2204.

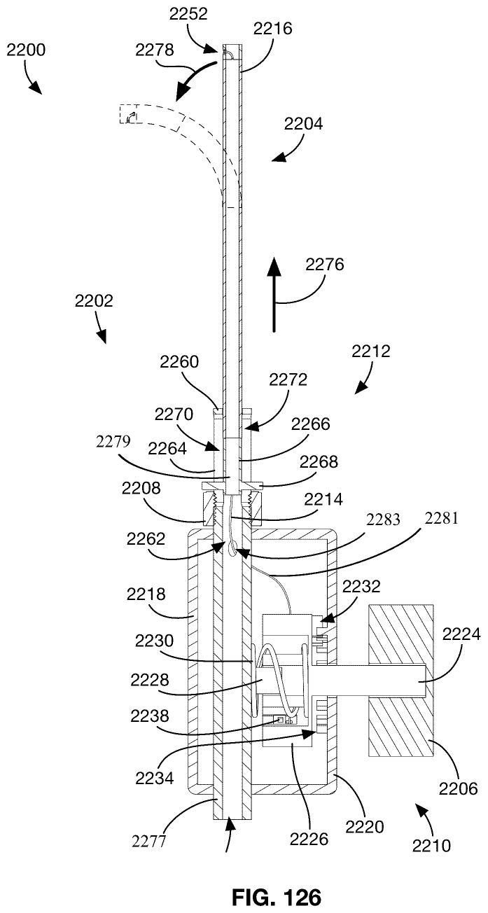

FIG. 126 shows an example of a control handle that is similar to the example of FIG. 116 that is configured to allow another catheter(s), guidewire, pusher, device, implant, etc. to pass through the control handle and attached catheter. In the FIG. 126 example, the housing 2218 is enlarged and the hub 2226 is shifted upward to make room for insertion of a catheter(s), guidewire, pusher, device, implant, etc.

In some implementations, an optional conduit 2277 extends through the housing to the neck 2260. In some implementations, the optional conduit 2277 can provide a sealed path to the slider 2266. In some implementations, the slider 2266 includes a bore 2279 that extends between the catheter shaft 2204 and the conduit 2277. The driving pin 2268 has two portions that extend from opposite sides of the slider 2266, instead of a single, solid pin that extends through the center of the slider. In some implementations, the driving pin 2268 can be integrally formed with the slider 2266 or can be separate pieces that are attached to the slider. In some implementations, the actuation element 2214 includes a curved portion 2281 that extends from the hub 2226 to the catheter shaft 2204. In some implementations, the curved portion 2281 compensates for the offset between the hub 2226 and the catheter shaft 2204.

In some implementations, the optional conduit 2277 can include an optional hole or passage 2283 to allow the actuation element 2214 to extend from the hub 2226 to the catheter shaft 2204. In the FIG. 126 example, another catheter(s), guidewire, pusher, device, implant, etc. can pass through the conduit 2277, the bore 2279 of the slider 2266, and through the catheter shaft 2204. The control handles 2202 of the FIG. 126 and FIG. 116 examples can otherwise operate in the same or substantially the same manner.

Any of the systems, devices, apparatuses, components, etc. herein can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

While various inventive aspects, concepts and features of the disclosures may be described and illustrated herein as embodied in combination in the examples, these various aspects, concepts, and features may be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the implementations in the specification.

What is claimed is:

1. A catheter assembly for delivering a medical device to a desired location, the catheter assembly comprising:
a catheter comprising a shaft extending from a proximal end to a distal end;
a telescopic articulation device disposed at the distal end of the shaft, wherein the telescoping articulation device is movable between a compressed configuration and an extended configuration;
at least one actuation element extending through the catheter, wherein the at least one actuation element has a distal end that is attached to the telescopic articulation device; and
wherein tension applied to the at least one actuation element steers the telescopic articulation device.

2. The catheter assembly of claim 1, wherein the telescopic articulation device comprises:
a first rigid ring and a second rigid ring, wherein the first rigid ring fits concentrically within the second rigid ring; and
a flexible hinge portion extending from a distal end of the first rigid ring to a proximal end of the second rigid ring.

3. The catheter assembly of claim 2, wherein the flexible hinge portion fits concentrically between the first rigid ring and the second rigid ring when the telescopic articulation device is in the compressed configuration.

4. The catheter assembly of claim 3, wherein the telescopic articulation device further comprises:
a third rigid ring, wherein the first rigid ring and the second rigid ring fit concentrically within the third rigid ring; and
a second flexible hinge portion extending from a distal end of the second rigid ring to a proximal end of the third rigid ring.

5. The catheter assembly of claim 4, wherein the second flexible hinge portion fits concentrically between the second rigid ring and the third rigid ring when the telescopic articulation device is in the compressed configuration.

6. The catheter assembly of claim 1, wherein the telescopic articulation device comprises:
a plurality of rigid rings, wherein each of the plurality of rigid rings fits concentrically within a subsequent rigid ring of the plurality of rigid rings; and
a plurality of flexible hinge portions extending between adjacent rigid rings of the plurality of rigid rings.

7. The catheter assembly of claim 6, wherein each flexible hinge portion fits concentrically between the adjacent rigid rings when the telescopic articulation device is in the compressed configuration.

8. The catheter assembly of claim 4, further comprising a proximal connector for attaching to a distal end of the catheter.

9. The catheter assembly of claim 8, wherein the proximal connector is attached to and arranged concentrically within the first rigid ring.

10. The catheter assembly of claim 1 further comprising a handle that is attached to the distal end of the catheter.

11. A delivery system for delivering a medical device to a desired location, the delivery system comprising:
a handle comprising a housing;
a catheter shaft extending from a proximal portion that is attached to the handle to a distal portion, wherein a distal fitting is attached to the distal portion;
a first steering system attached to the handle and for steering the distal portion of the shaft in a first direction and a second direction;
a second steering system attached to the handle and for steering the distal portion of the shaft in a third direction that is different from the first direction and the second direction;
wherein the first steering system is configured such that actuating the first steering system in a first control direction steers the distal portion of the shaft in the first direction and actuating the first steering system in a second control direction steers the distal portion of the shaft in the second direction; and
wherein the second steering system is configured such that actuating the second steering system steers the distal portion of the shaft in the third direction.

12. The delivery system of claim 11, wherein the first steering system comprises a first control member for actuating an actuation element extending from a first portion, which is operatively coupled to the first control member, through the shaft to a second portion, which is coupled to the distal fitting.

13. The delivery system of claim 12, wherein a first grasping element helps operatively couple the first portion of the actuation element to the first control member.

14. The delivery system of claim 11, wherein the first steering system comprises a first control member for rotating a hub and an actuation element extending from a first portion that is attached to the hub, through the shaft, through the distal fitting, back through the shaft, and to a second portion that is attached to the hub.

15. The delivery system of claim 14, wherein a first grasping element attaches a first end of the actuation element to the hub and a second grasping element attaches a second end of the actuation element to the hub.

16. The delivery system of claim 14, wherein the hub comprises a moveable locking portion and the housing comprises a fixed locking portion.

17. The delivery system of claim 16, further comprising a biasing member that biases the hub toward the housing to cause the moveable locking portion to engage the fixed locking portion.

18. The delivery system of claim 16, wherein the first steering system is configured such that actuating the first control member in a non-rotating direction disengages the moveable locking portion from the fixed locking portion.

19. The delivery system of claim 11, wherein the second steering system comprises a second control member for moving an actuation element to move the distal portion of the shaft in the third direction.

20. A delivery system for delivering a medical device to a desired location, the delivery system comprising:
a handle comprising a housing;
a catheter comprising a shaft extending from a proximal end that is attached to the handle to a distal end, wherein a distal fitting is attached to the distal end;
a first steering system attached to the handle and for steering the distal end of the shaft in a first direction and a second direction, the first steering system comprising a first control member for rotating a hub and an actuation element extending from a first portion that is attached to the hub, through the shaft, through the distal fitting, through the shaft, and to a second portion that is attached to the hub;
a second steering system attached to the handle and for steering the distal end of the shaft in a third direction that is different from the first direction and the second direction, the second steering system comprising a second control member for moving an actuation slider to engage the proximal end of the shaft;
wherein rotating the first control member in a first control direction steers the distal end of the shaft in the first direction and rotating the first control member in a second control direction steers the distal end of the shaft in the second direction; and
wherein actuating the second control member steers the distal end of the shaft in the third direction.
